(19) United States
(12) Patent Application Publication
(10) Pub. No.: US 2024/0384250 A1
(43) Pub. Date: Nov. 21, 2024

(54) FIBROBLAST GROWTH FACTOR 23 MODULATING COMPOUNDS

(71) Applicant: THE UAB RESEARCH FOUNDATION, Birmingham, AL (US)

(72) Inventors: Christian FAUL, Vestavia, AL (US); Christopher YANUCIL, Borden Town, NJ (US)

(21) Appl. No.: 18/688,840

(22) PCT Filed: Sep. 6, 2022
(86) PCT No.: PCT/US2022/075989

§ 371 Date: Mar. 4, 2024

Related U.S. Application Data
(60) Provisional application No. 63/240,440, filed on Sep. 3, 2021.

(51) Int. Cl. C12N 9/24; A61K 38/00

(52) U.S. Cl. CPC C12N 9/2402; C12Y 302/01031; A61K 38/00

(57) ABSTRACT

Uses of soluble α-klotho (sKL) to reduce the activity of FGF23 are described. Because FGF23 is implicated in numerous pathologies of humans and other animals, sKL can be used to treat and prevent such pathologies. Other applications of sKL in pharmaceuticals, modulating FGF23 activity, and modulating hypertrophy in cardiac myocytes are described. The disclosure also describes nucleic acids encoding sKL, their complements, vectors for such nucleic acids, and genetically modified organisms containing such nucleic acids.

### CROSS-REFERENCE TO RELATED APPLICATIONS

This application cites the priority of U.S. patent application No. 63/240,440, filed on 3 Sep. 2021 (pending), which is incorporated herein by reference in its entirety.

### STATEMENT REGARDING FEDERAL FUNDING

This invention was made with government support under the National Institutes of Health grant numbers F31DK115074, R01HL128714, and R01HL145528. The government has certain rights in the invention.

In this context “government” refers to the government of the United States of America.

### SUMMARY

The present disclosure describes compounds and methods of their use that provide useful benefits through modulating the activity of fibroblast growth factor 23 (FGF23), although it is to be understood that not all embodiments of the compounds and methods will provide every benefit described. Because FGF23 is associated with numerous conditions and disease states, the compounds and methods disclosed herein can be used to improve such conditions and disease states. It has been discovered that the protein soluble α-klotho (sKL) and some of its derivatives modulate FGF23 in beneficial ways.

A first general embodiment is a method of treating a disease associated with FGF23 in a subject in need thereof comprising: administering a sKL compound to the subject in an amount effective to treat the disease.

A second general embodiment is a method of preventing a disease associated with FGF23 in a subject in need thereof comprising: administering a sKL compound to the subject in an amount effective to prevent the disease.

A third general embodiment is a method of treating a disease associated with FGF23 in a subject in need thereof comprising: increasing the expression of a sKL compound in the subject to an extent effective to treat the disease.

A fourth general embodiment is a method of preventing a disease associated with FGF23 in a subject in need thereof comprising: increasing the expression of a sKL compound in the subject to an extent effective to prevent the disease.

A fifth general embodiment is a medicament for the treatment and/or prevention of a disease associated with FGF23 comprising: a therapeutically effective amount of a sKL compound.

A sixth general embodiment is a sKL compound for use in the treatment or prevention of a disease associated with FGF23.

A seventh general embodiment is a substance for treating or preventing a disease associated with FGF23 comprising a sKL compound as a main ingredient.

An eight general embodiment is a use of a sKL compound for the manufacture of a medicament for the treatment of a disease associated with FGF23.

A ninth general embodiment is a method of modulating the function of FGF23 in a subject comprising administering a sKL compound to the subject.

A tenth general embodiment is a method of reducing or eliminating hypertrophy in a cardiac myocyte associated with FGF23, the method comprising exposing the cardiac myocyte to an effective amount of a sKL compound.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

The present disclosure same can be better understood, by way of example only, with reference to the following drawings. The elements of the drawings are not necessarily to scale relative to each other, emphasis instead being placed upon clearly illustrating the principles of the disclosure.

FIG. 1A shows a gel imaging representation of soluble klotho (sKL) modulating fibroblast growth factor receptor (FGFR) affinities of FGF23 and paracrine FGFs in an FGFR isoform-specific manner. FLAG-tagged sKL or C-terminal bacterial alkaline phosphatase (BAPC) (used as a negative control) was immobilized on anti-FLAG beads and then incubated with recombinant FGF23 protein or solvent (phosphate-buffered saline [PBS]). Protein A/G beads with immobilized anti-FGF23 antibody and incubated with FGF23 served as a positive control for FGF23 precipitation. FLAG-sKL, but not FLAG-BAPC, bound FGF23.

FIG. 1B shows a graphical representation of soluble klotho (sKL) modulating fibroblast growth factor receptor (FGFR) affinities of FGF23 and paracrine FGFs in an FGFR isoform-specific manner. 96-well plates (Thermo Fisher Scientific) were coated with 10 ng of recombinant FGF1, FGF2, FGF7, FGF19, FGF21, or FGF23, washed, and incubated with 40 ng of FLAG-tagged sKL or BAPC. After subsequent washes and incubation with horseradish peroxidase (HRP)-coupled anti-FLAG antibody and HRP substrate, absorbance at 450 nm was measured (presented as arbitrary units [AUs]). Of all the tested FGF isoforms, only FGF23 bound FLAG-sKL. All values are expressed as mean±SEM. n=3 replicate wells. The absorbance at 450 nm surpassed the plate reader limit.

FIG. 1C shows a gel imaging representation of soluble klotho (sKL) modulating fibroblast growth factor receptor (FGFR) affinities of FGF23 and paracrine FGFs in an FGFR isoform-specific manner. Crystallizable fragment (Fc)-tagged FGFR isoforms 1 c, 2c, 3c, and 4 were bound to protein A/G beads and treated with either FLAG-tagged sKL or PBS. FLAG-sKL bound to FGFR4 and to a lower extent to FGFR4.

FIG. 1D shows a graphical representation of soluble klotho (sKL) modulating fibroblast growth factor receptor (FGFR) affinities of FGF23 and paracrine FGFs in an FGFR isoform-specific manner. 96-well plates were coated with 40 ng of FGFR1c or FGFR4 or with 20 ng of FGF23, washed, and incubated with 80 ng of FLAG tagged sKL or BAPC or PBS. Wells were washed again, treated with HRP-coupled anti-FLAG antibody and HRP substrate, and analyzed for absorbance at 450 nm. FLAG-sKL bound FGFR1c and to a lower extent FGFR4 and FGF23. n=3 replicate wells. The absorbance at 450 nm surpassed the plate reader limit.

FIG. 2A shows a gel imaging representation of how sKL and heparin do not modify FGFR affinities of FGF19 and FGF21. FLAG-tagged sKL or BAPC (used as negative control) was immobilized on anti-FLAG beads, and then incubated with recombinant FGF19 protein or solvent (PBS). Protein A/G beads with immobilized anti-FGF19 antibody and incubated with FGF19 served as a positive control for FGF19 precipitations. FLAG-sKL does not binds FGF19.

FIG. 2B shows a gel imaging representation of how sKL and heparin do not modify FGFR affinities of FGF19 and FGF21. FLAG-tagged sKL or BAPC (used as negative control) was immobilized on anti-FLAG beads, and then incubated with recombinant FGF21 protein or solvent (PBS). Protein A/G beads with immobilized anti-FGF21 antibody and incubated with FGF21 served as a positive control for FGF21 precipitations FLAG-sKL does not binds FGF21.

FIG. 2C shows a gel imaging representation of how sKL and heparin do not modify FGFR affinities of FGF19 and FGF21. Fc-tagged FGFR isoforms Ic, 2c, 3c, or 4 were bound to protein A/G beads and treated with either FLAG-tagged sKL or BAPC, or PBS, all in combination with FGF19. Anti-FLAG beads incubated with FLAG-sKL, and anti-FGF19 antibody immobilized on protein A/G beads and treated with FGF19 served as positive controls for FLAG—as well as FGF19-precipitations. Cotreatments with sKL and FGF19 did not lead to complex formation with any of the FGFR isoforms.

FIG. 2D shows a gel imaging representation of how sKL and heparin do not modify FGFR affinities of FGF19 and FGF21. Fc-tagged FGFR isoforms Ic, 2c, 3c, or 4 were bound to protein A/G beads and treated with either FLAG-tagged sKL or BAPC, or PBS, all in combination with FGF21. Anti-FLAG beads incubated with FLAG-sKL, and anti-FGF21 antibody immobilized on protein A/G beads and treated with FGF21 served as positive controls for FLAG—as well as FGF21-precipitations. Cotreatments with sKL and FGF21 did not lead to complex formation with any of the FGFR isoforms.

FIG. 3 shows a graphical representation of how sKL and heparin do not modify FGFR affinities of FGF19 and FGF21. 96-well plates were coated with 4.5 ng of FGF19 or FGF21, washed and then sequentially incubated with 18 ng of FLAG-tagged sKL, 0.4 USP of heparin or vehicle (PBS), 50 ng of Fc-tagged FGFR Ic, 2c, 3c, or 4, HRP-coupled anti-Fc, and HRP substrate, followed by absorbance measurement. The complex of FGF19 and sKL or of FGF21 and sKL did not bind any of the FGFR isoforms. All graphical values are expressed as mean±SEM; for plate based-assays n=3 replicate wells.

FIG. 4A shows a graphical representation of soluble klotho (SKL) modulating fibroblast growth factor receptor (FGFR) affinities of FGF23 and paracrine FGFs in an FGFR isoform-specific manner. 96-well plates were coated with 12.5 ng of FGF2 or FGF7 and incubated with 0.4 United States Pharmacopeia (USP) units of heparin or PBS (vehicle). Wells were washed, incubated with 25 ng of Fc-FGFR1c, washed again, and treated with HRP coupled anti-Fc antibody and HRP substrate, followed by the analysis of absorbance at 450 nm. In reactions receiving sKL, Fc-FGFR1c was preincubated with 50 ng of FLAG-tagged sKL before addition to wells. Heparin increased the binding of FGF2 and FGFS to FGFR1c, but this effect did not occur in the presence of sKL. All values are expressed as mean±SEM. n=3 replicate wells. The absorbance at 450 nm surpassed the plate reader limit.

FIG. 4B shows a graphical representation of soluble klotho (sKL) modulating fibroblast growth factor receptor (FGFR) affinities of FGF23 and paracrine FGFs in an FGFR isoform-specific manner. 96-well plates were coated with 100 ng of FGF5 or FGF8b and incubated with 0.4 United States Pharmacopeia (USP) units of heparin or PBS (vehicle). Wells were washed, incubated with 200 ng of Fc-FGFR1c, washed again, and treated with HRP coupled anti-Fc antibody and HRP substrate, followed by the analysis of absorbance at 450 nm. In reactions receiving sKL, Fc-FGFR1c was preincubated with 400 ng of FLAG-tagged sKL before addition to wells. Heparin increased the binding of FGF2 and FGFS to FGFR1c, but this effect did not occur in the presence of sKL. All values are expressed as mean±SEM. n=3 replicate wells. The absorbance at 450 nm surpassed the plate reader limit.

FIG. 4C shows a gel imaging representation of soluble klotho (sKL) modulating fibroblast growth factor receptor (FGFR) affinities of FGF23 and paracrine FGFs in an FGFR isoform-specific manner. Serum-starved human embryonic kidney 293T (HEK293T) cells were treated with sKL or PBS for 15 minutes, followed by stimulation with FGF2, FGFS, FGF8b, or FGF23 for 10 minutes. Total protein extracts were analyzed using Western blotting. Treatment with FGF2, FGFS, and FGF8b increased levels of phosphorylated extracellular signal-regulated kinase (pERK) in comparison to total levels of ERK (tERK), which did not occur if cells were pretreated with sKL. In contrast, FGF23 treatment increased pERK levels in the presence of sKL. Glyceraldehyde-3-phosphate dehydrogenase (GAPDH) served as a loading control.

FIG. 4D shows a gel imaging representation of soluble klotho (SKL) modulating fibroblast growth factor receptor (FGFR) affinities of FGF23 and paracrine FGFs in an FGFR isoform-specific manner. One microgram of Fc-tagged FGFR isoforms 1c, 2c, 3c, or 4 was bound to protein A/G beads and treated with either 2 μg of FLAG-tagged sKL or BAPC or PBS, all in combination with 500 ng of FGF23. Anti-FLAG beads incubated with either FLAG-SKL or FLAG-BAPC, and anti-FGF23 antibody immobilized on protein A/G beads and treated with FGF23 served as positive controls for FLAG and FGF23 precipitations, respectively. Cotreatments with FGF23 and sKL led to complex formation with FGFR1c and to a lesser extent with FGFR3c and FGFR4.

FIG. 5A shows a graphical representation of soluble klotho (sKL) modulating fibroblast growth factor receptor (FGFR) affinities of FGF23 and paracrine FGFs in an FGFR isoform-specific manner. 96-well plates (Thermo Fisher Scientific) were coated with 4.5 ng of FGF23, washed, and then sequentially incubated with 18 ng of FLAG-tagged sKL or PBS and 50 ng of Fc-tagged FGFR1c, 2c, 3c, or 4, HRP-coupled anti-Fc antibody, and HRP substrate, followed by absorbance measurement. The complex of FGF23 and sKL bound FGFR1 and FGFR4 the strongest, with weaker binding to FGFR3. All values are expressed as mean±SEM. n=3 replicate wells. The absorbance at 450 nm surpassed the plate reader limit.

FIG. 5B shows a graphical representation of soluble klotho (sKL) modulating fibroblast growth factor receptor (FGFR) affinities of FGF23 and paracrine FGFs in an FGFR isoform-specific manner. 96-well plates were coated with 2-fold dilutions of FGF23, ranging from 80 to 2.5 ng. Wells were washed and sequentially incubated with 320 to 10 ng of FLAG-tagged sKL or PBS, followed by 160 to 5 ng of Fc-FGFR1c, 2c, 3c, or 4, HRP-coupled anti-Fc antibody, and HRP substrate. Binding of the SKL/FGFR1c, sKL/FGFR3c, and sKL/FGFR4 complexes occurred in relation to the amount of coated FGF23. In the absence of sKL, FGF23 bound FGFR4 with weak affinity, but not FGFR1c, 2c, or 3c. All values are expressed as mean±SEM. n=3 replicate wells. The absorbance at 450 nm surpassed the plate reader limit.

FIG. 6A shows a graphical representation of production and detection of bioactive soluble klotho (sKL), a fibroblast growth factor (FGF) 23 binding protein with antihypertrophic effects. Neonatal rat ventricular myocytes were cotreated with FGF23 (25 ng/ml) or vehicle (phosphate-buffered saline [PBS]) and with sKL (100 ng/ml), anti-FGF23 blocking antibody (1 μg/ml), or vehicle. FGF23 treatments caused an increase in cell area, while sKL and anti-FGF23 blocked this effect. Comparison between groups was performed using 1-way analysis of variance followed by a post hoc Tukey test. A significance level of P≤0.05 was accepted as statistically significant. n=3 independent isolations, 50 cells per slide, 150 total cells per condition. *P≤0.05 versus all other groups.

FIG. 6B shows a graphical representation of production and detection of bioactive soluble klotho (sKL), a fibroblast growth factor (FGF) 23 binding protein with antihypertrophic effects. Standard curves (linear regression) for the measurements of sKL using the plate-based assay, with FGF23 coated on 96-well plates (Thermo Fisher Scientific) and incubation with Fc-FGFR1c, followed by horseradish peroxidase (HRP)-coupled anti-Fc antibody and HRP for detection. sKL was applied in assay buffer as a diluent. For sKL in buffer, the assay gives a linear range allowing the calculation of sKL concentrations in samples with a limit of detection (LOD) of approximately 300 μg/ml. Absorbances measured are depicted in arbitrary units (AUs) as individual dots and standard curves as straight lines. For plate-based assays, n=3 replicate wells.

FIG. 6C shows a graphical representation of production and detection of bioactive soluble klotho (sKL), a fibroblast growth factor (FGF) 23 binding protein with antihypertrophic effects. Two commonly used commercial preparations of sKL (Com.sKL) and of the first extracellular domain of klotho (KL1) were analyzed in the sKL detection assay in comparison to the sKL. The sKL protein showed approximately 7× higher binding activity than did Com.sKL, and no binding of KL1 was detected. n=3.

FIG. 6D shows a graphical representation of production and detection of bioactive soluble klotho (sKL), a fibroblast growth factor (FGF) 23 binding protein with antihypertrophic effects. Using the FGF23/Fc-FGFR1c-based detection assay, binding affinities of different variants and isoforms of klotho were analyzed, that is, sKL and full-length klotho (KL), as well as soluble β-klotho (sBKL) and full-length β-klotho (BKL) were analyzed. Human embryonic kidney 293T (HEK293T) cells were stably transfected with klotho constructs or a green fluorescent protein (GFP) vector used as a negative control, and total cell lysates were analyzed. A strong signal was observed for sKL and much weaker binding for KL. GFP, sBKL, and BKL showed no measurable binding activity. n=3.

FIG. 7A shows a graphical representation of production and detection of bioactive soluble klotho (SKL), a fibroblast growth factor (FGF) 23 binding protein with antihypertrophic effects. Standard curves (linear regression) for the measurements of sKL using the plate-based assay, with FGF23 coated on 96-well plates (Thermo Fisher Scientific) and incubation with Fc-FGFR1c, followed by horseradish peroxidase (HRP)-coupled anti-Fc antibody and HRP for detection. sKL was applied in rat serum as a diluent. For sKL in serum, the assay is approximately 10× less sensitive, with an LOD of approximately 10 ng/ml. Absorbances measured are depicted in arbitrary units (AUs) as individual dots and standard curves as straight lines. For plate-based assays, n=3 replicate wells.

FIG. 7B shows a graphical representation of production and detection of bioactive soluble klotho (sKL), a fibroblast growth factor (FGF) 23 binding protein with antihypertrophic effects. Rats were injected via the tail vein with sKL (100 μg/kg) or vehicle (PBS), and serial bleeds were taken at different time points and analyzed by the FGF23/Fc-FGFR1c-based detection assay. At the 15-minute time point, high levels of sKL were detected in sKL-injected rats compared with vehicle-injected controls. sKL exhibited a half-life of <15 minutes, and starting at 3 hours postinjection, sKL could no longer be detected. Comparison between groups was performed using 2-tailed t tests. n=3. *P≤0.05 versus vehicle treated at the same time point, ^{#}P≤0.05 versus same treatment in the preceding time point.

FIG. 7C shows a graphical representation of production and detection of bioactive soluble klotho (sKL), a fibroblast growth factor (FGF) 23 binding protein with antihypertrophic effects. Rats were injected with sKL or vehicle described in FIG. 7B, but serial bleeds were taken at earlier time points. After approximately 10 minutes post-injection, sKL levels were reduced by about half. Comparison between groups was performed using 2-tailed t tests. n=3. *P≤0.05 versus vehicle treated at the same time point, ^{#}P≤0.05 versus same treatment in the preceding time point.

FIG. 8A shows a gel image representation of sKL purification and half-life. Analysis of two preparations of recombinant sKL protein from HEK293 cell lysates using a 3-step affinity purification process using a His-tag cobalt column, followed by a Strep-tag column, and eventually a Q column. 30 μL of sample was analyzed by SDS-PAGE and visualized on Coomassie gels. A gradient of BSA standards was used as reference. One band was detected with the expected molecular weight for sKL of 100-150 kDa.

FIG. 8B shows a graphical representation of sKL purification and half-life. Rats were injected via the tail vein with sKL (100 μg/kg) or vehicle (PBS), and serial bleeds were taken at different time points. Phosphate levels were not significantly altered in any of the serum samples. Comparison between groups was performed in form of two-tailed t-tests. A significance level of p≤0.05 was accepted as statistically significant. n=3; *p≤0.05 vs. vehicle treated at same time point, #p≤0.05 vs. same treatment in preceding time point. All values are expressed as mean±SEM.

FIG. 8C shows a graphical representation of sKL purification and half-life. Rats were injected via the tail vein with sKL (100 μg/kg) or vehicle (PBS), and serial bleeds were taken at different time points. Intact FGF23 levels were significantly elevated in some serum samples derived from rats which received sKL, including the 48-hour time point. Comparison between groups was performed in form of two-tailed t-tests. A significance level of p≤0.05 was accepted as statistically significant. n=3; *p≤ 0.05 vs. vehicle treated at same time point, #p≤0.05 vs. same treatment in preceding time point. All values are expressed as mean±SEM.

FIG. 9 shows a schematic representation of the concept of the sKL detection assay. Commercially available ELISA plates (Maxisorp) are coated with 5 μg/ml of recombinant FGF23 protein (R&D Systems) and unspecific binding sites are blocked with BSA/Tween20. Wells are incubated with sKL in solution (which can be plasma, serum or cell culture supernatants) for one hour, washed and incubated for one hour with 750 ng/ml of recombinant FGFR1α(IIIc)-Fc (R&D Systems), which is a chimeric protein consisting of the FGFR1 ectodomain and the Fc region of a human IgG. FGFR1 only binds to FGF23 in the presence of sKL, and the formation of this trimeric sandwich complex is then measured by detecting the presence of the Fc domain that is coupled to FGFR1. To do so, wells are washed and incubated with 400 ng/ml of anti-human IgG conjugated to horseradish peroxidase (HRP; Promega). Wells are washed and incubated with HRP substrate. After development for about 30 minutes, reactions are analyzed on a standard plate reader via absorptions at 450 nM. Incubations with recombinant sKL dissolved in PBS at defined concentrations serve as standards that are used as reference values to determine sKL concentrations in the studied human samples.

FIG. 10A shows a gel image representation of heparin binding fibroblast growth factor receptor isoform 4 (FGFR4), klotho, and FGF23 and increasing the affinity of FGF23 for FGFR4. Crystallizable fragment (Fc)-tagged FGFR1c, 2c, 3c, and 4 were bound to protein A/G beads and treated with FGF23 in combination with either FLAG-tagged soluble klotho (sKL), heparin, or phosphate-buffered saline (PBS). Anti-FLAG beads incubated with FLAG-sKL and anti-FGF23 antibody immobilized on protein A/G beads and treated with FGF23 served as positive controls for FLAG and FGF23 precipitations, respectively. Cotreatments with FGF23 and sKL lead to complex formation with FGFR1c and to a lesser extent with FGFR3c and FGFR4. Cotreatment with heparin increased binding of FGF23 to FGFR4 and to a much lesser extent to the other FGFR isoforms.

FIG. 10B shows a graphical representation of heparin binding fibroblast growth factor receptor isoform 4 (FGFR4), klotho, and FGF23 and increasing the affinity of FGF23 for FGFR4. 96-well plates (Thermo Fisher Scientific) were coated with 250 ng of FGF23, washed, and then sequentially incubated with 0.4 United States Pharmacopeia (USP) units of heparin or vehicle (PBS) and 500 ng of Fc-tagged FGFR1c, 2c, 3c, or 4, horseradish peroxidase (HRP)-coupled anti-Fc, and HRP substrate, followed by absorbance measurement. In the presence of heparin, FGF23 bound FGFR4 the strongest, with weaker binding to FGFR2c and no measurable binding to FGFR1c and FGFR3c.

FIG. 10C shows a graphical representation of heparin binding fibroblast growth factor receptor isoform 4 (FGFR4), klotho, and FGF23 and increasing the affinity of FGF23 for FGFR4. 96-well plates were coated with 2-fold dilutions of FGF23, ranging from 250 to 7.8125 ng. Wells were washed and sequentially incubated with 0.4 USP of heparin or vehicle (PBS), followed by 500 to 15.625 ng of Fc-FGFR1c, 2c, 3c, or 4, HRP-coupled anti-Fc antibody, and HRP substrate. In the presence of heparin, FGF23 did not bind to FGFR1c or 3c, and binding of the heparin/FGFR4 and heparin/FGFR2c complexes occurred in relation to the amount of coated FGF23. In the absence of heparin, FGF23 bound FGFR4 with weak affinity, but not FGFR1c, 2c, or 3c.

FIG. 11A shows a graphical representation of heparin binding fibroblast growth factor receptor isoform 4 (FGFR4), klotho, and FGF23 and increasing the affinity of FGF23 for FGFR4. Surface plasmon resonance (SPR) sensorgrams of the FGFR4-heparin interaction. Concentrations of FGFR4 (from top to bottom): 100, 500, 25, 12.5, and 6.3 nM, respectively. The underlying curves are the fitting curves using model from T200 evaluation software (version 3.2). The biosensor chip response is indicated on the y axis as a function of time (x axis).

FIG. 11B shows a graphical representation of heparin binding fibroblast growth factor receptor isoform 4 (FGFR4), klotho, and FGF23 and increasing the affinity of FGF23 for FGFR4. SPR sensorgrams of the FGF23-heparin interaction. Concentrations of FGF23 (from top to bottom): 100, 500, 25, 12.5, and 6.3 nM, respectively.

FIG. 11C shows a graphical representation of heparin binding fibroblast growth factor receptor isoform 4 (FGFR4), klotho, and FGF23 and increasing the affinity of FGF23 for FGFR4. SPR sensorgrams of the klotho-heparin interaction. Concentrations of sKL (from top to bottom): 500, 250, 125, 63, and 32 nM, respectively.

FIG. 11D shows a graphical representation of heparin binding fibroblast growth factor receptor isoform 4 (FGFR4), klotho, and FGF23 and increasing the affinity of FGF23 for FGFR4. SPR sensorgrams of the FGF23-FGFR4 interaction. Concentrations of FGF23 (from top to bottom): 1000, 500, 250, 125, and 63 nM respectively.

FIG. 11E shows a graphical representation of heparin binding fibroblast growth factor receptor isoform 4 (FGFR4), klotho, and FGF23 and increasing the affinity of FGF23 for FGFR4. Bar graphs based on triplicate experiments with standard deviation, showing normalized FGF23 binding to surface FGFR4 with the addition of different concentrations of heparin in solution.

FIG. 11F shows a graphical representation of heparin binding fibroblast growth factor receptor isoform 4 (FGFR4), klotho, and FGF23 and increasing the affinity of FGF23 for FGFR4. Bar graphs based on triplicate experiments with standard deviation, showing normalized FGF23 binding to surface FGFR4 with the addition of different concentrations of sKL in solution.

FIG. 12A shows a graphical representation of heparin promoting fibroblast growth factor (FGF) 23's prohypertrophic effects in cultured cardiac myocytes. Neonatal rat ventricular myocytes were cotreated with FGF23 at indicated concentrations or vehicle (phosphate-buffered saline PBS) and with heparin (0.19 United States Pharmacopeia units [UP]/ml) or vehicle. FGF23 treatments caused a stepwise elevation in the cell area with increasing FGF23 concentrations. Heparin increased this effect at all FGF23 concentrations. Comparison between groups was performed using 1-way analysis of variance followed by a post hoc Tukey test. n=9. *P≤0.05 versus PBS/PBS, &P≤0.05 versus PBS/heparin; ^{#}P≤0.05 versus FGF23+vehicle at same concentrations. All values are expressed as mean±SEM. For plate-based assays, n=3 replicate wells.

FIG. 12B shows a graphical representation of heparin promoting fibroblast growth factor (FGF) 23's prohypertrophic effects in cultured cardiac myocytes. Adult rat ventricular myocytes (ARVMs) were treated with FGF23 (10 ng/ml), heparin (0.7 USP/ml), or a combination of both, and peak fluorescence [Ca²⁺]; transients (F/F₀) were measured. FGF23 increased [Ca²⁺]; and this change significantly increased in the presence of heparin. Comparison between groups was performed using 1-way analysis of variance followed by a post hoc Tukey test. n=5, n=34 (vehicle); n=3, n=12 (heparin); n=5, n=24 (FGF23); n=4, n=22 (FGF23+heparin). *P≤0.05 versus vehicle, ^{&}P≤0.05 versus heparin. All values are expressed as mean±SEM. For plate-based assays, n=3 replicate wells.

FIG. 13 shows a graphical representation of heparin promoting fibroblast growth factor (FGF) 23's prohypertrophic effects in cultured cardiac myocytes. Representative line scan images of ARVMs under 1 Hz field stimulation perfused with vehicle, heparin, FGF23, or FGF23 combined with heparin for the analysis shown in FIG. 14B.

FIG. 14A shows a graphical representation of heparin promoting fibroblast growth factor (FGF) 23's prohypertrophic effects in cultured cardiac myocytes. ARVMs were treated with FGF23, heparin, or a combination of both, and cell shortening after perfusion was measured. The addition of heparin significantly increased the effect of FGF23. Comparison between groups was performed using 1-way analysis of variance followed by a post hoc Tukey test. n=5, n=34 (vehicle); n=3, n=12 (heparin); n=5, n=24 (FGF23); n=4, n=22 (FGF23+heparin). *P≤0.05 versus vehicle, &P≤0.05 versus heparin. All values are expressed as mean±SEM. For plate-based assays, n=3 replicate wells.

FIG. 14B shows a graphical representation of heparin promoting fibroblast growth factor (FGF) 23's prohypertrophic effects in cultured cardiac myocytes. Intact hearts were isolated from adult mice, attached to a force transducer, paced, and treated with either vehicle, heparin (0.06 USP/ml), FGF23 (9 ng/ml), or a combination of FGF23 and heparin while contractile output was monitored. Waveform changes were analyzed for peak contraction force. FGF23 increased force, which was further elevated in the presence of heparin. Comparison between groups was performed using 1-way analysis of variance followed by a post hoc Tukey test. n=6 (vehicle), n=6 (heparin), n=6 (FGF23), n=8 (FGF23+heparin). *P≤0.05 versus vehicle, &P≤0.05 versus heparin. All values are expressed as mean±SEM. For plate-based assays, n=3 replicate wells.

FIG. 14C shows a graphical representation of heparin promoting fibroblast growth factor (FGF) 23's prohypertrophic effects in cultured cardiac myocytes. FGF23 with heparin also increased the maximum slope of force development of the contractile waveform. Comparison between groups was performed using 1-way analysis of variance followed by a post hoc Tukey test. n=6 (vehicle), n=6 (heparin), n=6 (FGF23), n=8 (FGF23+heparin). *P≤0.05 versus vehicle, &P≤0.05 versus heparin. All values are expressed as mean±SEM. For plate-based assays, n=3 replicate wells.

FIG. 15A shows a graphical representation of how frequent heparin injections aggravate cardiac hypertrophy in mouse models with elevated serum fibroblast growth factor (FGF) 23 levels. Twelve-week-old male BALB/c mice received i.v. injections 2 times per day for 5 consecutive days, with either vehicle (saline), heparin (125 USP/kg), FGF23 (40 μg/kg), or FGF23 (40 μg/kg) plus heparin at 125, 625, or 12.5 USP/kg after which mice underwent echocartliographic analysis and were killed. In mice receiving FGF23 and 125 USP/kg of heparin combined, average wall thickness of the myocardium (T;d) was significantly elevated as compared with vehicle-injected mice. Coinjections of FGF23 with the 2 lower doses of heparin did not result in significant changes. Comparison between groups was performed using 1-way analysis of variance followed by a post hoc Tukey test. A significance level of P 0.05 was accepted as statistically significant. n=4-5. *P≤0.05 versus vehicle, &P≤0.05 versus heparin. All values are expressed as mean±SEM.

FIG. 15B shows a graphical representation of how frequent heparin injections aggravate cardiac hypertrophy in mouse models with elevated serum fibroblast growth factor (FGF) 23 levels. Twelve-week-old male BALB/c mice received i.v. injections 2 times per day for 5 consecutive days, with either vehicle (saline), heparin (125 USP/kg), FGF23 (40 μg/kg), or FGF23 (40 g/kg) plus heparin at 125, 625, or 12.5 USP/kg after which mice underwent echocartliographic analysis and were killed. In mice receiving FGF23 and 125 USP/kg of heparin combined, left ventricular mass in diastole (LV Mass;d) was significantly elevated as compared with vehicle-injected mice. Coinjections of FGF23 with the 2 lower doses of heparin did not result in significant changes. Comparison between groups was performed using 1-way analysis of variance followed by a post hoc Tukey test. A significance level of P 0.05 was accepted as statistically significant. A significance level of P 0.05 was accepted as statistically significant. n=4-5. *P≤0.05 versus vehicle, &P≤0.05 versus heparin. All values are expressed as mean±SEM.

FIG. 15C shows a graphical representation of how frequent heparin injections aggravate cardiac hypertrophy in mouse models with elevated serum fibroblast growth factor (FGF) 23 levels. Twelve-week-old male BALB/c mice received i.v. injections 2 times per day for 5 consecutive days, with either vehicle (saline), heparin (125 USP/kg), FGF23 (40 μg/kg), or FGF23 (40 μg/kg) plus heparin at 125, 625, or 12.5 USP/kg after which mice underwent echocartliographic analysis and were killed. In mice receiving FGF23 and 125 USP/kg of heparin combined, mice receiving FGF23 showed a significant increase in the ratio of heart weight to tibia length (HW/TL) when coinjected with each heparin dose as compared with vehicle-injected mice. Comparison between groups was performed using 1-way analysis of variance followed by a post hoc Tukey test. A significance level of P 0.05 was accepted as statistically significant. A significance level of P 0.05 was accepted as statistically significant. n=4-5. *P≤0.05 versus vehicle, ^{&}P≤0.05 versus heparin. All values are expressed as mean±SEM.

FIG. 16A shows an imaging representation of how frequent heparin injections aggravate cardiac hypertrophy in mouse models with elevated serum fibroblast growth factor (FGF) 23 levels. Twelve-week-old male BALB/c mice received i.v. injections 2 times per day for 5 consecutive days, with either vehicle (saline), heparin (125 USP/kg), FGF23 (40 μg/kg), or FGF23 (40 g/kg) plus heparin at 125, 625, or 12.5 USP/kg after which mice underwent echocartliographic analysis and were killed. Representative hematoxylin and eosin (H&E) staining of cardiac cross-sections. Bar=2 mm.

FIG. 16B shows a graphical representation of how frequent heparin injections aggravate cardiac hypertrophy in mouse models with elevated serum fibroblast growth factor (FGF) 23 levels. Five-week-old male BALB/c mice were administered a 0.2% adenine diet or control chow. After 1 week, mice were i.v. injected 3 times per week with heparin (125 USP/kg) or saline. The ratio of heart weight to body weight (HW/BW) of individual cardiac myocytes were significantly increased in adenine mice injected with heparin. Comparison between groups was performed using 1-way analysis of variance followed by a post hoc Tukey test. A significance level of P 0.05 was accepted as statistically significant. A significance level of P 0.05 was accepted as statistically significant. n=4-5. *P≤0.05 versus vehicle, &P≤0.05 versus heparin, ^{#}P≤0.05 versus adenine. All values are expressed as mean±SEM.

FIG. 17A shows a graphical representation of how frequent heparin injections aggravate cardiac hypertrophy in mouse models with elevated serum fibroblast growth factor (FGF) 23 levels. Five-week-old male BALB/c mice were administered a 0.2% adenine diet or control chow. After 1 week, mice were i.v. injected 3 times per week with heparin (125 USP/kg) or saline. The area of individual cardiac myocytes were significantly increased in adenine mice injected with heparin. Comparison between groups was performed using 1-way analysis of variance followed by a post hoc Tukey test. A significance level of P 0.05 was accepted as statistically significant. n=4-5. *P≤0.05 versus vehicle, ^{&}P≤0.05 versus heparin, ^{#}P≤0.05 versus adenine. All values are expressed as mean±SEM.

FIG. 17B shows an imaging representation of how frequent heparin injections aggravate cardiac hypertrophy in mouse models with elevated serum fibroblast growth factor (FGF) 23 levels. Five-week-old male BALB/c mice were administered a 0.2% adenine diet or control chow. After 1 week, mice were i.v. injected 3 times per week with heparin (125 USP/kg) or saline. Representative H&E staining of cardiac cross-sections from all groups. Bar=2 mm.

FIG. 17C shows an imaging representation of how frequent heparin injections aggravate cardiac hypertrophy in mouse models with elevated serum fibroblast growth factor (FGF) 23 levels. Five-week-old male BALB/c mice were administered a 0.2% adenine diet or control chow. After 1 week, mice were i.v. injected 3 times per week with heparin (125 USP/kg) or saline. Representative images of cardiac wheat germ agglutinin (WGA) staining for the quantification shown in FIG. 17B. Bar=100 μm.

FIG. 18A shows an imaging representation of frequent heparin injections aggravating cardiac hypertrophy in mouse models with elevated serum FGF23 levels. Twelve-week old, male BALB/c mice received i.v. injections, 2× daily, for 5 consecutive days, with either vehicle (saline), heparin (125 USP/kg), FGF23 (40 μg/kg), or FGF23 plus heparin, after which the mice underwent echocardiographic analysis and were sacrificed. Representative B-mode ultrasound images (short axis and long axis view).

FIG. 18B shows a graphical representation of frequent heparin injections aggravating cardiac hypertrophy in mouse models with elevated serum FGF23 levels. Five-week old, female BALB/c were administered a 0.2% adenine diet or control chow. After 1 week, mice were i.v. injected 3× per week with heparin (125 USP/kg) or saline. The ratio of heart weight to body weight was not significantly altered in female mice receiving adenine diet and heparin. Comparison between groups was performed in form of a one-way ANOVA followed by a post-hoc Tukey test. A significance level of p≤0.05 was accepted as statistically significant. n=4-5. *p≤0.05 vs. Vehicle, ^{&}p≤0.05 vs. Heparin, #p≤0.05 vs. Adenine. All values are expressed as mean±SEM.

FIG. 18C shows a graphical representation of frequent heparin injections aggravating cardiac hypertrophy in mouse models with elevated serum FGF23 levels. Five-week old, female BALB/c were administered a 0.2% adenine diet or control chow. After 1 week, mice were i.v. injected 3× per week with heparin (125 USP/kg) or saline. The area of individual cardiac myocytes was not significantly altered in female mice receiving adenine diet and heparin. Comparison between groups was performed in form of a one-way ANOVA followed by a post-hoc Tukey test. A significance level of p≤0.05 was accepted as statistically significant. n=3-5. *p≤0.05 vs. Vehicle, ^{&}p≤0.05 vs. Heparin, #p≤0.05 vs. Adenine. All values are expressed as mean±SEM.

FIG. 19 shows a schematic representation of mechanisms for the FGF co-receptor activities of sKL and heparin. The presence of sKL turns FGFRs into high-affinity FGF23 receptors which signal via the Ras/MAPK cascade and cause cell-type specific effects. In cardiac myocytes, where in absence of klotho FGF23 induces hypertrophy via FGFR4 and PLCy/calcineurin/NFAT signaling, sKL protects from FGF23 effects by switching FGF23-induced downstream signaling to the Ras/MAPK pathway and possibly by switching to FGFR1c as the FGF23 receptor. In contrast, sKL blocks binding of certain paracrine FGFs, such as FGF2, to FGFR1c and thereby Ras/MAPK signaling. Inhibition of the prohypertrophic actions of FGF23 and the mitogenic effects of paracrine FGF might underlie the pleiotropic tissue-protective effects of sKL. Heparin is an established co-factor for the binding of paracrine FGFs to FGFR1c and the induction of Ras/MAPK signaling. Heparin also increases the binding of FGF23 specifically to FGFR4, which in the absence of klotho, as the case in cardiac myocytes, results in increased PLCy/calcineurin/NFAT signaling and aggravated cardiac injury.

FIG. 20 shows a schematic representation of a Strep/His-tagged sKL.

FIG. 21A shows a gel image representation of a purified sKL protein and the concept of the sKL detection assay. Analysis of two preparations of recombinant sKL protein from HEK293 cell lysates using a Histag cobalt column, followed by a Strep-tag column and a Q column. 30 ul of sample was analyzed by SDS-PAGE and visualized on Coomassie gels.

FIG. 21B shows a schematic representation of a purified sKL protein and the concept of the sKL detection assay. 96-well plates were coated with 5 μg/ml of recombinant FGF23 protein and unspecific binding sites were blocked with BSA/Tween20. Wells were incubated with sKL (which can be plasma, serum or cell culture supernatants) for one hour, washed and incubated for one hour with 750 ng/ml of a chimeric protein consisting of the FGFR1c ectodomain and the Fc region of a human IgG. FGFR1c only binds to FGF23 in the presence of sKL, and the formation of this trimeric sandwich complex was measured by detecting the presence of the Fc domain in FGFR1c. Wells were washed and incubated with 400 ng/ml of anti-human IgG conjugated to horseradish peroxidase (HRP; Promega). Wells were washed and incubated with HRP substrate. After development for about 30 minutes, reactions were analyzed on a standard plate reader via absorptions at 450 nM. Incubations with recombinant sKL dissolved in PBS at defined concentrations served as standards used as reference values to determine sKL concentrations in the studied samples.

FIG. 22A shows a graphical representation of the detection of bioactive sKL protein. Standard curves for the measurements of FLAG-sKL using the plate-based assay, with FGF23 coated on well and incubations with Fc-FGFR1c. sKL was applied in assay buffer. For sKL in buffer, the assay gives a linear range with a limit of detection (LOD) of around 300 μg/mL. Absorbances measured are depicted in arbitrary units (A.U.).

FIG. 22B shows a graphical representation of the detection of bioactive sKL protein. Two commercial preparations of sKL (Com.sKL from R&D Systems) and KL1 (from PeproTech) were analyzed in comparison to the FLAG-sKL protein. n=3.

FIG. 22C shows a graphical representation of the detection of bioactive sKL protein. Binding affinities of different variants and isoforms of klotho, i.e. sKL and full-length klotho (KL) were analyzed, as well as soluble β-klotho (sBKL) and full-length β-klotho BKL). HEK293T cells were stably transfected with FLAG-klotho constructs or a GFP vector used as negative control, and total cell lysates were analyzed. n=3.

FIG. 22D shows a graphical representation of the detection of bioactive sKL protein. Standard curves for the measurements of FLAG-sKL using the plate-based assay, with FGF23 coated on well and incubations with Fc-FGFR1c. sKL was applied in rat serum. For sKL in serum, the assay is about 10× less sensitive, with an LOD of around 10 ng/ml. Absorbances measured are depicted in arbitrary units (A.U.).

FIG. 22E shows a graphical representation of the detection of bioactive sKL protein. Rats were injected via the tail vein with FLAG-sKL (100 g/kg) or vehicle (PBS), and serial bleeds were taken at different time points and analyzed by a FGF23/FGFR1c-based detection assay. Comparison between groups was performed in form of a two-tailed t-tests. n=3; *p≤0.05 vs. vehicle treated at same time point, #p≤0.05 vs. same treatment in preceding time point; for plate-based assays n=3 replicate wells.

FIG. 23A shows a graphical representation of sKL binding FGFR1c and FGFR4 and protecting cultured cardiac myocytes from FGF23-induced hypertrophy. 96-well plates were coated with 4.5 ng of FGF23, washed and then sequentially incubated with 18 ng of FLAG-sKL or PBS, 50 ng of Fc-FGFR 1c, 2c, 3c, or 4, HRP coupled anti-Fc, and HRP substrate. All graphical values are expressed as mean±SEM; for plate-based assays n=3 replicate wells.

FIG. 23B shows a graphical representation of sKL binding FGFR1c and FGFR4 and protecting cultured cardiac myocytes from FGF23-induced hypertrophy. Plates were coated with 40 ng of FGFR1c or FGFR4, or with 20 ng of FGF23, washed and incubated with 80 ng of FLAG-tagged sKL or BAPC (negative control), or PBS. All graphical values are expressed as mean±SEM; for plate-based assays n=3 replicate wells.

FIG. 23C shows a graphical representation of sKL binding FGFR1c and FGFR4 and protecting cultured cardiac myocytes from FGF23-induced hypertrophy. NRVMs were co-treated with FGF23 (25 ng/mL) or vehicle (PBS), and with FLAG-sKL (100 ng/ml), an anti-FGF23 blocking antibody (1 μg/mL; Amgen) or vehicle. After 48 hours cells were fixed and immunostained, and the cell area was determined by fluorescence microscopy. n=3 independent isolations, 50 cells per slide, 150 total cells per condition; *p≤0.05 vs. all other groups; Comparison between groups was performed in form of a one-way ANOVA followed by a post-hoc Tukey test.

FIG. 24A shows a graphical representation of sKL inhibiting the binding of paracrine FGFs to FGFR1c and subsequent signaling. 96-well plates were coated with 12.5 ng of FGF2 or FGF7 and incubated with 0.4 USP heparin or PBS (vehicle). Wells were washed, incubated with 25 ng of Fc-FGFR1c, washed again, treated with HRP coupled anti-Fc, and HRP substrate. In reactions receiving SKL, Fc-FGFR1c was pre-incubated with 50 ng of FLAG-sKL prior to the addition to wells. All graphical values are expressed as mean±SEM; for plate-based assays n=3 replicate wells.

FIG. 24B shows a graphical representation of sKL inhibiting the binding of paracrine FGFs to FGFR1c and subsequent signaling. 96-well plates were coated with 100 ng of FGF5 or FGF8b and incubated with 0.4 USP heparin or PBS (vehicle). Wells were washed, incubated with 200 ng of Fc-FGFR1c, washed again, treated with HRP coupled anti-Fc, and HRP substrate. In reactions receiving SKL, Fc-FGFR1c was pre-incubated with 400 ng of FLAG-sKL prior to the addition to wells. All graphical values are expressed as mean±SEM; for plate-based assays n=3 replicate wells.

FIG. 24C shows a gel image representation of sKL inhibiting the binding of paracrine FGFs to FGFR1c and subsequent signaling. Serum-starved HEK293T cells were treated with FLAG-sKL or PBS for 15 minutes, followed by stimulation with FGF2, FGF5, FGF8b, or FGF23 for 10 minutes. Protein extracts were analyzed by Western blotting for levels of phosphorylated (PERK) and total ERK (tERK), GAPDH served as loading control.

FIG. 25A shows a graphical representation and gel image of reducing sKL glycosylation to increase FGF23 binding affinity. 96-well plates were coated with 100 ng of FGF23 or 200 ng of FGFR1c and incubated with sKL-FLAG from HEK293 cell lysate or from media. Values were normalized to the sKL amounts in lysate versus media as determined by anti-FLAG Western blotting. Wells were washed and incubated with anti-FLAG HRP.

FIG. 25B shows a graphical representation and gel image of reducing sKL glycosylation to increase FGF23 binding affinity. Plates were coated with 100 ng of FGF23 or 100 ng of FGFR1c and incubated with media from sKL-expressing HEK293 that were treated with 2 mM glucosamine (Glucos) or 12.5 ng/ml tunicamycin (Tunic) for 48 hours. All graphical values are expressed as mean±SEM; for plate-based assays n=3 replicate wells.

FIG. 25C shows a gel image representation of reducing sKL glycosylation to increase FGF23 binding affinity. HEK293 cells were transfected with FLAG-sKL variants with mutated glycosylation sites (Asn (N) to Glu (Q) substitutions). Cells were serum starved for 48 hours, and sKL-FLAG in media was analyzed by immunoblotting.

FIG. 25D shows a graphical representation of reducing sKL glycosylation to increase FGF23 binding affinity. Plates were coated with 100 ng of FGF23 or 200 ng of FGFR1c and incubated with media containing FLAGsKL variants from C.

FIG. 25E shows a graphical representation of reducing sKL glycosylation to increase FGF23 binding affinity. Plates were coated with 100 ng of FGF23 or 200 ng of FGFR1c and incubated with 400 ng of purified FLAG-tagged wildtype sKL or sKL-N694Q.

FIG. 25F shows a graphical representation of reducing sKL glycosylation to increase FGF23 binding affinity. Plates were coated with 100 ng of FGF23 and incubated with 400 ng of purified FLAG-tagged wildtype sKL or sKL-N694Q. Wells were washed and incubated with 200 ng of Fc-FGFR1c.

### DETAILED DESCRIPTION

### Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated.

With reference to the use of the word(s) “comprise,” “comprises,” and “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “including” should be interpreted to mean “including but not limited to . . . ” unless the context clearly indicate otherwise.

The term “consisting essentially of” means that, in addition to the recited elements, what is claimed may also contain other elements (steps, structures, ingredients, components, etc.) that do not adversely affect the operability of what is claimed for its intended purpose. Such addition of other elements that do not adversely affect the operability of what is claimed for its intended purpose would not constitute a material change in the basic and novel characteristics of what is claimed.

The term “adapted to” means designed or configured to accomplish the specified objective, not simply able to be made to accomplish the specified objective.

The term “capable of” means able to be made to accomplish the specified objective.

The terminology used herein is for the purpose of describing particular embodiments only and is not intended to be limiting. As used herein, the singular forms “a”, “an” and “the” are intended to include the plural forms as well (i.e. “at least one”), unless the context clearly indicates otherwise.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The terms “prevention”, “prevent”, “preventing”, “suppression”, “suppress” and “suppressing” as used herein refer to a course of action initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or device of the present disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method or device of the disclosure.

In this disclosure terms such as “administering” or “administration” include acts such as prescribing, dispensing, giving, or taking a substance such that what is prescribed, dispensed, given, or taken is actually contacts the patient's body externally or internally (or both). It is specifically contemplated that instructions or a prescription by a medical professional to a subject or patient to take or otherwise self-administer a substance is an act of administration. Administration also includes prescribing, giving, implanting, or otherwise positioning on or in a subject a medical device that functions to dispense or give the substance.

Terms such as “at least one of A and B” should be understood to mean “only A, only B, or both A and B.” The same construction should be applied to longer list (e.g., “at least one of A, B, and C”).

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

The term “prodrug” as used herein includes functional derivatives of a disclosed compound which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985.

### Active Compounds

Compounds are provided that reduce the activity of FGF23. Without wishing to be bound by any hypothetical model, it is believed that the active compounds bind to FGF23 and inhibit binding between FGF23 and FGF receptors that would otherwise result in numerous conditions and disease states. Compounds that produce this effect are referred to as “active compounds” herein.

The active compounds are sKL compounds. The term “sKL compounds” refers to compounds comprising a polypeptide sequence having a level of sequence identity with a fragment of sKL from a chordate, and derivatives thereof. Examples of chordates in which KL sequences are known or predicted include Homo sapiens (NCBI Reference Sequence: NP_004786.2, SEQ ID NO: 1), Pan troglodytes (NCBI Reference Sequence: XP_522655.2, SEQ ID NO: 2), Macaca mulatta (NCBI Reference Sequence: XP_001101127.1, SEQ ID NO: 3), Canis lupus familiaris (NCBI Reference Sequence: XP_003433357.1, SEQ ID NO: 4), Bos Taurus (NCBI Reference Sequence: NP_001178124.1, SEQ ID NO: 5), Mus musculus (NCBI Reference Sequence: NP_038851.2, SEQ ID NO: 6), Rattus norvegicus (NCBI Reference Sequence: NP_112626.1, SEQ ID NO: 7), Gallus gallus (NCBI Reference Sequence: XP_004938814.1, SEQ ID NO: 8), Danio rerio (NCBI Reference Sequence: XP_690797.4, SEQ ID NO: 9), and Xenopus tropicalis (NCBI Reference Sequence: XP_002934067.1, SEQ ID NO: 10). The forgoing reference sequences are incorporated by reference into this document.

Klotho is a transmembrane protein found on Chromosome 13 in H. sapiens. It has multiple known isoforms produced by alternative splicing. The canonical human isoform is 1012 amino acids long and 116, 181 Da in mass. Klotho was previously believed to promote the binding of FGF proteins (including FGF23) to their corresponding receptors (Kuro-o, Nature Rev. Nephrology, 15:27-44 (2019)).

Soluble klotho (sKL) is produced by the cleavage of the KL ectodomain. There is some debate as to exactly where cleavage occurs to produce sKL. In this disclosure it will be assumed that sKL corresponds to positions 34-981 of H. sapiens KL or positions 35-982 of Mus musculus KL. The sKL compounds of this disclosure comprise a polypeptide having at least 70% sequence identity with a fragment of positions 34-981 of H. sapiens KL (SEQ ID NO: 1), positions 35-982 of Mus musculus KL (SEQ ID NO: 2), or a corresponding region of KL of another species. Such corresponding regions can be determined by constructing an alignment between mouse or human KL with the KL of the other species. An example of such an alignment is shown in Table 5. When amino acid positions of sKL are referenced in this disclosure, these positions are based on the sequence of KL; for example, position 34 of human sKL actually refers to the first amino acid in human sKL are currently understood. Again, because the exact cleavage location when sKL is cleaved from KL is a matter of debate, the better established sequence of KL is used for reference.

The present disclosure contemplates the use of sKL derivatives in the methods and compositions disclosed herein. A sKL derivative as defined herein refers to a sKL polypeptide that includes a one or more fragments, insertions, deletions or substitutions. The sKL derivative may have an activity that is comparable to or increased (in one embodiment, 50% or more) as compared to the wild-type sKL activity and as such may be used to increase a sKL activity; alternatively, the sKL derivative may have an activity that is decreased (in one embodiment, less than 50%) as compared to the wild-type sKL activity and as such may be used to decrease a sKL activity. In some cases the derivative will retain antigenic specificity of sKL.

A fragment of sKL is any polypeptide consisting of any number of adjacent amino acid residues having the same identity and order as any segment of sKL. Conservative modifications to the amino acid sequence of any fragment are also included (conservative substitutions are discussed below). Such fragments can be produced for example by digestion of sKL with an endoprotease (which will produce two or more fragments) or an exoprotease. A fragment may be of any length up to the length of sKL. A fragment may be, for example, at least 3 residues in length. A fragment that is at least 6 residues in length will generally function as an antigenic group. Such groups would be expected to be cross-recognized by some antibodies specific for sKL. Fragments that are homologous to parts of the KL2 domain and/or the linker region of sKL may have FGF23 modulating activity. Without wishing to be bound to any hypothetical model, it is believed that klotho consists of a short cytoplasmic tail of unknown function. The extracellular part of klotho, which is the major part of klotho, and also exists in a soluble from, i.e, soluble klotho, which is separated in a KL1 and KL2 domain, and a linker region connecting the two. It is believed that KL2 binds FGFRs and KL2 and the linker region bind FGF23. It is believed that KL1 is not involved in FGFR or FGF23 binding, and its role is not clear. KL1 could have effects on proper folding/extension of the klotho protein. However, it is possible that the KL2 and linker regions have the same function/activity as whole sKL, as the current understanding of sKL function is still developing. For an understanding of the functions and structures of the KL1, KL2, and linker regions of sKL, the reader is referred to “α-Klotho is a non-enzymatic molecular scaffold for FGF23 hormone signalling.” Chen G, et al. (2018) Nature January 25; 553 (7689): 461-466; and “The Klotho proteins in health and disease.” Kuro-O M. Nat Rev Nephrol. (2019) January; 15 (1): 27-44

Derivatives of sKL will have some degree of sequence identity with native sKL. It would be expected that most derivatives having from 95-100% identity with native sKL would retain the function of sKL; likewise derivatives with higher levels of identify would as well, e.g. at least 96%, 97%, 98%, 99%, and 99.5%. It would also be predicted that the likelihood that functionality would be retained by a homolog to sKL within any one of the following ranges of identity: 75-100%, 80-100%, 85-100%, and 90-100%. The minimum desirable identity can be determined in some cases by identifying a known non-functional homolog to sKL, and establishing that the minimum desirable identity must be above the identity between sKL and the known non-functional identity. The minimum desirable identity can also be determined in some cases by identifying a known functional homolog to sKL, and establishing that the range of desirable identity must encompass the percent identity between sKL and the known non-functional identity.

The deletions, additions and substitutions can be selected to generate a desired sKL derivative. For example, while not wishing to be bound by any hypothetical model, based on the current understanding it is not expected that deletions, additions and substitutions in the KL1 region of a sKL would alter a sKL activity. However, it is possible that the KL1 region has other desirable functions, and in some embodiments of the sKL compound the KL1 region is conserved or subject only to conservative substitutions. Likewise conservative substitutions or substitutions of amino acids with similar properties is expected to be tolerated in the KL2 and linker regions, and a sKL activity may be conserved. Of course non-conservative substitutions in these regions would be expected to decrease or eliminate a sKL activity. In addition, specific deletions, insertions and substitutions may impact, positively or negatively, a certain sKL activity but not impact another sKL activity.

Conservative modifications to the amino acid sequence of any of SEQ ID NOS: 1-10, including combinations thereof (and the corresponding modifications to the encoding nucleotides) will be expected to produce sKL derivatives having functional and chemical characteristics similar to those of naturally occurring sKL. In contrast, substantial modifications in the functional and/or chemical characteristics of sKL may be accomplished by selecting substitutions in the amino acid sequence of any of SEQ ID NOS: 1-10, including combinations thereof, that differ significantly in their effect on maintaining (a) the structure of the molecular backbone in the area of the substitution, (b) the charge or hydrophobicity of the molecule at the binding site for a binding target, or (c) the bulk of a side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine.

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. It will be appreciated by those of skill in the art that nucleic acid and polypeptide molecules described herein may be chemically synthesized as well as produced by recombinant means.

Naturally occurring residues may be divided into classes based on common side chain properties. Hydrophobic residues are norleucine, Met, Ala, Val, Leu, 11e. Neutral hydrophilic residues are: Cys, Ser, Thr, Asn, Gln. Acidic residues are: Asp, Glu. Basic residues are: His, Lys, Arg. Residues that influence chain orientation are: Gly, Pro. Aromatic residues are: Trp, Tyr, Phe.

For example, non-conservative substitutions may involve the exchange of a member of one of these classes for a member from another class.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity.

In making changes based upon the hydropathic index, the substitution of amino acids whose hydropathic indices are within +/−2 may be used; in an alternate embodiment, the hydropathic indices are with +/−1; in yet another alternate embodiment, the hydropathic indices are within +/−0.5.

It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 may be used; in an alternate embodiment, the hydrophilicity values are with ±1; in yet another alternate embodiment, the hydrophilicity values are within ±0.5.

Desired amino acid substitutions (whether conservative or non-conservative) can be determined by those skilled in the art at the time such substitutions are desired. For example, amino acid substitutions can be used to identify important residues of the sKL, or to increase or decrease the affinity of the sKL with a particular binding target in order to increase or decrease a sKL activity. Exemplary amino acid substitutions are set forth in Table 1.

TABLE 1

| Original | | |
|---|---|---|
| Amino | Exemplary | Preferred |
| Acid | substitution | substitution |
| Ala | Val, Leu, Ile | Val |
| Arg | Lys, Gln, Asn | Lys |
| Asn | Glu | Glu |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Ile, Val, Met, Ala, Phe, Norleucine | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Suitable variants of the polypeptide as set forth in any of SEQ ID NOS: 1-10 can be determined, including combinations thereof, using well known techniques. For identifying suitable areas of the molecule that may be changed without destroying activity, areas may be targeted that are not believed to be important for activity. For example, when similar polypeptides with similar activities from the same species or from other species are known, the amino acid sequence of a sKL may be compared to such similar polypeptides. With such a comparison, one can identify residues and portions of the molecules that are conserved among similar polypeptides. It will be appreciated that changes in areas of a sKL that are not conserved relative to such similar polypeptides would be less likely to adversely affect the biological activity and/or structure of the sKL. One may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one can review structure-function studies identifying residues in similar polypeptides that are important for activity or structure. In view of such a comparison, one can predict the importance of amino acid residues in a sKL that correspond to amino acid residues that are important for activity or structure in similar polypeptides. One may opt for chemically similar amino acid substitutions for such predicted important amino acid residues of sKL.

One skilled in the art can also analyze the three-dimensional structure and amino acid sequence in relation to that structure in similar polypeptides. In view of that information, one skilled in the art may predict the alignment of amino acid residues of a sKL with respect to its three dimensional structure. One skilled in the art may choose not to make radical changes to amino acid residues predicted to be on the surface of the protein, since such residues may be involved in important interactions with other molecules. Moreover, one skilled in the art may generate test sKL derivatives containing a single amino acid substitution at each desired amino acid residue. The derivatives can then be screened using activity assays known to those skilled in the art and as disclosed herein. Such derivatives could be used to gather information about suitable substitution. For example, if one discovered that a change to a particular amino acid residue resulted in destroyed, undesirably reduced, or unsuitable activity, derivatives with such a change would be avoided. In other words, based on information gathered from such routine experiments, one skilled in the art can readily determine the amino acids where further substitutions should be avoided either alone or in combination with other mutations.

Numerous scientific publications have been devoted to the prediction of secondary structure from analyses of amino acid sequences (see Chou et al., Biochemistry, 13 (2): 222-245, 1974; Chou et al., Biochemistry, 113 (2): 211-222, 1974; Chou et al., Adv. Enzymol. Relat. Areas Mol. Biol., 47:45-148, 1978; Chou et al., Ann. Rev. Biochem., 47:251-276, 1979; and Chou et al., Biophys. J., 26:367-384, 1979). Moreover, computer programs are currently available to assist with predicting secondary structure of polypeptides. Examples include those programs based upon the Jameson-Wolf analysis (Jameson et al., Comput. Appl. Biosci., 4 (1): 181-186, 1998; and Wolf et al., Comput. Appl. Biosci., 4 (1): 187-191; 1988), the program PepPlot® (Brutlag et al., CABS, 6:237-245, 1990; and Weinberger et al., Science, 228:740-742, 1985), and other new programs for protein tertiary structure prediction (Fetrow. et al., Biotechnology, 11:479-483, 1993).

Moreover, computer programs are currently available to assist with predicting secondary structure. One method of predicting secondary structure is based upon identity modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure (see Holm et al., Nucl. Acid. Res., 27 (1): 244-247, 1999).

Additional methods of predicting secondary structure include “threading” (Jones, D., Curr. Opin. Struct. Biol., 7 (3): 377-87, 1997; Suppl et al., Structure, 4 (1): 15-9, 1996), “profile analysis” (Bowie et al., Science, 253:164-170, 1991; Gribskov et al., Meth. Enzym., 183:146-159, 1990; and Gribskov et al., Proc. Nat. Acad. Sci., 84 (13): 4355-4358, 1987), and. “evolutionary linkage” (See Home, supra, and Brenner, supra).

Some embodiments of the sKL compound will have reduced glycosylation compared to a wild type sKL. The reduced glycosylation may be produced in various ways (“hypo-glycosylated”). The hypo-glycosylated sKL compound may be produced by removing one or more glycosyl groups from a more highly glocosylated or fully glycosylated sKL compound. The hypo-glycosylated sKL compound may also be produced by synthesizing the polypeptide in an un-glycosylated state or a hypo-glycosylated state. The hypo-glycosylated sKL compound may be produced by substituting the amino acids at one or more glycosylation sites with substituted amino acids that are not subject to glycosylation. For example, canonical human sKL is believed to have glycosylation asparagine sites at positions 106, 159, 283, 344, 607, 612, 630, and 694. As another example, canonical mouse sKL is believed to have glycosylation asparagine sites at positions 161, 285, 346, 609, 614, and 696. Some embodiments of the hypo-glycosylated sKL compound have substitutions at one or more of the foregoing asparagines in human or mouse sKL. In further embodiments the one or more substitutions is an N->Q substitution. Other embodiments the hypo-glycosylated sKL compound may have one or more point deletions at one or more of the foregoing asparagines in human or mouse sKL. In embodiments in which the sKL compound is derived from another species of another isoform of sKL, substitutions may be made at corresponding positions on the polypeptide. More specific embodiments of the sKL compound comprise a substitution at one or more of positions 106, 159, 283, 344, 607, 612, 630, and 694 of human sKL, or the corresponding positions in sKL of another species. A more preferred embodiment comprises a substitution at position 694 of human sKL or the corresponding position in sKL of another species. A further preferred embodiment comprises a N->Q substitution at position 694. A still further preferred embodiment of the sKL compound is isogenic with wild-type, except for one substitution at position 694.

The sKL compound may have one or more substitutions at any position. Embodiments of the sKL based on human sKL may have one or more substitutions at one or more of positions 34-981 (using KL as a reference). Embodiments of the sKL based on mouse sKL may have one or more substitutions at one or more of positions 35-982. Embodiments of the sKL compound based on other species sKL may have one or more substitutions at one or more of positions corresponding to positions 34-981 in human sKL. Specific embodiments of the sKL compound may have substitutions at one or more positions corresponding to the following substitutions in human or mouse sKL: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, and 982.

The active compounds may be pharmaceutically acceptable salts, esters, amides, and prodrugs of the sKL compound. The pharmaceutically acceptable salt may be a salt of the active compounds which are prepared with relatively nontoxic acids or bases, depending on the particular substituents found on the compounds described herein. If the sKL compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When the sKL compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). If the sKL compounds contain both basic and acidic functionalities, the compounds may be converted into either base or acid addition salts.

### Methods of Treatment and Prevention

In one general embodiment, the teachings of the present disclosure provide for the treatment and/or prevention of disease states and conditions associated with FGF23 in a subject in need of such treatment. Such disease states and conditions include, but are not limited to: kidney disease, chronic kidney disease, hyperphosphatemia, cardiovascular disease, vascular calcification, cardiovascular fibrosis, stress-induced cardiac injury, apoptosis, oxidative stress, cellular senescence, normal aging, tissue fibrosis, and inflammation. The method comprises administering an effective amount of the active agent to treat or prevent the disease state or condition. An sKL compound is also provided for use in the treatment and/or prevention of a disease associated with fibroblast growth factor FGF23. A substance for treating or preventing a disease associated with fibroblast FGF23, is also provided comprising an sKL compound as a main ingredient.

Some embodiments of the methods and uses comprise administering 10-1000 μg active agent per kg of the intended subject (hereinafter, simply “μg/kg”). Further embodiments of the method comprise administering the active agent at about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg of the intended subject. Some embodiments of the method comprise administering an amount of active agent that is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg of the intended subject. Some embodiments of the method comprise administering an amount of active agent that is at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg of the intended subject. Various embodiments of the composition may contain a range of active agent between any one of the minima disclosed above and any one of the maxima disclosed above, with the caveat that the minimum must be lower than the maximum in the range. In a specific embodiment the active agent is administered at 100 μg/kg of the intended subject. In some embodiments of the method multiple active agents disclosed herein may be administered (e.g, different sKL compounds). In such embodiments each of the active agents may be administered in an independently selected therapeutically effective amount.

The method of treatment and/or prevention comprises administering to the subject any of the active compounds disclosed herein. The method will often further comprise identifying a subject in need of such treatment or prevention. In one embodiment, said inhibition or modulation is accomplished by decreasing activity, in whole or in part, of FGF23. Such decreased activity is accomplished by administering an active compound described above.

Another approach to treatment and/or prevention of the FGF23-associated disease is increasing the expression of a soluble α-klotho compound in the subject to an extent effective to treat the disease. Such increase in expression may be accomplished by any suitable approach. For example, the subject may be genetically modified to increase expression through the introduction of a nucleic acid encoding a more active sKL compound, additional copies of a nucleic acid encoding a an sKL compound, a more active promoter operatively linked to a sequence encoding an sKL compound, and/or an enhancer operatively linked to a sequence encoding an sKL compound. In further embodiments of the method expression may be increased through use of an activator that targets either a promotor or an enhancer.

A method of preventing a disease associated with FGF23 in a subject in need thereof comprising: increasing the expression of a soluble α-klotho compound in the subject to an extent effective to prevent the disease.

### Pharmaceutical Compositions

Pharmaceutical compositions are provided comprising any of the active agents described above, as are uses of the active agents for the manufacture of a medicament for treatment of a disease associated with FGF23. The pharmaceutical compositions may comprise one or more of such active agents, in combination with a pharmaceutically acceptable carrier. Examples of such carriers and methods of formulation may be found in Remington: The Science and Practice of Pharmacy (20^{th} Ed., Lippincott, Williams & Wilkins, Daniel Limmer, editor). To form a pharmaceutically acceptable composition suitable for administration, such pharmaceutical compositions will contain a therapeutically effective amount of a compound(s).

The pharmaceutical compositions of the disclosure may be used in the treatment and prevention methods of the present disclosure. Such pharmaceutical compositions are administered to a subject in amounts sufficient to deliver a therapeutically effective amount of the active agent so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any suitable method. Exemplary routes of administration include, but are not limited to, subcutaneous, intravenous, topical, epicutaneous, oral, intraosseous, intramuscular, intranasal and pulmonary. The pharmaceutical compositions of the present disclosure may be administered only one time to the subject or more than one time to the subject. Furthermore, when the pharmaceutical compositions are administered to the subject more than once, a variety of regimens may be used, such as, but not limited to, one per day, once per week, once per month or once per year. The pharmaceutical compositions may also be administered to the subject more than one time per day. The therapeutically effective amount of the pharmaceutical composition and appropriate dosing regimens may be identified by routine testing in order to obtain optimal activity, while minimizing any potential side effects. In addition, co-administration or sequential administration of other agents may be desirable. Some embodiments of the composition contain a mass of the active agent that is 1-1000 μg/kg of the intended subject; further embodiments of the composition contain a mass of the active agent that is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg of the intended subject. Some embodiments of the composition contain a mass of the active agent that is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg of the intended subject. Some embodiments of the composition contain a mass of the active agent that is at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg of the intended subject. Various embodiments of the composition may contain a range of active agent between any one of the minima disclosed above and any one of the maxima disclosed above, with the caveat that the minimum must be lower than the maximum in the range.

The pharmaceutical compositions of the present disclosure may be administered systemically, such as by intravenous administration, or locally such as by subcutaneous injection or by application of a paste or cream.

The pharmaceutical compositions of the present disclosure may further comprise agents which improve the solubility, half-life, absorption, etc. of the compound(s). Furthermore, the pharmaceutical compositions of the present disclosure may further comprise agents that attenuate undesirable side effects and/or or decrease the toxicity of the compounds(s). Examples of such agents are described in a variety of texts, such a, but not limited to, Remington: The Science and Practice of Pharmacy (20th Ed., Lippincott, Williams & Wilkins, Daniel Limmer, editor).

The pharmaceutical compositions of the present disclosure can be administered in a wide variety of dosage forms for administration. For example, the pharmaceutical compositions can be administered in forms, such as, but not limited to, tablets, capsules, sachets, lozenges, troches, pills, powders, granules, elixirs, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

The pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the nucleic acid molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable and coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the nucleic acid molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The compound(s) of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The compound(s) of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### Methods of Modulating FGF23 and Cardiac Cells

The sKL compound has uses in addition to methods of treatment and prevention described above.

The sKL compound can be used to modulate the function of FGF23. A method of modulating FGF23 function is provided comprising contacting FGF23 with any of the sKL compounds disclosed above. In some embodiments of the method, the FGF23 is found in an organism, such as an animal subject. In such embodiments the method comprises administering the sKL compound to the subject in an amount effective to modulate FGF23. The modulation may be a decrease in the binding of FGF23 to an FGFR. In some embodiments of the method the FGFR is FGFR1c, FGFR2c, FGFR3c, FGFR4, any two of the foregoing, any three of the foregoing, or all of the foregoing. The concentration of the sKL compound will be sufficient to accomplish said modulation, such as a decrease in the binding of FGF23 to the FGFR. In some embodiments of the method the concentration of the sKL compound is about 50, 60, 70, 75, 80, 85, 90, 95, 100, 110, 120, 130, 140, 150, 160 170, 180, 190, 200, 210, 220, 230, 240, 250, 260, 270, 280, 290, 300, 310, 320, 330, 340, 350, 360, 370, 380, 390, 400, 500, 600, 700, 800, 900, or 1000 nM. In some embodiments of the method the concentration of the sKL compound is at least 50, 60, 70, 75, 80, 85, 90, 95, 100, 110, 120, 130, 140, 150, 160 170, 180, 190, 200, 210, 220, 230, 240, 250, 260, 270, 280, 290, 300, 310, 320, 330, 340, 350, 360, 370, 380, 390, 400, 500, 600, 700, 800, 900, or 1000 nM. In some embodiments of the method the concentration of the sKL compound is at most 50, 60, 70, 75, 80, 85, 90, 95, 100, 110, 120, 130, 140, 150, 160 170, 180, 190, 200, 210, 220, 230, 240, 250, 260, 270, 280, 290, 300, 310, 320, 330, 340, 350, 360, 370, 380, 390, 400, 500, 600, 700, 800, 900, or 1000 nM. Various embodiments of the method may contain a range of active agent between any one of the minima disclosed above and any one of the maxima disclosed above, with the caveat that the minimum must be lower than the maximum in the range. In a preferred embodiment the concentration of the sKL compound is above 75 nM. In a further preferred embodiment the concentration of the sKL compound is at least 150 nM. In a yet further preferred embodiment the concentration of the sKL compound is at least 300 nM.

The sKL compound can be used to reduce hypertrophy in a cardiac myocyte. A method of modulating reducing hypertrophy is a cardiac myocyte is provided comprising contacting the myocyte with any of the sKL compounds disclosed above. In some embodiments of the method, the myocyte is contacted with the sKL in vitro, such as in cell culture or in an organ or tissue sample that has been isolated from the animal. In some embodiments of the method, the myocyte is found in an organism, such as an animal subject. In such embodiments the method comprises administering the sKL compound to the subject in an amount effective to reduce hypertrophy in the myocyte. In some embodiments of the method the hypertrophy is FGF23-induced hypertrophy. The hypertrophy may be measured by any suitable means, such as cell area. The concentration of the sKL compound in contact with the myocyte will be sufficient to accomplish said reduction in hypertrophy, such as a decrease in observed hypertrophy compared to the absence of the sKL compound. In some embodiments of the method the concentration of the sKL compound is 1-1000 ng/ml. Further embodiments of the method involve a concentration of sKL that is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 ng/ml. Further embodiments of the method involve a concentration of sKL that is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 ng/ml. Further embodiments of the method involve a concentration of sKL that is at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 ng/ml. Various embodiments of the method may involve a range of sKL compound between any one of the minima disclosed above and any one of the maxima disclosed above, with the caveat that the minimum must be lower than the maximum in the range. In a preferred embodiment the concentration is 100 ng/ml. In those embodiments in which the sKL compound is administered to a subject to modulate the hypertrophy, it may be administered in an amount sufficient to achieve any of the concentrations disclosed above.

In the foregoing discussion “animal subject” refers to any member of Kingdom Animalia, not to exclude H. sapiens.

### Nucleic Acids

Nucleic acid molecules are provided that encode any of the sKL compounds described above. These include a nucleic acid comprising a sequence having at least a certain level of identity with any one of SEQ ID NO: 1-10. The level of identity may be ≥50%, 60%, 70%, 75%, 80%, 85%, 90%, 95%, 99%, and 100%. In a specific embodiment the level of identity is >95%. The nucleic acids also include those that anneal under stringent conditions with any of the nucleic acid molecules that encode the sKL compounds. Some embodiments of the nucleic acid anneal to any of the foregoing under highly stringent conditions. In some further embodiments of the nucleic acid, the nucleic acid anneals to any of the foregoing under maximally stringent conditions. In a specific embodiment of the nucleic acid, the nucleic acid is the exact complement to any of the foregoing nucleic acids. These molecules may be any type of nucleic acid, including RNA, DNA, LNA, BNA, copolymers of any of the foregoing, and analogs thereof. In a specific embodiment, the nucleic acid is DNA.

Stringency is based on the melting temperature (Tₘ) of the nucleic acid binding complex, as taught in Berger and Kimmel (1987, Guide to Molecular Cloning Techniques, Methods in Enzymology, 152, Academic Press, San Diego CA-incorporated herein by reference to the extent necessary to enable what is claimed). The Tₘ of an annealed duplex depends on the base composition of the duplex, the frequency of base mismatches, and the ionic strength of the reaction medium. The Tₘ of a duplex can be calculated based on these two factors using accepted algorithms. Maximum stringency typically occurs at about 5° C. below Tₘ; high stringency at about 5-10° C. below Tₘ; intermediate stringency at about 10-20° C. below Tₘ; and low stringency at about 20-25° C. below Tₘ. A maximum stringency hybridization can be used to identify or detect identical nucleotide sequences while an intermediate (or low) stringency hybridization can be used to identify or detect similar or related sequences. The term “stringent” by itself in this context refers to intermediate stringency. Terms such as maximally stringent, highly stringent, and poorly stringent, refer to conditions of maximal stringency, high stringency, and low stringency respectively.

Organisms and vectors comprising any of the nucleic acids above are also provided. Examples of uses for such organisms and vectors are production of the nucleic acids, cloning of the nucleic acids, and stable storage of the same. Many suitable vectors are known in the art, such as viruses, plasmids, cosmids, fosmids, phagmids, artificial chromosomes, yeast artificial chromosomes, human artificial chromosomes, plant transformation vectors, and liposomes. Unicellular organisms are particularly useful in cloning, replicating, and maintaining nucleic acids of interest. Model unicellular organisms that are commonly used for this purpose include yeasts, other fungi, bacteria, protists, and archaea. Specific model organisms are well known in the art, and include bacteria such as Escherichia coli, Salmonella typhimurium, Pseudomonas fluorescens, Bacillus subtilis, Mycoplasma genitalium, and various Synechocystis sp.; protists such as Dictyostelium discoideum, Tetrahymena thermophila, Emiliania huxleyi, and Thalassiosira pseudonana; and fungi such as Aspergillus sp., Neurospora crassa, Saccharomyces cerevisiae, and Schizosaccharomyces pombe.

A genetically modified (GM) organism is provided that contains any of the nucleic acids described above that encode the sKL compound, or are complementary to those that encode the sKL compound. The term “genetically modified” in this context means that genetic material has been altered by human intervention. In the context of a self-replicating entity, such alteration may have been performed on the self-replicating entity in question, or on an ancestor of the sell-replicating entity from whom it acquired the alteration. In some embodiments of the GM organism the nucleic acid is heterologous (of a different species). In further embodiments of the GM organism the nucleic acid comprises a mutation compared to wild-type.

In some embodiments of the GM organism the nucleic acid is operably linked to one or more regulatory elements. Examples of such regulatory elements include promoters, enhancers, silencers, and insulators. The regulatory element may be native to the organism, or heterologous, as necessary to accomplish the mode of expression desired.

The GM organism may be an animal that expresses the sKL. The animal may be any described above as a suitable “subject.” Some embodiments of the GM animal are a non-human animal. The non-human animal excludes H. sapiens, and any other species that is interpreted to be “human” (e.g., other extinct species of the genus Homo).

A method of making an sKL compound is provided, comprising expressing a nucleic acid that encodes any of the sKL compounds described above. The nucleic acid may be in the milieu of a GM organism as described above. The GM organism may be a microorganism that is cultured to produce the sKL compound. Culturing may occur in the presence of a suitable carbon source and a suitable energy source, depending on the organism. Some embodiments of the culture medium contain glucose, which is widely used by heterotrophic organisms as both a source of carbon and energy. The glucose can be part of a defined medium or part of a complex medium. Numerous such glucose media are known in the art, some of which are cataloged in R. Atlas, Handbook of Microbiological Media, Fourth Edition, CRC Press (2010). The sKL compound may also be made by ex vivo methods of protein synthesis as well.

### WORKING EXAMPLES

The terms “we” and “our” used below refer to authors of the write-ups of the work described, and persons who contributed to the work, some of whom might not be considered “inventors” of what is claimed under the laws of certain countries; the use of such terms is neither a representation nor an admission that any person is legally an inventor of what is claimed.

### Introduction

Here, it is investigated whether soluble klotho, a circulating protein with cardio-protective properties, and heparin, a factor that is routinely infused into patients with kidney failure during the hemodialysis procedure, regulate FGF23/FGFR4 signaling and effects in cardiac myocytes. A plate-based binding assay was developed to quantify affinities of specific FGF23/FGFR interactions and found that soluble klotho and heparin mediate FGF23 binding to distinct FGFR isoforms. Heparin specifically mediated FGF23 binding to FGFR4 and increased FGF23 stimulatory effects on hypertrophic growth and contractility in isolated cardiac myocytes. When repetitively injected into two different mouse models with elevated serum FGF23 levels, heparin aggravated cardiac hypertrophy. A novel procedure for the synthesis and purification of recombinant soluble klotho was also developed, which showed anti-hypertrophic effects in FGF23-treated cardiac myocytes. Thus, soluble klotho and heparin act as independent FGF23 co-receptors with opposite effects on the pathologic actions of FGF23, with soluble klotho reducing and heparin increasing FGF23-induced cardiac hypertrophy. Heparin injections during hemodialysis in patients with extremely high serum FGF23 levels may contribute to their high rates of cardiovascular events and mortality.

The experimental studies herein show that heparin acts as a cofactor that increases FGF23's binding affinity for the cardiac FGF23 receptor and thereby aggravates the pathologic actions of FGF23 on the heart. Hemodialysis does not reduce the cardiovascular risk of patients with end-stage renal disease, and future clinical studies should test whether frequent heparin infusions contribute to the extremely high mortality rates in these patients and whether removal of heparin from the dialysis process might improve cardiac outcomes and survival.

The studies herein suggest that pharmacologic FGFR4 blockade might serve as a novel therapeutic option to prevent or treat pathologic cardiac remodeling in CKD (also called uremic cardiomyopathy). Such novel mechanistic findings might be of high clinical relevance, as CKD is a public health epidemic that affects approximately 26 million Americans and many more individuals worldwide. 18 The presence of CKD increases the risk of premature death, and cardiovascular disease is the leading cause at all stages of CKD. 19-20 Cardiac hypertrophy is an important mechanism of cardiovascular injury in CKD that contributes to diastolic dysfunction, heart failure, arrhythmia, and sudden death.²¹ Cardiac hypertrophy affects 50% to 70% of patients during intermediate stages of CKD and up to 90% by the time they reach dialysis. 22-24 The molecular pathways responsible for cardiac hypertrophy in CKD remain uncertain, and existing treatments improve outcomes only modestly.²⁵⁻²⁸ For example, hypertension is common in CKD, and it has been hypothesized that cardiac hypertrophy develops as a result of pressure overload.²⁹ However, the correction of hypertension in animal models with kidney injury does not prevent hypertrophy.³⁰⁻³²

FGF23 is a member of the FGF family that comprises 18 secreted proteins that have diverse functions in development and metabolism.³⁵ The biological effects of all FGFs are mediated by binding to 1 of the 4 FGFR isoforms (FGFR1-4) that belong to the superfamily of receptor tyrosine kinases. 36 Alternative splicing in the juxtamembrane region of FGFR1 to 3 produces b and c variants with different biological impacts on the basis of their distinct ligand-binding spectrum. Paracrine FGFs, such as FGF1 and FGF2, bind heparan sulfate (HS), which functions as an FGFR coreceptor.³⁶ HS promotes the formation of a stable FGF/FGFR/HS ternary complex as well as FGFR dimerization, leading to the formation of a symmetric signaling complex.³⁷⁻³⁸ Compared with paracrine FGFs, the subfamily of endocrine FGFs, that is, FGF19, FGF21, and FGF23, have intrinsically low binding affinity for HS.³⁹-42 Instead of HS, endocrine FGFs use a family of singlepass transmembrane proteins, known as klotho for FGF23 and β-klotho for FGF19 and FGF21, to promote efficient FGFR binding on specific cell types.⁴¹

Klotho is a single-pass transmembrane protein that is mainly expressed in the kidney.^{3,43,44} The ectodomain of klotho can be proteolytically cleaved, which generates a soluble fragment (sKL) that can be released from the kidney and detected in the circulation.⁴⁵⁻⁴⁹ It was postulated that sKL functions as a hormone with pleiotropic, tissue-protective effects, 50-51 which acts independently of FGF23.

The recent analysis of the crystal structure revealed that FGF23, sKL, and FGFR1 c form a complex, where sKL functions as a nonenzymatic scaffold protein that brings FGFR1c and FGF23 in close proximity, thereby conferring stability of the ternary complex and promoting FGF23-mediated signaling.⁶⁵ The study herein suggests that sKL acts as an on-demand bona fide coreceptor for FGF23 and that the effects of sKL are FGF23 dependent, thereby challenging the concept that sKL can function as an FGF23-independent hormone. Surprisingly, this structural study also reveals the presence of HS in the FGF23/FGFR1c/sKL complex. 65 sKL and HS are part of the FGF23/FGFR signaling complex, and herein the order of binding events among the different components is studied. It is determined whether sKL and HS function as independent FGF23 coreceptors and act on different FGFR isoforms, and it is analyzed as to whether both cofactors modulate the pathologic FGFR4-mediated actions of FGF23 on the heart.

### Methods

Mouse soluble and full-length klotho and β-klotho cDNAs were kindly provided by Dr. Makoto Kuro-o (University of Texas Southwestern Medical Center, Dallas, TX),^{43,66} and human soluble and full-length klotho cDNA constructs were from Amgen.⁶⁷ Inserts were subcloned into vectors with appropriate epitope tags, that is, C-terminal Strep/6×His (OG1240, Oxford Genetics) or C-terminal FLAG/His (OG1232, Oxford Genetics). Subcloning was performed with appropriate restriction enzymes (New England Biolabs [NEB]) according to the manufacturer's protocols, and constructs were verified by Sanger sequencing. The EGFP-N1 vector used for green fluorescent protein (GFP) overexpression was from Clontech.

Recombinant proteins from R&D Systems are human FGF2 (233-FB/CF), human FGF5 (237-F5/CF), human FGF7 (251-KG/CF), human FGF8b (423-F8/CF), human FGF19 (969FG025/CF), human FGF21 (2539FG025/CF), human FGF23 (2604FG025/CF), mouse FGF23 (2629FG025/CF), human FGFR1c (658FR050), human FGFR2c (712FR050), human FGFR3c (766FR050), and human FGFR4 (685FR050). The sKL recombinant protein produced is mouse aa 35-982 and human aa 34-981. Mouse sKL from R&D Systems (1819KL050) is aa 35-982; human klotho domain 1 (KL1) from PeproTech is aa 34-549. Heparin solution is from Celsus Laboratories for surface plasmon resonance experiments and from Pfizer Injectables for all other studies (NDC0069005902). Primary antibodies used are anti-FGF19 (AF969, R&D Systems), anti-FGF21 (AF2539, R&D Systems), anti-FGF23 (AF2604, R&D Systems), anti-FLAG (F1804, Sigma-Aldrich), anti-human crystallizable fragment (Fc; W4031, Promega), total extracellular signal-regulated kinase (ERK; 4695S, Cell Signaling), phosphorylated ERK (9101S, Cell Signaling), glyceraldehyde-3-phosphate dehydrogenase (GAPDH; CB1001, Millipore), and sarcomeric α-actinin (EA-53, Sigma-Aldrich). Secondary antibodies are horseradish peroxidase (HRP)-conjugated anti-goat (V8051, Promega), anti-mouse (W4021, Promega), and anti-rabbit (W4011, Promega) for Western blotting; HRP-coupled anti-human antibody (109035098, Jackson ImmunoResearch Laboratories) and anti-FLAG (A8592, Sigma-Aldrich) for the plate-based binding assay; and Cy3-conjugated goat-anti-mouse (115165166, Jackson ImmunoResearch Laboratories) for immunocytochemistry. Anti-FGF23 antibody was used and provided by Amgen to block FGF23 effects in vitro.⁶⁸

To overexpress and purify recombinant sKL protein, Expi-human embryonic kidney (HEK) 293 cells (Expi293; A14527, Thermo Fisher Scientific) were used that were adapted to attached growth in Dulbecco's Modified Eagle's Medium (DMEM; 10013CV, Corning) supplemented with 10% fetal bovine serum (FBS; 26140079, Gibco) and 1× Penicillin-Streptomycin (Pen/Strep; 15140122, Gibco). Cells were transfected with appropriate klotho cDNA vectors using FuGENE 6 (E2691, Promega) in Opti-MEM (31985062, Gibco). After 2 days, clones were selected by using 1 μg/ml of puromycin (A1113803, Gibco). The clone with the highest expression was selected and readapted to suspension growth in Expi293 Expression Medium (A1435101, Thermo Fisher Scientific) supplemented with 1 μg/ml of puromycin and 1× Pen/Strep. Cells were grown in 250-ml shaker flasks with 100 ml of the medium on the MaxQ CO₂ plus shaker (88881101, Thermo Fisher Scientific) at 14 g, 37° C., and 5% CO₂. For protein production, cells were allowed to reach maximum density and collected via centrifugation at 400 g and reseeded at densities of 1:100. Cells from 500 ml of the medium were pooled and lysed in 150 ml of radioimmunoprecipitation assay buffer (RIPA; 50 mM sodium phosphate [pH 7.5], 200 mM NaCl, 1% Triton X-100, 0.25% deoxycholic acid) with the addition of a protease inhibitor (11873580001, Roche) for 30 minutes. The mixture was then centrifuged at 20,000 g for 1 hour to remove all cell debris, and the supernatant was sterile filtered for purification. Lysate was applied to a 5 ml TALON column (28953767, GE Healthcare) on the AKTA Start system (GE Healthcare) in running buffer containing 50 mM sodium phosphate and 300 mM NaCl. Elution was performed in running buffer supplemented with 200 mM imidazole.

For Strep-tagged sKL, fractions positive for sKL protein were diluted 1:2 in Buffer W (100 mM tris(hydroxymethyl)-aminomethane [Tris] [pH 8.0], 150 mM NaCl), captured on a 1-ml Strep-Tactin XT 4Flow column (24025001, IBA Lifesciences), and eluted with 100 mM biotin (BP2321, Fisher) in Buffer W. Positive fractions were collected, diluted 1:10 in 25 mM Tris (pH 8.0) running buffer, and applied to a 1 ml HiTrap Q HP column (29051325, GE Healthcare). The sample was eluted with running buffer in 500 mM NaCl, and sKL was aliquoted and flash frozen. The protein concentration was determined using Coomassie gel analysis in comparison to a bovine serum albumin (BSA) gradient.

For FLAG-tagged sKL, TALON column-positive fractions were diluted 1:10 in 10 mM Tris (pH 7.0) running buffer and applied to a 5 ml heparin column (17040701, GE Healthcare). The sample was eluted on a linear gradient of 0.0 to 1.0 M NaCl. Positive fractions for sKL were identified via a plate-based detection assay (detailed below) and pooled. The sample was diluted 1:10 and applied to a 1 ml HiTrap Q HP column (29051325, GE Healthcare) after TALON column purification. The sample was eluted with a 0.0 to 0.5 M NaCl gradient. Again, positive sKL fractions were identified by a klotho detection assay and flash frozen. The protein concentration was determined using Coomassie gel analysis in comparison to a BSA gradient.

To study the interaction between FLAG-tagged sKL and different FGF isoforms, co-immunoprecipitation studies were conducted. FLAG beads (A2220, Sigma-Aldrich) were used at 50 ul of 50% stock slurry per sample. Beads were washed 5× with activity buffer (50 mM Tris [pH 7.4], 200 mM NaCl, 0.01% Tween 20, Sigma-Aldrich). Bead spin-down assays were all performed for 2 minutes at 5000 g. Fifty microliters of a 50% slurry of beads per sample was resuspended in 1 ml of activity buffer in a 1.5-ml snap cap tube. One microgram of FLAG-tagged sKL or Carboxy-terminal bacterial alkaline phosphatase (BAPC; P7457, Sigma-Aldrich) was incubated with the beads for 1 hour on a bead rotator. Beads were then pelleted at 5000 g for 2 minutes and washed 5× with activity buffer. Beads were resuspended in 1 ml of activity buffer and samples with 500 ng of FGF. Beads were incubated for 1 hour on a tube rotator, and beads were pelleted and washed as before. One hundred microliters of I× Laemmli sample buffer (1610737, Bio-Rad) with 1.42 M 2-mercaptoethanol was added, and samples were boiled for 5 minutes and analyzed using sodium dodecylsulfate-polyacrylamide gel electrophoresis (SDS-PAGE) and Western blotting, as described below herein.

To study the interaction between Fc-tagged FGFR isoforms (Fc-FGFR), FGF isoforms, and sKL, immunoprecipitation studies were conducted. Protein A beads (6501, BioVision) and protein G beads (6511, BioVision) were used at 25 μl of a 50% stock slurry per sample in a total bead volume of 50 μl. Beads were washed 5× in activity buffer (50 mM Tris [pH 7.4], 200 mM NaCl, 0.01% Tween 20). Bead spin-down assays were all performed for 2 minutes at 5000 g. Beads were resuspended in 1 ml of activity buffer in a 1.5-ml snap cap tube. One microgram of Fc-FGFR1 to 4 was incubated with the beads for 1 hour on a bead rotator. Control samples were incubated with 2 μg of anti-FGF antibody instead of Fc-FGFR. Beads were then pelleted at 5000 g for 2 minutes and washed 5× with activity buffer. Beads were resuspended and incubated with different combinations of 500 ng of FGF, 1 μg of sKL, 1.6 United States Pharmacopeia (USP) units of heparin, or phosphate-buffered saline (PBS). Beads were incubated for 1 hour on a tube rotator, and beads were pelleted and washed as before. One hundred microliters of 1× Laemmli sample buffer (1610737, Bio-Rad) with 1.42 M 2-mercaptoethanol was added, and samples were boiled for 5 minutes. Samples were analyzed using SDS-PAGE and Western blotting.

The sample (20 μl) was loaded onto Mini-PROTEAN TGX Precast Gel 4-20% for experiments with <9 samples (4568094, Bio-Rad) or Criterion TGX Precast Gel 4-20% (5678094, Bio-Rad) for experiments with >9 samples. Gels were run in 1× Tris/Glycine/SDS buffer (1610732, Bio-Rad) at 150 V and stopped when the sample dye reached the end of the gel. Gels were removed and electrotransferred via a semi-dry cassette (1703940, Bio-Rad) in I× Tris/Glycine buffer (1610734, Bio-Rad) with 20% methanol. Gels were transferred onto polyvinylidene diflouride membranes (IPVH00010, Millipore) at 20 V for 1 hour. Membranes were then blocked in 5% dried milk with 0.5% Tween 20 diluted in Tris-buffered saline (TBST; pH 7.5) for 1 hour and probed with antibodies against specific antigens at 1:1000 in TBST with 5% dried milk. Blots were washed 3× for 5 minutes in TBST and probed with HRP-coupled secondary antibodies against specific species at 1:10,000 in TBST with 5% dried milk. Membranes were imaged on the SRX-101A X-ray film developer (Konica Minolta).

To determine the cellular activity of paracrine FGFs, the activation/phosphorylation of ERK in HEK293 cells after treatment was studied. HEK293 cells were seeded in 6-well plates in DMEM (26140079, Gibco) supplemented with I× Pen/Strep (15140122, Gibco) and 10% FBS (26140079, Gibco) and grown for 48 hours. Upon reaching 80% confluence, cells were serum starved overnight in DMEM and 1× Pen/Strep without FBS. The medium was changed, and cells were treated with sKL at 2 μg/ml or vehicle (sKL elution buffer: 25 mM Tris ph 8.0, 500 mM NaCl) for 15 minutes. Cells were then treated with various FGFs at 1.1 nM for 10 minutes. Cells were lysed in 200 μl of RIPA with the addition of a protease inhibitor (11836153001, Roche) and phosphatase inhibitors (P5726 and P0044, Sigma-Aldrich) for 30 minutes. The mixture was then centrifuged at 20,000 g for 60 minutes to remove all cell debris. One hundred microliters of 1× Laemmli sample buffer (1610737, Bio-Rad) with 1.42 M 2-mercaptoethanol was added, and samples were boiled for 5 minutes. Twenty microliters of samples was loaded onto 12% SDS-PAGE gels and analyzed using Western blotting for total and phosphorylated ERK.

Proteins were coated on 96-well plates (MaxiSorp; 439454, Thermo Fisher Scientific), and proteins were added to wells in 100 μl of enzyme-linked immunosorbent assay (ELISA) coating buffer (E107, Bethyl Laboratories) with a lid placed overtop and incubated at 4° C. overnight. Plates were washed 5× with 350 μl of assay buffer (50 mM Tris [pH 7.4], 200 mM NaCl, 0.01% Tween 20) on a 50 TS microplate washer (BioTek). Plates were blocked for 1 hour in 200 μl of assay buffer with 0.5% BSA. Plates were washed as before, and proteins were incubated on plates in 100 μl of assay buffer with 0.5% BSA. Proteins or heparin was incubated in orders described in the respective figure legends. Assays using FLAG-tag-based detection were incubated with anti-FLAG coupled to HRP at 1:20,000, and assays using Fc detection were incubated with anti-human Fc coupled to HRP at 1:10,000. Plates were washed as above, and 100 μl of 3,3′,5,5′-tetramethylbenzidine (TMB) substrate (E102, Bethyl Laboratories) was added for 15 to 20 minutes until positive wells developed a dark blue color. Reactions were stopped with ELISA stop solution (E115, Bethyl Laboratories) and analyzed using the Synergy HI plate reader (BioTek) at 450 nm wavelength. All samples were run in triplicates, and individual readouts are displayed on graphs. As a control to remove background absorbance values, wells were coated with an equal amount of BSA to match the amount of protein coated in experimental wells and treated the same as experimental wells. To study the effects of sKL on the binding between paracrine FGFs and FGFRs, plates were run as above except coated with paracrine FGFs. Fc-FGFR1 was preincubated with sKL or vehicle for 60 minutes, and then the combination was added to wells and the assay was run as before.

To determine the half-life of recombinant sKL protein in the circulation, rats were injected with the Strep-tagged mouse sKL that was purified as described above. Each group consisted of 3 male Sprague Dawley rats (9 weeks of age and weighing approximately 300 g). For injections and bleeding, rats were anesthetized with 2.5% isoflurane. For injections, a shielded i.v. catheter (BD Insyte Autoguard 0.7×19 mm, catalog no. 381412) was inserted into the lateral tail vein and flushed with 100 μl of isotonic saline (0.9%). One milliliter of purified sKL protein dissolved in isotonic saline (100 μg/kg) or isotonic saline was then injected, and the catheter was flushed again with 100 μl of isotonic saline. For bleeding, a shielded i.v. catheter was inserted into the lateral tail vein. Blood samples (300 μl) were taken after 1, 3, 5, 10, 15, 30, and 60 minutes as well as 3, 6, 12, and 48 hours. For collections from 15 minutes to 48 hours postinjection, a separate i.v. catheter was inserted each time to draw blood. For collections from 1 to 15 minutes post-sKL injection, the same i.v. catheter was used, and to exclude contamination with the previous draw, the catheter was flushed with isotonic saline between each sample. Blood was collected into Multivette serum gel tubes (15.16.74, Sarstedt) and centrifuged at 10,000 g for 5 minutes to purify serum. Serum was then aliquoted and stored at approximately 80° C. before analysis. For the sKL activity assay, wells were coated with 500 ng of mouse FGF23 in 100 μl of coating buffer (E107, Bethyl Laboratories) at 4° C. overnight. Plates were washed 5× at 350 μl of assay buffer (50 mM Tris, [pH 7.4] 200 mM NaCl, 0.01% Tween 20) on a 50 TS microplate washer (BioTek). Plates were blocked for 1 hour in 200 μl of assay buffer with 0.5% BSA. Plates were washed and incubated with 30 μl per well and 70 μl of assay buffer with 0.5% BSA. Standards were run at noted concentrations by using purified Strep-tagged mouse sKL protein with 30 μl of control serum and 70 μl of assay buffer with 0.5% BSA added to ensure consistency. All samples and standards were run in triplicates. After 1 hour, plates were washed, and 150 ng of Fc-FGFR1c were incubated on plates in 100 μl of assay buffer with 0.5% BSA. After 1 hour, plates were washed and incubated with anti-human Fc-HRP at 1:10,000. Plates were washed as above and 100 μl of TMB substrate (E102, Bethyl Laboratories) was added for 15 to 20 minutes until positive wells developed a dark blue color. Reactions were stopped with ELISA stop solution (E115, Bethyl Laboratories) and analyzed using the Synergy HI plate reader (BioTek) at 450 nm wavelength. Amounts of sKL were calculated by taking the slope of the standard curve and calculating where the sample points fit in.

HEK293 cells were plated in 10 cm dishes supplemented with 10% FBS (26140079, Gibco) and I× Pen/Strep (15140122, Gibco). Cells were transfected with appropriate klotho or green fluorescent protein cDNA constructs. After 2 days, clones were selected by using 1 μg/ml of puromycin (A1113803, Gibco). Cells were then split and plated in 10 cm dishes in DMEM supplemented with FBS and puromycin. After 48 hours, cells were lysed in 1 ml of RIPA buffer with the addition of a protease inhibitor (11873580001, Roche) for 30 minutes. The mixture was then centrifuged at 20,000 g for 1 hour to remove all cell debris. Activity assay was run as above in the sKL halflife study. Instead of serum, 20 μl of the indicated cell lysate was used and combined with 80 μl of activity assay buffer. All other conditions were as listed above and data displayed as absorbance after 15 to 20 minutes.

For the direct quantitative analysis of label-free molecular interactions in real time, surface plasmon resonance studies were conducted using BIAcore T200 and T200 evaluation software (28975001, Cytiva). Sensor SA (BR100398) and CMS chips (BR100399) were from Cytiva. For the preparation of heparin chips, heparin (2 mg) and amine-PEG3-Biotin (2 mg, 21347, Pierce) were dissolved in 200 μl of H₂O and 10 mg of NaCNBH₃ was added. The reaction mixture was heated at 70° C. for 24 hours; an additional 10 mg of NaCNBH₃ was added; and the reaction was heated at 70° C. for another 24 hours. After cooling to room temperature, the mixture was desalted with the spin column (3000 MWCO). Biotinylated heparin was collected, freeze dried, and used for chip preparation. Biotinylated heparin was immobilized to streptavidin chips according to the manufacturer's protocol. In brief, 20 μl of biotinylated heparin solution (0.1 mg/ml) in HBS-P running buffer (0.01 M N-2-hydroxyethylpiperazine-N′-2-ethanesulfonic acid, 0.15 M NaCl, 0.005% surfactant P20 [pH7.4]; BR100368, Cytiva) was injected over flow cell 2 of the streptavidin chip at a flow rate of 10 μl/min. The successful immobilization of heparin was confirmed by the observation of an approximately 200-resonance unit increase in the sensor chip. The control flow cell (flow cell 1) was prepared by 1-minute injection with saturated biotin. For the preparation of FGFR4 chips, Fc-FGFR4 protein (human ectodomain) was immobilized on research grade CMS chips according to the standard amine coupling protocol. Briefly, carboxymethyl groups on the CMS chip surface were first activated using an injection pulse of 35 ml (flow rate 5 ml/min) of an equimolar mix of N-ethyl-N-(dimethylaminopropyl) carbodiimide and N-hydroxysuccinimide (final concentration 0.05 M, mixed immediately before injection). After activation, FGFR4 was diluted to 50 g/ml in 100 mM sodium acetate (pH 5.5) buffer and injected over the activated biosensor surface. Excess unreacted sites on the sensor surface were deactivated with a 35 μl injection of 1 M ethanolamine. A reference flow cell was prepared using an injection pulse of 35 ml (flow rate 5 ml/min) of an equimolar mix of N-ethyl-N-(dimethylaminopropyl) carbodiimide and N-hydroxysuccinimide (final concentration 0.05 M, mixed immediately before injection) followed by a 35 μl injection of 1 M ethanolamine. For the measurement of interactions between FGF23 (human protein), FGFR4 (human ectodomain protein), klotho (human ectodomain, self-made; see above), and heparin, samples were diluted in HBS-P buffer. Different dilutions of protein were injected at a flow rate of 30 μl/min with HBS-P buffer as running buffer. At the end of the sample injection, HBS-P buffer was flowed over the sensor surface to facilitate dissociation. After a 3-minute dissociation time, the sensor surface was regenerated by injecting with 30 μl of 2 M NaCl to get a fully regenerated surface. The response was monitored as a function of time (sensorgram) at 25° C.

Neonatal rat ventricular myocytes (NRVMs) were isolated using a kit according to the manufacturer's protocol (LK003300, Worthington Biochemical Corporation), as done previously.^{13,16,17} Briefly, hearts from 1- to 3-day-old Sprague Dawley rats (Envigo) were harvested and minced in calcium- and magnesium-free Hanks balanced salt solution and the tissue was digested with 50 μg/ml of trypsin at 4° C. for 20 to 24 hours. Soybean trypsin inhibitor in Hanks balanced salt solution was added, and the tissue was further digested with collagenase (in Leibovitz L-15 medium) under slow rotation (0.14 g) at 37° C. for 45 minutes. Cells were broken up by triturating the suspension 20× with a standard 10-ml plastic serological pipette. Cells were then filtered through a cell strainer (70 μm, BD Falcon). Cells were incubated at room temperature for 20 minutes and spun at 100 g for 5 minutes. The cell pellet was resuspended in plating medium (DMEM Base [10013CV, Corning], 17% M199 [12350039, Gibco], 15% FBS [26140079, Gibco], I× Pen/Strep [15140122, Gibco]). For cell tracings, NRVMs were grown in 24-well plates on glass coverslips (CLS1760012, Chemglass). Slips were precoated with laminin (23017015, Invitrogen) at 10 μg/ml for 1 hour at 37° C. dissolved in PBS. Laminin was aspirated, and cells and medium were added. Cells were counted by a hemocytometer and plated at 4×10⁵ cells per well. Myocytes were grown for 72 hours in the presence of plating medium; the medium was removed, and maintenance medium (DMEM Base [10013CV, Corning], 20% M199 [12350039, Gibco], 1% insulin-transferrin-sodium selenite solution [118841VL, Sigma-Aldrich], I× Pen/Strep [15140122, Gibco], 100 UM 5-bromo-2′-deoxyuridine [B9285, Sigma-Aldrich]) were added. After 48 hours, maintenance medium was replaced, and after another 48 hours, cells were ready for treatment, giving a total of 72 hours of plating medium and 96 hours of maintenance medium before treatment. Cells were then treated for 48 hours.

The medium was aspirated, and cultured NRVMs were fixed in 2% paraformaldehyde (P-6148, Sigma-Aldrich) and 4% sucrose dissolved in PBS for 5 minutes followed by permeabilization with 1% Triton X-100 dissolved in PBS for 10 minutes. Coverslips were washed 3× in PBS and blocked for 1 hour in blocking solution (2% FBS [26140079, Gibco], 2% BSA [BSA50, Rockland], 0.2% coldwater fish skin gelatin [900033, Aurion] dissolved in PBS). Blocking solution was aspirated, and 100 μl of 1:1000 α-actinin primary antibody was added for 1 hour. The primary antibody was aspirated, and coverslips were washed 3× with blocking solution and then 100 μl of Cy3-conjugated goat-anti-mouse secondary antibody diluted at 1:300 was added. After 1 hour, coverslips were washed 3× with blocking solution, dabbed dry, and mounted in ProLong™ Diamond Antifade Mountant with DAPI (P36962, Thermo Fisher Scientific) for visualization of nuclei. Immunofluorescence images were taken with a Leica DMi8 fluorescence microscope with a 63× oil immersion objective. The cross-sectional area of myocytes was measured on the basis of α-actinin-positive staining using ImageJ software (v1.53, National Institutes of Health). Each slide was from a different isolation to ensure reproducibility.

As done before,⁶⁹ adult male Wistar rats (150-200 g) were killed and hearts were quickly removed and cannulated via the ascending aorta on a Langendorff system where they were retrograde perfused and digested by type II collagenase. Hearts were perfused with calcium-free Tyrode's solution supplemented with 0.2 mmol/L of ethyleneglycol-bis-((3-aminoethylether)-N,N, N′,N′-tetraacetic acid for 3 minutes. For the digestion, hearts were perfused with Tyrode's solution containing 0.1 mmol/L of CaCl₂), 1 mg/ml of type II collagenase (Worthington), and 1 mg/ml of BSA for 3 to 4 minutes at room temperature. Cells were resuspended in Tyrode's solution containing 1 mmol/L of CaCl₂). Tyrode's solution contains 130 mM NaCl, 5.4 mM KCl, 0.4 mM NaH₂PO₄, 0.5 mM MgCl₂, 25 mM N-2-hydroxyethylpiperazine-N-2-ethanesulfonic acid, and 22 mM glucose. Adult rat ventricular myocytes (ARVMs) were loaded with the fluorescent Ca²⁺ dye Fluo-3 AM (Thermo Fisher Scientific) for cytosolic Ca²⁺ measurements. Loaded ARVMs were acutely perfused during 2 minutes with vehicle, FGF23 (10 ng/ml), or a combination of FGF23 and heparin (0.7 USP/ml). Cytosolic Ca²⁺ transients were recorded under 1 Hz of field stimulation using 2 parallel platinum electrodes. Images were obtained with confocal microscopy (Zeiss LSM 510-META microscope), using a 40× objective by scanning the cell with an argon laser every 1.54 ms. Fluo-3 AM was excited at 488 nm, and emissions were collected at >505 nm. Cell contraction was stimulated as the difference in cell length between rest and contraction (during electrical stimulation), expressed as a percentage of shortening of the cell length. Image analysis was performed with self-made routines using IDL (Research Systems) and ImageJ (v1.53) software.

Isolated heart contractility was performed as described previously. 14,70,71 Briefly, 2- to 3-month-old male CD-1 mice were anesthetized using 3% isoflurane and hearts were carefully removed and placed into oxygenated Ringer's solution composed of 140 mM NaCl, 2.0 mM KCl, 2.5 mM CaCl₂), 1.0 mM MgSO₄, 1.5 mM K₂HPO₄, 10 mM N-2-hydroxyethylpiperazine-N-2-ethanesulfonic acid, and 10 mM glucose (pH 7.4). Atria were then removed, and the intact ventricular muscle was attached to small metallic clips hung vertically from a force transducer (ADInstruments) between bipolar platinum-stimulating electrodes and suspended in 25-ml glass tissue chambers (ADInstruments). Heart muscles were perfused with Ringer's solution continuously bubbled with 100% O₂ at room temperature. Hearts were stretched to the length of maximum force development and paced at 1 Hz (5 ms pulse duration, 30-60 V; Grass Instruments SD9 Square Pulse Stimulator). After a stable baseline was obtained, hearts were treated with either vehicle or FGF23 (9 ng/ml) alone or in combination with heparin (0.06 USP/ml) while contractile force output was monitored for 30 minutes. Contractile data were recorded and analyzed with LabChart 8 software (ADInstruments). Waveform changes were analyzed for peak contraction force (in millinewtons) and maximum slope of force development and presented as fold change from baseline values.

FGF23 injections were administered following a similar protocol established previously. 13,72,73 Briefly, 12-week-old male BALB/c mice (The Jackson Laboratory) underwent tail vein injections. The day before the experiment, mice underwent echocardiographic analysis, as described below. Mice were anesthetized in 2.5% isoflurane and placed on a heat pad. Each group consisted of 5 mice, and different groups were injected with isotonic saline, heparin, FGF23, or FGF23 and heparin combined. Per injection, 40 μg/kg of FGF23 and 125, 62.5, or 12.5 USP/kg of heparin dissolved in 200 μl of isotonic saline were used, with 8 hours were used between injections for a total of 5 consecutive days. All injections were performed via the lateral tail vein. On the morning of the sixth day, 16 hours after the final tail vein injection, animals underwent echocardiographic analysis and were killed. Middle sections of hearts were cut along the short axis and stored overnight in 4% phosphate-buffered formaldehyde solution. Sections were sent to IDEXX Laboratories for embedding, hematoxylin and eosin (H&E) staining, and sectioning. Sections for wheat germ agglutinin (WGA) labeling were not stained by IDEXX Laboratories.

This diet-induced model of CKD, which develops kidney tubulointerstitial damage and cardiac hypertrophy, was constructed in accordance with previous studies.^{74,75} Male and female mice were studied in separate groups on the basis of the gender-specific effect of adenine, with females taking much longer (16-20 weeks) to develop kidney injury.⁷⁶ Five-week-old BALB/c mice (The Jackson Laboratory) were put on the control diet for 1 week. After 1 week, male and female mice were each split into 4 groups, receiving control chow and saline injections, control chow and heparin injections, adenine chow and saline injections, or adenine chow and heparin injections. Mice were administered an adenine diet (0.2% adenine, Teklad) or a control diet (same composition as adenine diet lacking adenine, Teklad) on the basis of their group and injected via the lateral tail vein with 125 USP/kg of heparin dissolved in isotonic saline or with isotonic saline as a vehicle control. Injections were administered 3 times per week, every Monday, Wednesday, and Friday, to mimic a dialysis schedule. During injections, mice were briefly anesthetized with 2.5% isoflurane and the injection was administered in the lateral tail vein. After 10 weeks, mice were killed and samples prepared as described above.

Short-axis heart sections were stained with H&E (IDEXX Laboratories) and used for representative images. Pictures were taken with a Leica Dmi8 fluorescence microscope. The crosssectional area of individual cardiac myocytes in paraffin-embedded short-axis sections was measured. Paraffin-embedded sections underwent deparaffinization 2× for 5 minutes in Shandon Xylene Substitute (9990505, Thermo Fisher Scientific) and then rehydrated through a graded ethanol series (99%, 97%, 70%), 2× for 5 minutes each. Antigen retrieval was performed in a microwave for 15 minutes in 1× unmasking solution (H3300, Vector Laboratories). Slides were washed 3× for 5 minutes each in PBS and then incubated for 1 hour in blocking solution (1% BSA50 [Rockland], 0.1% cold-water fish skin gelatin [900033, Aurion], 0.1% Tween 20). Slides were washed 3× in PBS and then incubated in 10 μg/ml of 594-conjugate WGA (W11262, Thermo Fisher Scientific) for 1 hour. Slides were washed 3× with PBS and then mounted in Prolong™ Diamond Antifade Mountant (P36961, Thermo Fisher Scientific). Immunofluorescence images were taken with a Leica Dmi8 fluorescence microscope with a 60× oil objective. ImageJ software (National Institutes of Health) was used to quantify the cross-sectional area of 25 cells per field in 4 fields along the mid-chamber free wall on the basis of WGA-positive staining.

Echocardiographic analysis was performed on day 6 of the experiment for FGF23-injected mice by using the Vevo 770 or Vevo 3100 imaging system (FUJIFILM VisualSonics). Animals were anesthetized with 1.5% isoflurane, and normal body temperature was maintained using a rectal probe for temperature monitoring. For analysis, both B- and M-mode images were obtained in the short- and long-axis views. Correct positioning of the transducer was ensured using B-mode imaging in the long-axis view before switching to the short-axis view. Image analysis was performed using Vevo 770 Workstation and Vevo LAB software (FUJIFILM VisualSonics). Measurement and calculation definitions (including formulas) for B-mode are presented in Tables A1 and A2 below.

TABLE A1
Measurement Definitions for B-Mode

| Parameter | Definition | Unit |
|---|---|---|
| Endocardial | the area defined by the inner | mm² |
| Area; d | wall of the LV in diastole. | |
| Endocardial | the area defined by the inner | mm² |
| Area; s | wall of the LV in systole. | |
| Epicardial | the area defined by the outer | mm² |
| Area; d | wall of the LV in diastole; | |
| Epicardial | the area defined by the outer | mm² |
| Area; s | wall of the LV in systole; | |
| Endocardial | the inner lengths of the major | mm |
| Major; d | axis of the LV in diastole | |

TABLE A2
Calculation Definitions for B-Mode

| Parameter | Definition | Unit | Formula |
|---|---|---|---|
| Endocardial % FAC | LV fractional area change | % | $\frac{Endocardial Area ; d - Endocardial Area ; s}{Endocardial Area ; d} \times 100$ |
| Endocardial % EF | LV ejection fraction | % | $\frac{Endocardial Stroke Volume}{Endocardial Volume ; d} \times 100$ |
| T;d | average wall thickness of the | mm | $\sqrt{\frac{Epicardial Area ; d}{π}} - \sqrt{\frac{Endocardial Area ; d}{π}}$ |
| | myocardium | | |
| LV Mass;d | the mass of LV myocardium | mg | $1.05 \times ( ( \frac{5}{6} \times Epicardial Area ; d \times ( Epicardial Major ; d + T ; d ) ) - ( \frac{5}{6} \times Endocardial Area ; d \times Endocardial Major ; d ) )$ |

At the end of the experiment, blood was collected from mice at the time of killing via cardiac puncture, transferred into Microvette serum gel tubes (20.1344, Sarstedt), and centrifuged at 10,000 g for 5 minutes. Serum supernatants were collected and stored at approximately 80° C. Analyses of standard serum chemistry parameters were performed at IDEXX BioAnalytics (60406, kidney panel); serum blood urea nitrogen was measured using mass spectrometry at the Bioanalytical Core, School of Medicine, O'Brien Center for Acute Kidney Injury Research, University of Alabama at Birmingham.

Values are expressed as mean±SEM. Comparison between groups was performed using 1-way analysis of variance followed by a post hoc Tukey test (for all comparisons of 3≥groups) or using 2-tailed t-tests. A significance level of P≤0.05 was accepted as statistically significant. No statistical method but experience from previous publications was used to predetermine sample size. No formal randomization was used in any experiment. For in vivo experiments, animals were unbiasedly assigned into different treatment groups. Group allocation was not performed in a blinded manner. Whenever possible, experimenters were blinded to the groups (e.g., in immunofluorescence and immunohistochemistry experiments by hiding group designation and genotype of animals until after quantification and analysis).

All animal protocols and experimental procedures for FGF23 injections in mice, adenine diet in mice, sKL half-life studies in rats, and NRVM isolations from newborn rats were approved by the Institutional Animal Care and Use Committee at the School of Medicine, University of Alabama at Birmingham. Heart isolations from mice for contractility studies were approved by the Institutional Animal Care and Use Committee at the University of Missouri-Kansas City. ARVM isolations from adult rats were approved by the Bioethical Committee Universidad Autonoma de Madrid and the General Direction of Agriculture and the Environment at the Environment Council of Madrid. All animals were maintained in temperature-controlled environments with a 12-hour light/dark cycle and allowed ad libitum access to food and water. All protocols adhered to the Guide for Care and Use of Laboratory Animals to minimize pain and suffering.

### Example 1

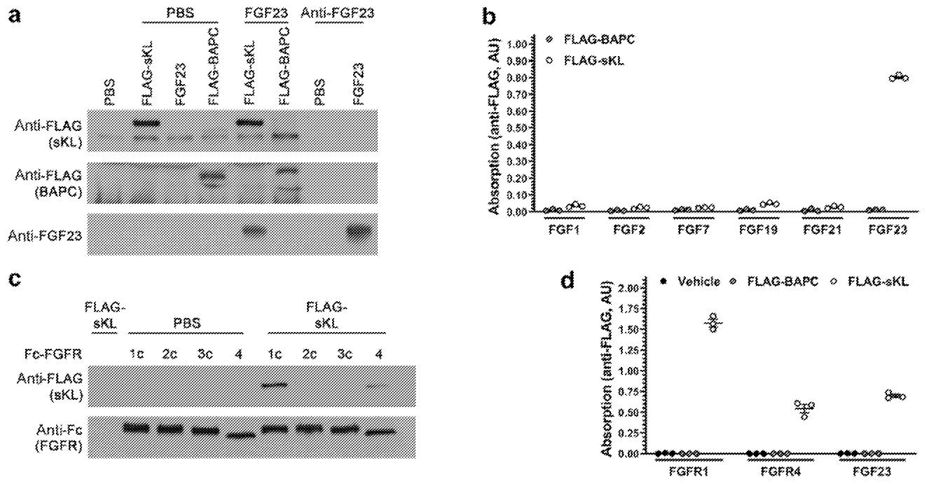

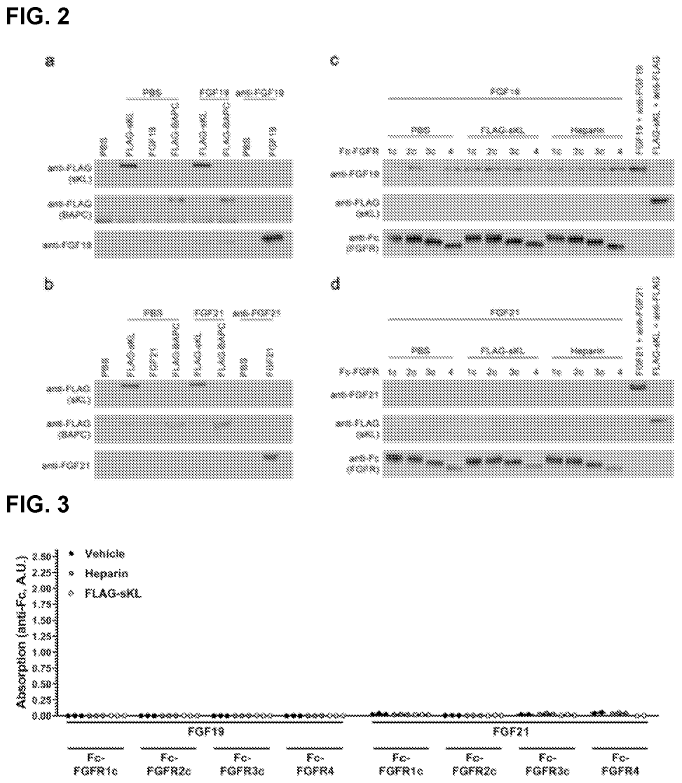

FGF23 and sKL binding was tested in the absence of FGFR. Immobilized FLAG-tagged sKL derived from transfected HEK293 cells was incubated with recombinant FGF23 protein, and FGF23 could be co-precipitated, which was not detected when a FLAG-tagged control protein was immobilized (FIG. 1A). In contrast, sKL did not bind FGF19 or FGF21 (FIG. 2A and FIG. 2B). Next, a binding assay was developed, where 96-well plates were coated with recombinant FGF23 protein, and sequentially incubated with FLAG-tagged sKL, anti-FLAG antibody, anti-Fc antibody coupled to HRP, and HRP substrate, followed by the analysis of chemiluminescence. sKL bound FGF23, which was not observed when wells were coated with other FGF isoforms (FIG. 1B). sKL binding FGFR in the absence of FGF23 was also tested. Fc-coupled FGFR ectodomains were immobilized on protein A/G beads and incubated with FLAG-tagged sKL in the absence of FGF23. FLAG-sKL could be co-precipitated with FGFR1c and to a lower extent with FGFR4 but no sKL binding to FGFR2c or FGFR3c was detected (FIG. 1C). These findings indicate that sKL can bind both FGF23 in the absence of FGFR as well as specific FGFR isoforms in the absence of FGF23. Plates were coated with FGF23, or with Fc-coupled ectodomains of FGFR1c or FGFR4, and then sequentially FLAG-tagged sKL, HRP-coupled anti-FLAG antibody, and HRP substrate were added to determine whether sKL preferentially binds FGF23 over FGFR. FLAG-sKL bound with highest affinity to FGFR1c and with similar affinities to FGF23 and FGFR4 (FIG. 1D).

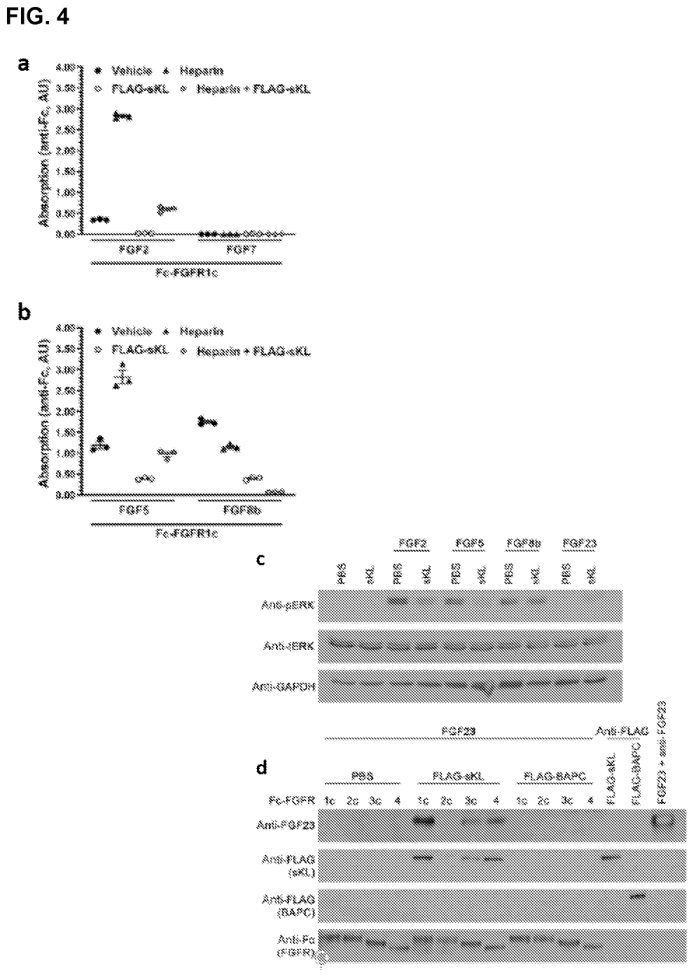

It has been shown that sKL binding to FGFRs not only enhances the affinity of FGFRs for FGF23 but concomitantly suppresses FGFR binding to paracrine FGFs.⁷⁷ A plate-based binding assay was used to further test the hypothesis that sKL acts as an inhibitor of paracrine FGFs. Wells were coated with FGF2 or FGF7 and then sequentially incubated at the same molar concentrations with Fc-FGFR1 that had been preincubated with sKL, HRP-coupled anti-Fc antibody, and HRP substrate. In the absence of sKL, FGFR1c bound FGF2, but not FGF7 (FIG. 4A), which is in line with previous studies showing that FGF7 prefers to interact with b splice isoforms of FGFRs.⁷⁷ Adding heparin before incubation with FGFR1c significantly increased FGF2 binding to FGFR1c. In contrast, heparin did not induce binding between FGF7 and FGFR1c. In the presence of FLAG-sKL, the binding of FGFR1c to FGF2 was significantly reduced. This inhibitory effect of sKL occurred in the absence and presence of heparin. Furthermore, sKL reduced the binding of FGFR1c to FGF5 and FGF8b (FIG. 4B). To determine potential functional consequences of sKL's inhibitory actions toward paracrine FGFs, HEK293 cells, which endogenously express various FGFR isoforms but lack klotho, were coincubated with FGFs and sKL. In the absence of sKL, paracrine FGF2, FGF5, and FGF8b induced ERK phosphorylation, indicating the activation of Ras/MAPK signaling, while endocrine FGF23 had no effect (FIG. 4C). Coincubation with sKL decreased phospho-ERK levels in cells treated with FGF2 and FGF5, but induced ERK phosphorylation when cells were incubated with FGF23. These findings suggest that while serving as a soluble coreceptor for FGF23 that mediates FGFR/Ras/MAPK signaling in the absence of endogenous membrane-associated klotho, sKL can block the klotho-independent activation of FGFR signaling induced by paracrine FGFs.

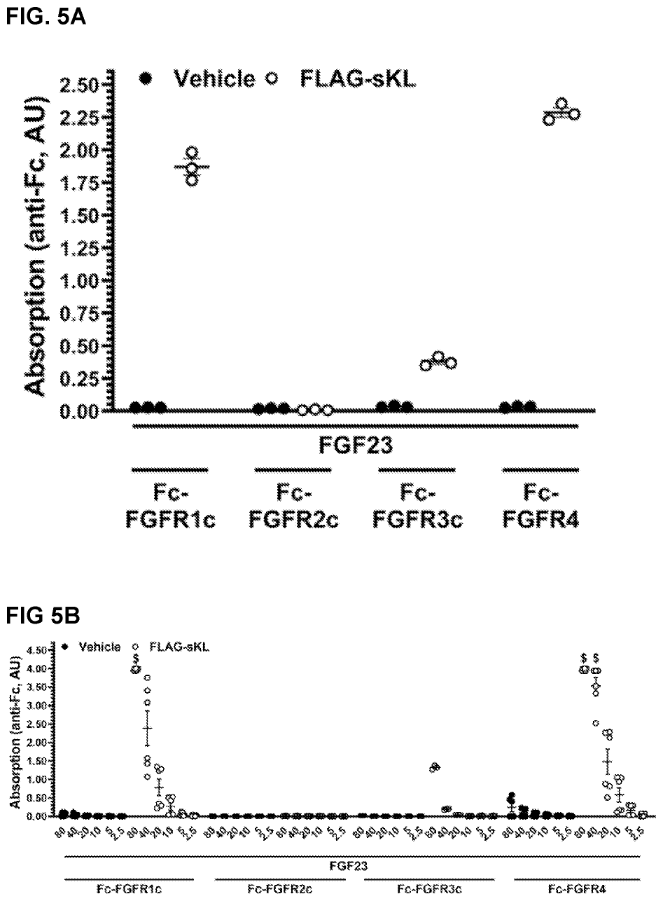

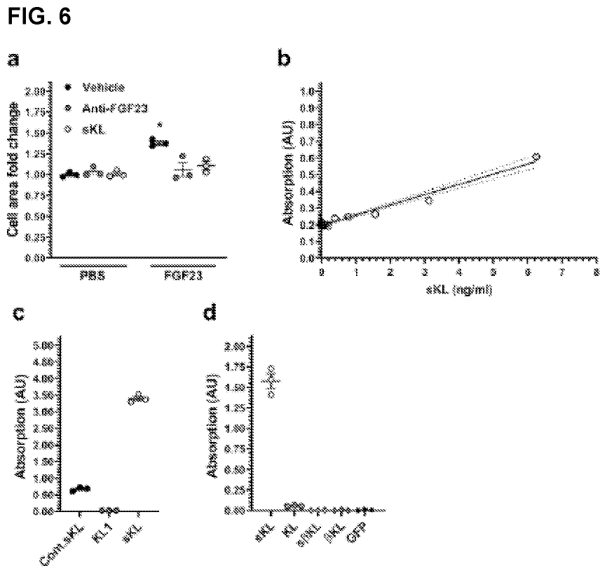

Next, the order of binding events was studied, with sKL first binding to FGFR and second to FGF23 versus sKL first binding to FGF23 and second to FGFR to determine whether either affects of FGF23's affinity for FGFRs. Fc-coupled FGFR ectodomains were immobilized on protein A/G beads, followed by coincubation with FLAG-tagged sKL and FGF23. It was found that FGFR1c, FGFR3c, and FGFR4 could coprecipitate sKL and FGF23 (FIG. 4D). In contrast, no FGF23 binding was detected to any of the FGFR isoforms in the absence of sKL. Next, plates were coated with FGF23, followed by the sequential incubation with FLAG-tagged sKL, Fc-coupled FGFR ectodomains, HRP-coupled anti-Fc antibody, and HRP substrate. In the presence of sKL, FGF23 bound to FGFR1c, FGFR3c, and FGFR4 (FIG. 5A), and these binding events occurred in a dose-dependent manner (FIG. 5B). When the plate was coated with high amounts of FGF23, FGFR4 binding was also detected in the absence of sKL, which was not detected for the other FGFR isoforms (FIG. 5B). Based on the sequential nature of the binding assay, FGF23 and sKL must have pre-formed before binding to FGFR ectodomains. As revealed by the pull-down as well as the plate binding assay, sKL did not mediate binding of FGF19 or FGF21 to any of the FGFR isoforms (FIG. 2C, FIG. 2D, and FIG. 3). These findings indicate that sKL can increase FGFR binding affinity of FGF23 by doing both first binding to FGFR and serving as a soluble FGFR coreceptor or first binding to FGF23 and serving as a circulating FGF23 binding partner. Either way, sKL mediates FGF23-FGFR binding in an FGFR isoform-specific manner, with highest FGF23-sKL affinities for FGFR1c and FGFR4, lower affinity for FGFR3c, and no binding to FGFR2c. Furthermore, in the absence of sKL, FGF23 can bind FGFR4 with low affinity, supporting the FGFR4-mediated actions of FGF23 elevations that have been reported in cells lacking klotho. 15-16 Example 2

To determine whether sKL interferes with the direct effects of FGF23 on cardiac myocytes, isolated NRVMs were treated with FGF23 for 48 hours, which induced hypertrophic growth as determined by an increase in the area of immunolabeled cells (FIG. 6A), as shown before.^{13,16,17} When NRVMs were cotreated with sKL, FGF23 did not induce hypertrophy, similar to the inhibitory actions observed in the presence of anti-FGF23 blocking antibody. Treatment with sKL by itself had no effect on the NRVM area. This finding is in line with previous studies showing that FGF23 treatment of isolated adult ventricular cardiac myocytes causes calcium mishandling, alters contractility, and triggers proarrhythmic calcium release, which can be blocked by SKL.⁶⁹ Furthermore, it was found that administration of sKL protects a mouse model of CKD from developing ventricular arrhythmias.⁷⁹ Combined, these studies indicate that sKL can directly inhibit the pathologic actions of FGF23 on cardiac myocytes, suggesting the potential for sKL administration as a novel cardioprotective therapy that might be effective in scenarios of systemic FGF23 elevations, such as CKD. However, attempts from various groups to purify sKL protein in larger amounts have failed, and sKL protein appears to be highly unstable.

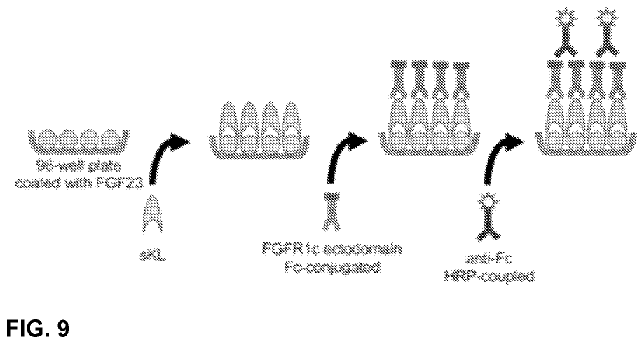

The structural analysis of the klotho complex revealed the presence of a Zn²⁺ ion located in the linker region between the extracellular KL1 and KL2 domains, which is crucial for stabilizing klotho's elongated structure, for forming the FGF23 binding pocket, and for FGF23-induced Ras/MAPK signaling.⁶⁵ A novel procedure was developed to synthesize and purify recombinant mouse and human sKL protein from stably transfected HEK293 cells in the absence of chelating agents, such as ethylenediamine tetraacetic acid (EDTA) and avoid nonphysiological elution conditions (Supplementary FIG. 8A). Because the function of sKL has been unknown, an assay to test the bioactivity of sKL is currently unavailable. On the basis of the finding that sKL can bind the ectodomain of different FGFR isoforms, with high binding affinity for FGFR1c, an assay was developed that can detect biologically active sKL by its ability to sequentially bind FGF23 and FGFR1c (FIG. 9). It was found that the assay can detect self-made sKL protein dissolved in physiologic buffer in a concentration-dependent manner, with a limit for linear detection in the range of 300 μg/ml (FIG. 6B). The assay was used to test the bioactivity of 2 commercially available sKL variants that have been used in various functional in vitro and in vivo studies.^{54,78} The first variant contained the ectodomain of klotho and showed significantly reduced binding as compared with the sKL protein (FIG. 6C), suggesting lower bioactivity. Because commercial sKL is stored in EDTA-based buffer, it is likely that the Zn²⁺ ion has been depleted, leading to structural changes and reduced activity of sKL. The second variant contained only the KL1 domain of klotho. The plate-based binding assay revealed that KL1 cannot mediate FGF23-FGFR1c binding and therefore lacks biological activity, at least activity that is mediated by FGF23 and FGFRs (FIG. 6C). This is in line with the recent structural study showing that sKL binding to FGF23 and FGFR1c is mediated by KL2 and the linker region connecting KL2 with KL1 but not by KL1.⁶⁵

The assay also detected sKL in protein extracts derived from HEK293 cells overexpressing sKL (FIG. 6D). Extracts from HEK293 cells overexpressing full-length klotho were negative in the detection assay, most likely on the basis of the unspecific accumulation of the transmembrane protein and thereby loss of bioactivity. Furthermore, the assay did not detect the transmembrane or soluble forms of β-klotho, indicating the specificity of the FGF23-FGFR detection assay for sKL (FIG. 6D).

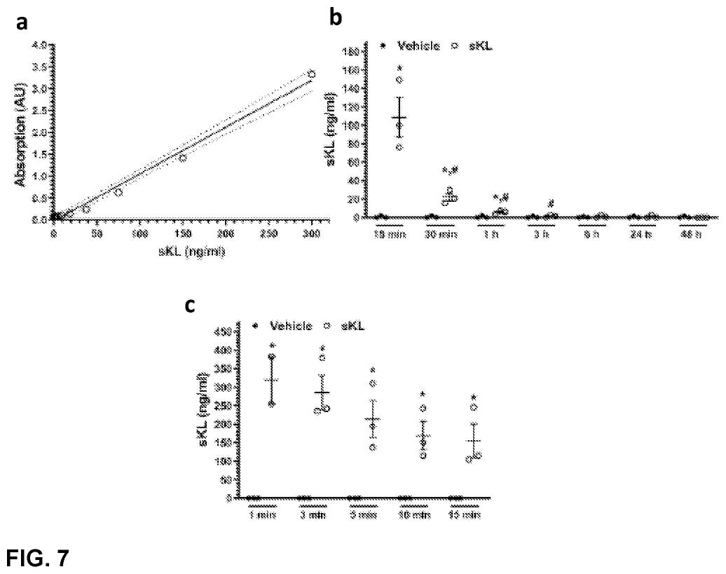

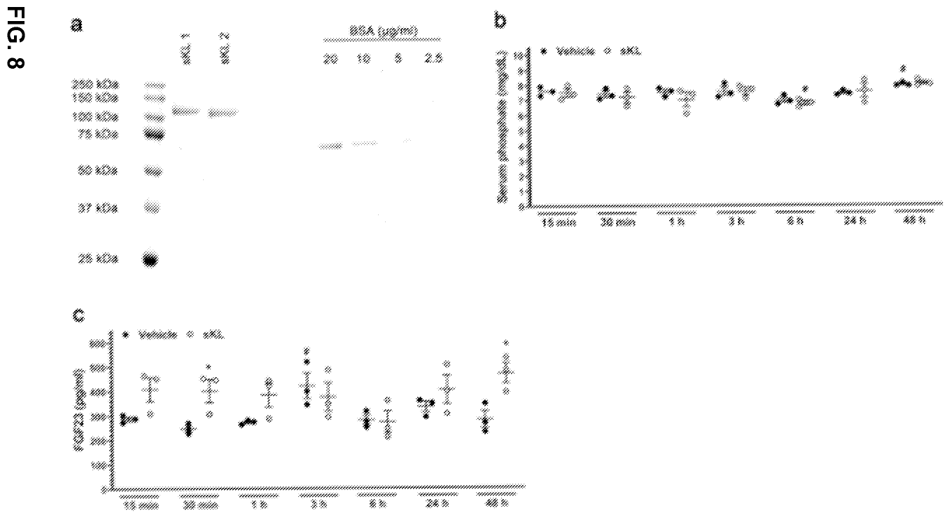

A reliable assay to detect sKL is needed to fully assess the potential of sKL as a clinical biomarker. To test whether the FGF23-FGFR1c binding assay could detect sKL in serum, a self-made murine sKL protein was administered to rats by i.v. injection and sKL was detected down to a concentration of 10 ng/ml (FIG. 7A). Serial bleeds post-injection over time revealed a half-life in the circulation of approximately 10 minutes for the recombinant sKL protein (FIG. 7B and FIG. 7C). Elevations in circulating sKL levels were accompanied by increases in serum concentrations of FGF23 while phosphate levels were not altered (FIG. 8B and FIG. 8C). Combined, the procedure to detect sKL provides a novel assay that specifically detects sKL in its bioactive form, defined by its ability to increase the binding affinity of FGF23 for FGFR1c, in cell extracts and in rodent blood. Furthermore, by modifying the sKL synthesis procedure, recombinant sKL protein was produced with high bioactivity that could be used for preclinical studies in animal models.

### Example 3

On the basis of the presence of HS in the FGF23/FGFR1c/sKL complex, it was decided to determine whether HS can mediate FGF23/FGFR binding in the absence of sKL and thereby serve as an independent cofactor for FGF23/FGFR binding as established for FGFR binding of paracrine FGFs. Heparin, a linear glycosaminoglycan structurally similar to HS, was used but with a higher degree of sulfation, that is used as an experimental proxy for HS. Fc-coupled FGFR ectodomains were immobilized on protein A/G beads, followed by coincubation with heparin and FGF23. It was found that FGFR4 efficiently coprecipitates FGF23, and weaker FGF23 binding to FGFR1c, FGFR2c, and FGFR3c was detected (FIG. 10A). In contrast, no FGF23 binding was detected to any of the FGFR isoforms in the absence of heparin. FGFR4 showed the strongest FGF23 binding in the presence of heparin, which was still below the binding affinity that was detected when sKL was used instead of heparin. Next, plates were coated with FGF23, followed by the sequential incubation with heparin, Fc-coupled FGFR ectodomains, HRP-coupled anti-Fc antibody, and HRP substrate. In the presence of heparin, FGF23 bound to FGFR4 and to a much lesser extent to FGFR2c (FIG. 10B), which occurred in a dose-dependent manner (FIG. 10C). As observed before, FGF23 bound FGFR4 in the absence of heparin in a dose-dependent manner and the affinity of FGF23 for FGFR4 was significantly increased by heparin (FIG. 10C). No binding between FGF23 and FGFR1c or FGFR3c was detected in the presence or absence of heparin (FIG. 10C). Overall, the study suggests that in the absence of klotho, heparin can mediate the interaction between FGF23 and FGFRs. In contrast to FGF23, the co-immunoprecipitation and plate-based binding studies showed that heparin does not mediate binding of FGF19 or FGF21 to any of the FGFR isoforms (FIG. 2C, FIG. 2D, and FIG. 3).

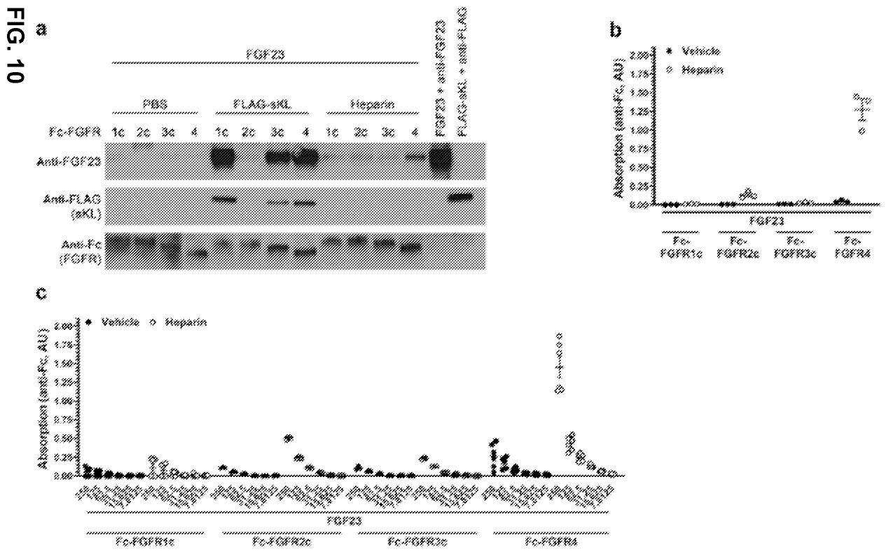

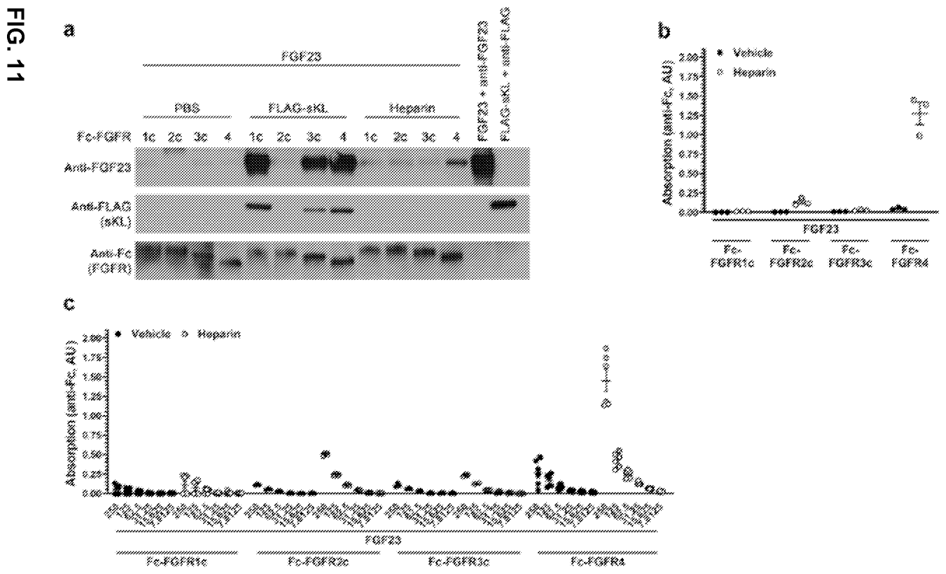

Surface plasmon resonance analyses were performed with heparin immobilized on a chip and purified soluble proteins to study which component of the FGF23/FGFR4/klotho complex could be directly bound by heparin and to determine precise binding affinities (FIG. 11A-C; Table A3 below). In Table A3, data with (+) in parentheses are the standard deviations (SD) from global fitting of five injections.

TABLE A3
Summary of Kinetic Data of Heparin Binding to FGF23,
FGFR4 and Klotho and of the FGF23-FGFR4 Interaction

| Interaction | kₐ (1/MS) | k_{d} (1/S) | K_{D} (M) |
|---|---|---|---|
| Heparin-PGF23 | 4.8 × 10⁵ | 3.6 × 10⁻³ | 7.6 × 10⁻⁹ |
| | (±3.5 × 10³) | (±3.3 × 10⁻ [symbol] ) | |
| Heparin-FGFR4 | 1.2 × 10⁶ | 3.3 × 10⁻ [symbol] | 2.8 × 10⁻⁹ |
| | (±8.1 × 10³) | (±2.3 × 10⁻⁵) | |
| Heparin-klotho | 8.0 × 10⁴ | 1.1 × 10⁻³ | 1.5 × 10⁻⁸ |
| | (±76) | (±4.3 × 10⁻⁶) | |
| PGF23-FGFR4 | 1.2 × 10³ | 0.04 | 3.2 × 10⁻ [symbol] |
| | (±2.5 × 10 [symbol] ) | (±4.1 × 10⁻⁴) | |

[symbol]

indicates data missing or illegible when filed

As expected,^{83,84} it was detected that heparin could bind FGFR4 with high affinity (dissociation constant KD=2.8 nM). Surprisingly, it was found that heparin can also bind FGF23 and klotho with similar affinities (KD=7.6 nM and KD=15 nM, respectively). These findings suggest that heparin can independently bind FGFR4, klotho, and FGF23 with high affinity. Next, the direct interaction between the immobilized ectodomain of FGFR4 and FGF23 was studied (FIG. 11D; Table A3) and low affinity binding (KD=320 nM) was detected, similar to other surface plasmon resonance studies⁸⁵ and in accordance with the low affinity binding detected by the plate-based assay (FIG. 5B and FIG. 10C). When the FGF23/FGFR4 binding study was conducted in the presence of heparin, an increase in binding affinity was detected by approximately 3-fold (FIG. 11E). When the study was repeated with sKL instead of heparin, FGF23/FGFR4 binding affinity increased by approximately 13-fold (FIG. 11F). Combined, these findings indicate that heparin and sKL both can act as independent coreceptors that mediate FGF23/FGFR4 binding, whereby sKL's effect on increasing FGFR4 affinity for FGF23 is approximately 4- to 5-fold higher than that of heparin.

### Example 4

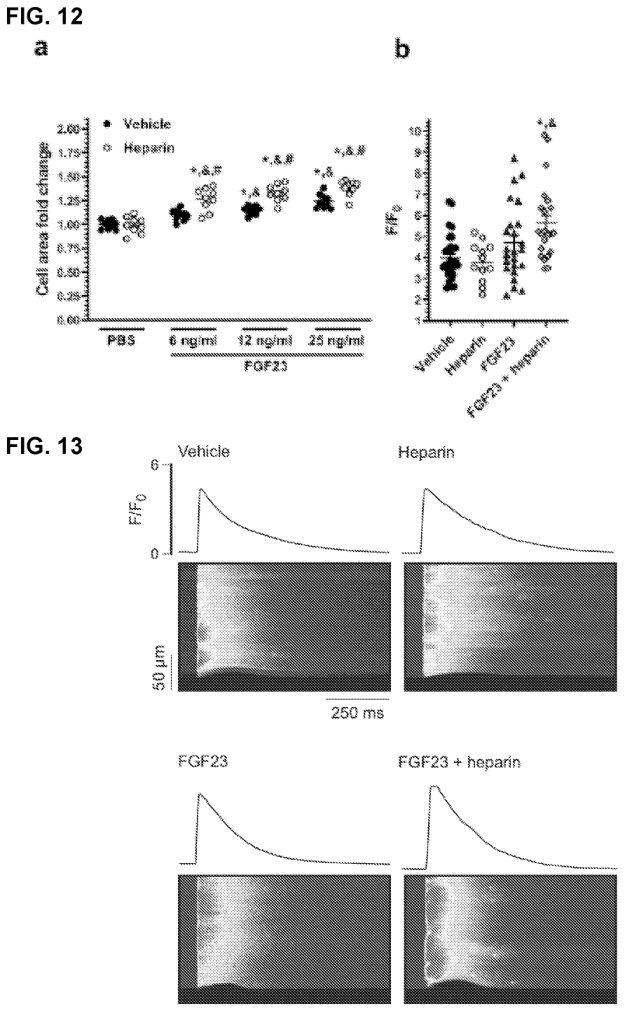

Because it was found that heparin mainly facilitates FGF23 binding to FGFR4, and it is known that the prohypertrophic actions of FGF23 on cardiac myocytes are mediated by FGFR4^{16,17,73} it was decided to determine whether heparin aggravates the cardiac effects of FGF23. First, NRVMs were cotreated with increasing concentrations of FGF23 and with heparin for 48 hours. It was found that the gradual FGF23-induced increase in the cell area was further elevated in the presence of heparin (FIG. 12A). Treatment with heparin by itself had no effect on the NRVM area. Because FGF23 alters calcium handling and contractility in isolated ARVMs, the potential effects of heparin was studied in this context. Perfusion of ARVMs with FGF23 for 2 minutes increased systolic [Ca²⁺]; transients, and this effect became statistically significant when cells were coperfused with FGF23 and heparin (FIG. 12B through FIG. 13).

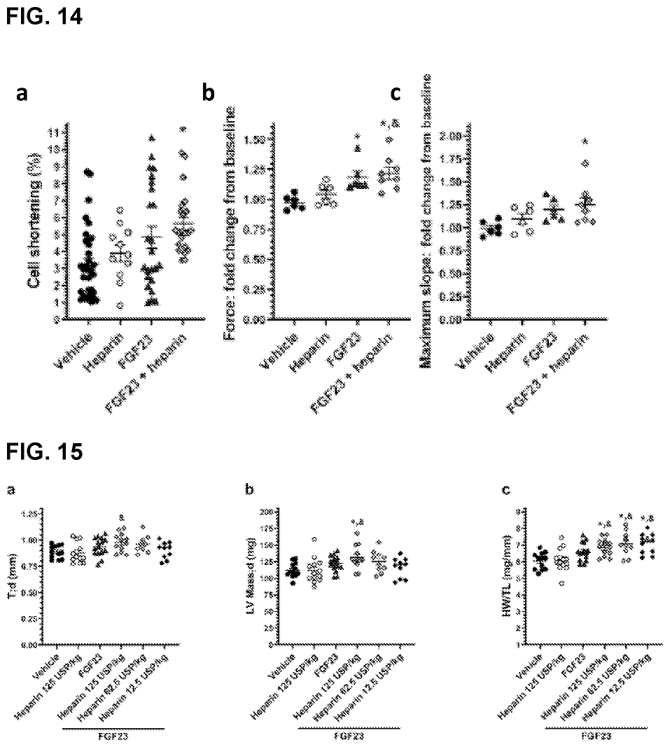

Furthermore, FGF23 increased cell contraction of ARVMs, which was significantly increased when cells were coperfused with FGF23 and heparin (FIG. 14A). In both ARVM studies, heparin by itself had no significant effects. It has been previously shown that the treatment of isolated adult mouse hearts with FGF23 for 30 minutes results in an increase in ventricular contraction as well as intracellular calcium.¹⁴ This acute FGF23 effect occurs in a concentration-dependent manner and is mediated by FGFR4.¹⁷ To determine whether heparin modulates FGF23's actions on cardiac contractility, the contractile responses were analyzed of isolated hearts from mice in the presence of FGF23 and heparin. Treatment with FGF23 increased cardiac contractile force compared with vehicle as previously reported.^{14,17,71} When FGF23 was coincubated with heparin before addition to the organ bath, a further increase in cardiac contractility force was detected (FIG. 14B and FIG. 14C). Combined, these findings suggest that heparin promotes the acute effects of FGF23 on the heart and further increases contractility properties and handling of intracellular calcium in cardiac myocytes and thereby might enhance calcium-dependent prohypertrophic signaling and calcium overload and trigger cardiac arrhythmogenicity.

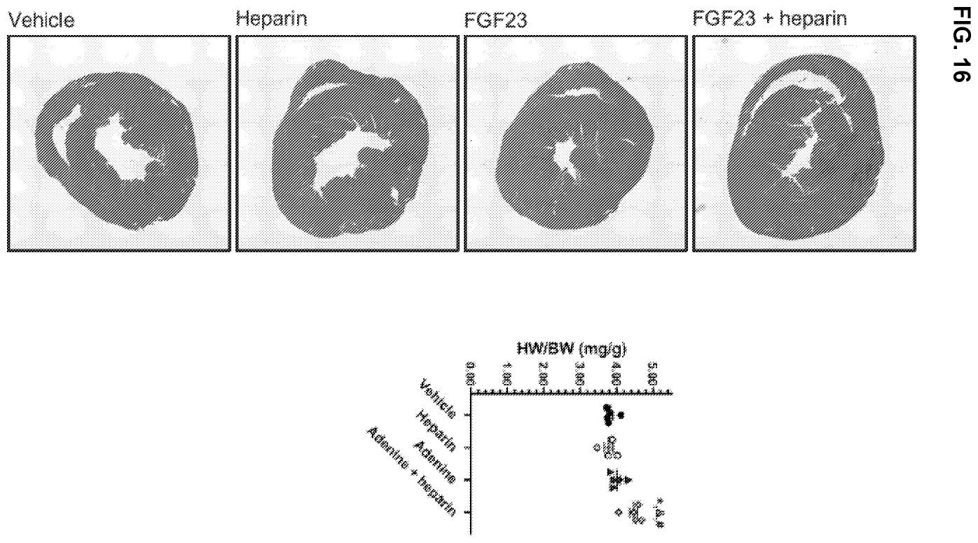

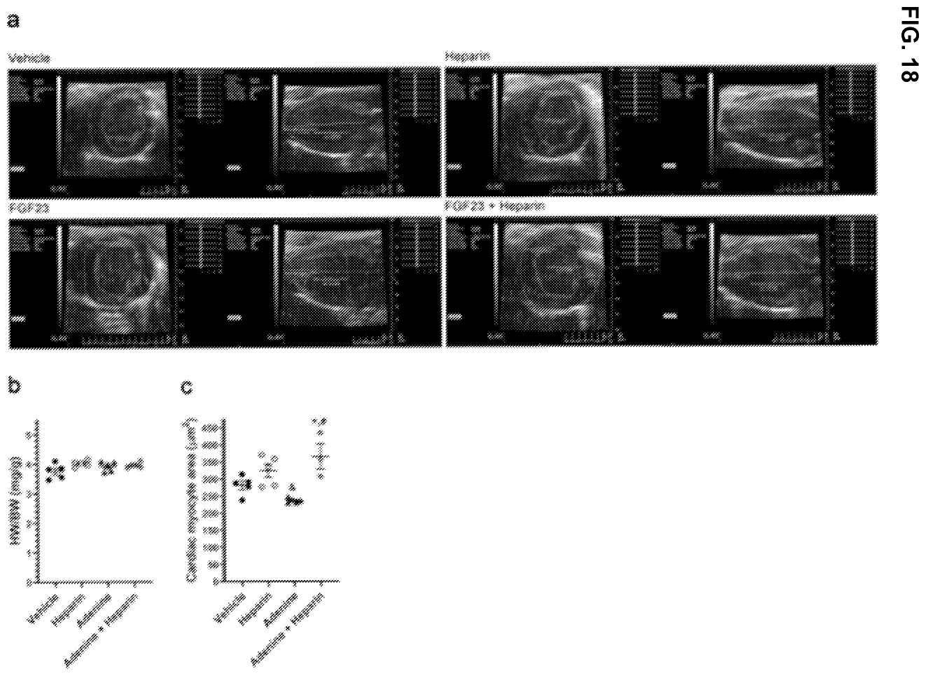

Finally, it was decided to determine whether heparin aggravates the cardiac effects of FGF23 in vivo. First, FGF23 and heparin were coinjected in mice. It has been shown before that 2 tail vein injections of recombinant FGF23 protein for 5 consecutive days induces cardiac hypertrophy, 13 which has been confirmed by others.^{72,86,87} This effect requires the presence of FGFR4 in cardiac myocytes and occurs independently of blood pressure elevations, suggesting that this is a mouse model of cardiac hypertrophy caused by the direct FGFR4-mediated actions of circulating FGF23 on the heart. Following the same protocol, male BALB/c mice were injected with 40 μg/kg of FGF23 and 125 USP/kg of heparin separately and together. Compared with vehicle and FGF23 injections, mice receiving FGF23 and heparin combined developed a significant increase in left ventricular wall thickness and left ventricular mass, as revealed by echocardiography (FIG. 15A, FIG. 15B, FIG. 18A, and Table A4, below), resulting in an increase in the ratio of heart weight to tibia length (FIG. 15C) and histological changes (FIG. 16A). In Table A4, values are expressed as mean±SEM. Comparison between groups was performed in form of a one-way ANOVA followed by a post-hoc Tukey test. A level of P<0.05 was accepted as statistically significant, with *p≤0.05 vs. vehicle,^{&}p≤0.05 vs. heparin 125 USP/kg, #p≤ 0.05 vs. FGF23, n=9-15.

TABLE A4
Ultrasound Parameters of Mice Receiving Serial FGF23 Injections

| | | | | FGF23 + | FGF23 + | FGF23 + |
|---|---|---|---|---|---|---|
| | | Heparin | | Heparin | Heparin | Heparin |
| | Vehicle | 125 USP/kg | FGF23 | 125 USP/kg | 62.5 USP/kg | 12.5 USP/kg |
| Endocardial | 10.17 ± 0.57 | 10.20 ± 0.0.50 | 9.93 ± 0.39 | 10.45 ± 0.26 | 10.23 ± 0.41 | 10.10 ± 0.30 |
| Area; d | | | | | | |
| (mm³) | | | | | | |
| Endocardial | 4.68 ± 0.36 | 5.47 ± 0.44 | 4.88 ± 0.34 | 5.24 ± 0.31 | 5.37 ± 0.34 | 5.45 ± 0.37 |
| Area; s | | | | | | |
| (mm³) | | | | | | |
| Epicardial | 22.60 ± 0.67 | 22.49 ± 0.78 | 23.07 ± 0.45 | 24.67 ± 0.49 | 23.86 ± 0.64 | 22.86 ± 0.63 |
| Area; d | | | | | | |
| (mm³) | | | | | | |
| Epicardial | 17.97 ± 0.65 | 18.22 ± 0.63 | 19.44 ± 0.46 | 20.65 ± 0.39** | 19.48 ± 0.74 | 19.57 ± 0.62 |
| Area: s | | | | | | |
| (mm³) | | | | | | |
| Endocardial | 54.15 ± 1.94 | 47.34 ± 2.41 | 51.38 ± 2.02 | 49.92 ± 2.53 | 47.70 ± 1.96 | 46.20 ± 3.02 |
| % FAC (%) | | | | | | |
| Endocardial | 66.17 ± 3.63 | 56.51 ± 4.08 | 61.57 ± 3.48 | 60.29 ± 3.85 | 53.82 ± 1.81 | 52.46 ± 2.575 |
| % EF (%) | | | | | | |
| Endocardial | 7.61 ± 0.14 | 7.23 ± 0.11 | 7.94 ± 0.09 | 7.70 ± 0.12 | 7.78 ± 0.09 | 7.64 ± 0.06 |
| major; d | | | | | | |
| (mm) | | | | | | |
| T; d (mm) | 0.89 ± 0.02 | 0.88 ± 0.03 | 0.94 ± 0.02 | 0.98 ± 0.03* | 0.96 ± 0.03 | 0.90 ± 0.02 |
| LV Mass; d | 112.20 ± 3.17 | 111.90 ± 5.23 | 122.10 ± 3.05 | 131.40 ± 5.21** | 125.40 ± 5.05 | 116.30 ± 4.74 |
| (mg) | | | | | | |
| Heart rate | 447.90 ± 4.67 | 448.40 ± 4.52 | 452.70 ± 4.48 | 453.70 ± 2.98 | 458.70 ± 4.56 | 454.20 ± 3.43 |
| (BPM) | | | | | | |

The increase in cardiac mass was not accompanied by changes in cardiac function, such as ejection fraction or fractional shortening (Table A4), similar to what was previously found in a genetic mouse model with systemic FGF23 elevations.⁷⁰ When the heparin concentration coinjected with FGF23 was reduced by 2- or 10-fold, changes in echocardiographic parameters were not significant (FIG. 15A and FIG. 15B), indicating a dose-dependent effect of heparin on the cardiac actions of FGF23. Second, it has been shown that the induction of kidney injury in wild-type mice by administration of an adenine-containing diet significantly increases serum FGF23 levels⁷⁴ and causes cardiac hypertrophy.⁷⁵ Kidney and cardiac injury are more severe in male mice.⁷⁶ Male and female BALB/c mice were placed on a 0.2% adenine diet for 10 weeks starting at 6 weeks of age. Throughout the feeding study, mice were injected via the tail vein with heparin at 125 USP/kg or saline 3 times per week. After 10 weeks, serum levels of FGF23 and creatinine were significantly elevated in male adenine diet-fed mice when compared with mice on control chow, and heparin injections had no additional effect on these parameters.

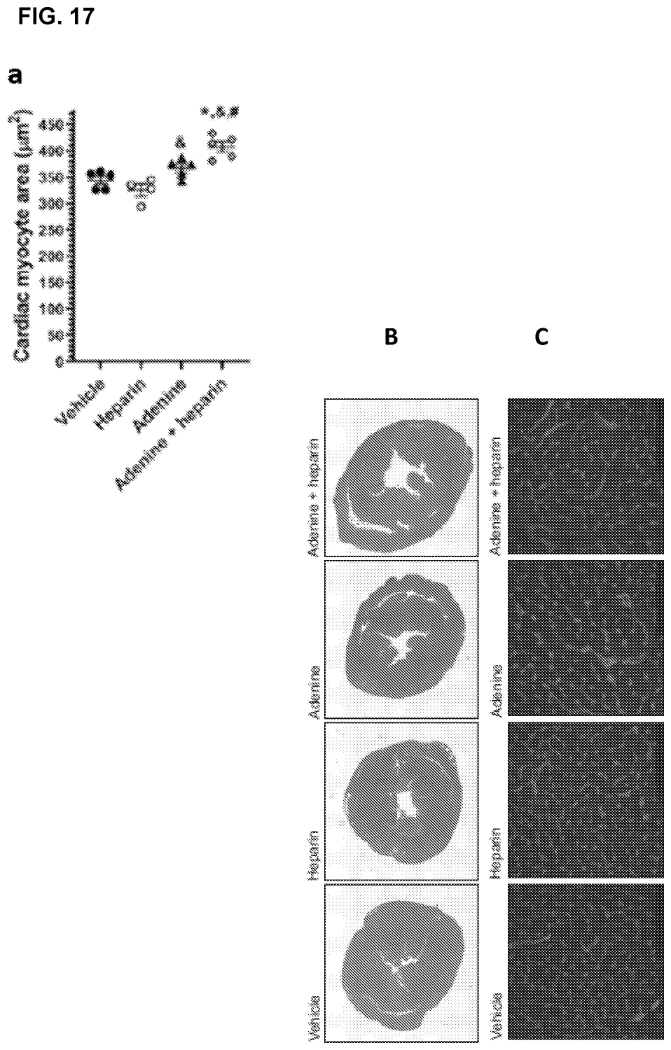

These findings indicate that in the adenine diet-fed mouse model, frequent heparin injections do not further elevate circulating FGF23 concentrations or worsen kidney injury. However, compared with mice receiving control chow, adenine mice showed increases in the ratio of heart weight to body weight as well as cross-sectional area of individual myocytes, which both were exacerbated by heparin injections (FIG. 16B, FIG. 17A through FIG. 17C). Heparin injections in mice on a normal diet had no effect on the heart. Furthermore, although female mice on an adenine diet did not develop kidney injury or cardiac hypertrophy (FIG. 18B), they had significantly elevated serum levels of FGF23 and phosphate. Heparin injections in female adenine mice induced a significant increase in the area of individual cardiac myocytes (FIG. 18C). Combined, the 2 animal models indicate that although frequent heparin injections by themselves do not affect the heart, they promote cardiac hypertrophy in the presence of systemic FGF23 elevations, as the case in CKD.

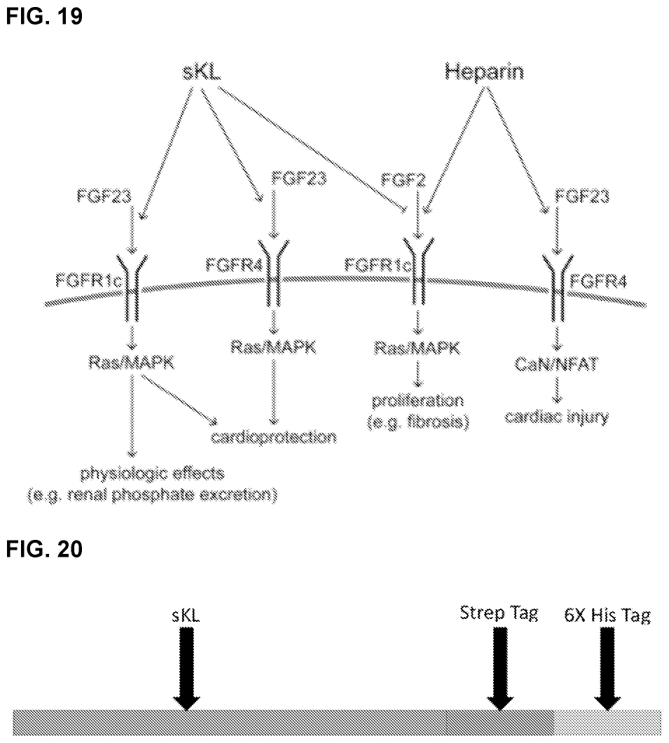

It was found that sKL and heparin act as FGFR coreceptors for FGF23 (FIG. 19). Both factors bind FGFRs independently from each other and in the absence of FGF23 and thereby increase the FGFR binding affinity for FGF23. sKL mediates FGF23 binding to various FGFR isoforms and favors FGFR1 in the absence of FGF23 and FGFR4 in the presence of FGF23. In contrast, heparin seems to mainly facilitate the FGF23 interaction with FGFR4. Heparin increases FGFR affinities of FGF23 to a lower extent than sKL. Furthermore, it was found that sKL and heparin can independently bind FGF23 in the absence of FGFRs and then mediate binding to FGFRs following the same FGFR isoform specificity as detected for the FGFR binding of sKL and heparin that occurs in the absence of FGF23. By doing so, sKL serves as a circulating FGF23 binding partner. However, on the basis of differences in binding affinities, sKL seems to favor the interaction with FGFR1 over FGF23. These findings also suggest that as known for paracrine FGFs, heparin promotes FGF23 binding to FGFRs in a klotho-independent manner and plays a dual role in the FGFR activation process. Heparin supports the formation of a stable FGF/FGFR/heparin ternary complex as well as FGFR/FGFR dimerization and subsequent activation of signaling events. Although compared to paracrine FGFs the affinity of FGF23 for heparin is lower, heparin can still act as a cofactor for FGF23-FGFR binding and might do so in an FGFR isoform-specific manner. Different from their effects on FGF23, heparin and sKL do not mediate binding of FGF19 or FGF21 to any of the FGFR isoforms, suggesting that not only sKL but also heparin acts as an FGF23-specific FGFR coreceptor.

The finding that heparin can bind FGF23 goes against the common conception that as an endocrine FGF, FGF23 underwent changes in the topology of its heparin-binding site during evolution, resulting in a loss of heparin-binding capability.⁴¹ Heparin binding of FGF23 has been previously detected in a nonquantitative manner and with low affinity using heparin columns.⁴⁰ Furthermore, heparin columns have been used to purify recombinant FGF23 protein.⁶⁵ Herein is provided the first quantitative data indicating that the interaction between FGF23 and heparin occurs with high affinity. This is in line with cell-based studies showing that cotreatment with heparin can increase the cellular effects of FGF23.^{80,85,88,89} The finding that sKL can bind heparin is surprising, because klotho does not seem to be in direct contact with heparin within the FGF23/FGFR1/klotho complex.⁶⁵ The biological relevance of the sKL-heparin interaction remains to be established. For both sKL and FGF23, the question arises how in the light of high-affinity binding to heparin these proteins can act as endocrine factors.

It is proposed that the novel mechanistic findings have several important implications for pathologies that are associated with elevated FGF23 levels and reduced klotho expression, such as CKD and associated cardiovascular injury. In the absence of klotho, FGF23 can bind FGFR4 with low affinity and thereby contribute to cardiac hypertrophy. It could be confirmed that the klotho-independent low-affinity binding between recombinant FGF23 and FGFR4 proteins, which was detectable only by the sensitive plate-based binding assay herein but not in less sensitive co-immunoprecipitation studies. It was found that sKL can bind FGFR4 and thereby increase FGF23 affinity and directly inhibit the pathologic actions of FGF23 on cardiac myocytes. Although the precise mechanism remains unknown, it can be postulated that sKL might induce a switch from FGF23-induced klotho-independent PLCγ/calcineurin/NFAT to klotho-dependent Ras/MAPK signaling and thereby from FGF23-induced pathologic to protective cardiac events. Alternatively, because sKL has a higher affinity for FGFR1 than for FGFR4, sKL might force FGF23 into FGFR1/sKL binding and signaling and away from klotho-independent prohypertrophic calcineurin/NFAT signaling that is mediated by FGFR4. The findings set the stage for preclinical CKD studies with the goal to test potential cardioprotective effects of sKL. Administration of sKL could have additional beneficial effects by increasing FGF23 responsiveness in physiologic target organs that, because of injury, have lost klotho expression, such as the kidney in CKD. It is possible that injected sKL binds to remaining FGFR1 molecules on proximal tubular epithelial cells, thereby generating high-affinity FGF23 binding sites, resulting in increased kidney responsiveness to FGF23 and kidney phosphate excretion. The finding that sKL can block the klotho-independent binding of FGFRs to paracrine FGFs suggests that sKL's beneficial functions are not only based on its modulation of FGF23 signaling but also mediated by the inhibition of mitogenic FGFR1c signaling induced by paracrine FGFs, which potentially results in antifibrotic and/or antitumor effects. It is proposed that the pleiotropic actions of sKL might be based on its involvement in various FGF-FGFR binding and subsequent signaling events.

Finally, it was found that heparin specifically increases FGF23 binding to FGFR4, the FGFR isoform that mediates the pathologic actions of FGF23 on cardiac myocytes, 16.73 and thereby promotes the acute effects of FGF23 on increasing contractility, dysregulating intracellular calcium, and enhancing arrhythmogenicity as well as the prolonged FGF23 effects of inducing hypertrophic cell growth. Combined, these cellular alterations might result in accelerated pathologic cardiac remodeling, as supported by the 2 mouse models with systemic FGF23 elevations, where frequent heparin injections worsened the cardiac phenotype, while heparin injections by themselves in healthy control animals had no cardiac effects. Patients with end-stage renal disease on hemodialysis frequently receive heparin infusions to prevent blood clotting during the dialysis process. Because hemodialysis does not reduce serum FGF23 levels, 90 these patients are exposed to constant systemic elevations of both FGF23 and heparin. Whether heparin injections contribute to the high rates of cardiovascular events and mortality in patients on hemodialysis remains to be studied.

### Example 5

Stably transfected HEK293T cells expressing Strep/His-tagged sKL (FIG. 20) are grown in liquid culture format. Cells are lyzed in RIPA buffer (50 mM Tris-HCl PH7.5, 200 mM NaCl, 1% Triton, 0.25% DOC, EDTA-free protease inhibitor cocktail). Clarified extract is applied to a cobalt talon column (GE Healthcare Life Sciences) using an ATKA Start Protein Purification System (GE), and run and washed in running buffer (50 mM sodium phosphate, 300 mM NaCl). sKL is eluted in running buffer containing 100 mM imidazole. No EDTA is added to eluted samples to bind leached metal ions. sKL-containing eluates are diluted 1:7 in buffer XT (100 mM Tris, 150 mM NaCl, without EDTA) and applied to a Streptactin XT column (IBA Life Sciences) via the AKTA System. sKL is eluted using buffer XT containing 50 mM biotin without EDTA. (See, for example, the procedure described in www.iba-lifesciences.com/isotope/2/2-4998-000-Manual Strep-TactinXT-Purification.pdf). sKL-containing aliquots are diluted 1:10 in heparin buffer (10 mM sodium phosphate) and applied to a heparin column via the AKTA System. sKL is eluted using heparin buffer containing 500 mM NaCl. The bioactivity of sKL aliquots is tested via the new sKL detection assay, determining sKL's ability to serve as a scaffold for FGF23 and FGFR1 binding. Positive aliquots are pooled and flash frozen for storage at −80° C.

### Example 6

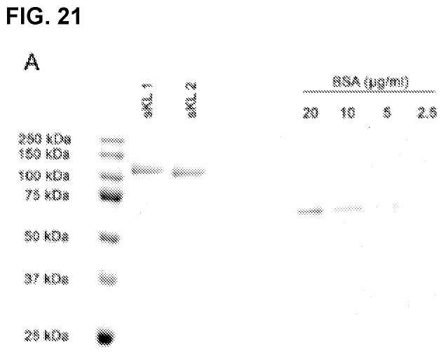

A novel procedure has been developed to synthesize and purify recombinant mouse and human sKL protein from stably transfected HEK293 cells in the absence of chelating agents, such as EDTA. FLAG- and Steptagged sKL is produced in Expi-HEK293 liquid cultures, and the secreted sKL protein is purified from the media using a 3-step affinity procedure (FIG. 21A).

An assay has been developed that can detect biologically active sKL by its ability to sequentially bind FGF23 and FGFR1c (FIG. 21B). The assay can detect a self-made sKL protein dissolved in a physiologic buffer (FIG. 22A). The bioactivity of two commercially available sKL variants that have been used for in vitro and in vivo studies was also tested. (See, for example, Hu, M. C. et al., Klotho and phosphate are modulators of pathologic uremic cardiac remodeling. JAm Soc Nephrol 26, 1290-1302, doi: 10.1681/ASN.2014050465 (2015); Hu, M. C. et al., Recombinant alpha-Klotho may be prophylactic and therapeutic for acute to chronic kidney disease progression and uremic cardiomyopathy. Kidney Int 91, 1104-1114, doi: 10.1016/j.kint.2016.10.034 (2017). The protein from R&D Systems contains the ectodomain of klotho and showed significantly reduced binding compared to the sKL protein (FIG. 22B). The protein from PeproTech contains only the KL1 domain and did not show any bioactivity in the assay. This is in line with a recent structural study showing that sKL binding to FGF23 and FGFR1c is mediated by KL2 and the KL1-KL2 linker region, but not by KL1. (See, for example, Chen, G. et al., alpha-Klotho is a non-enzymatic molecular scaffold for FGF23 hormone signalling. Nature 553, 461-466, doi: 10.1038/nature25451 (2018)). The assay also detected sKL in protein extracts derived from HEK293 cells overexpressing sKL (FIG. 22C). Extracts from HEK293 cells overexpressing full-length klotho were negative, most likely based on the unspecific accumulation of the transmembrane protein. Furthermore, the assay did not detect β-klotho, the FGFR co-receptor for FGF19 and FGF21, indicating the specificity of the FGF23-FGFR detection assay for sKL (FIG. 22C). To test whether the FGF23-FGFR1c binding assay could detect sKL in serum, the selfmade murine sKL protein was administered to rats by i.v. injection, and sKL was detected down to a concentration of 10 ng/ml (FIG. 22D). Serial bleedings post injection over time revealed a half-life in the circulation of under 15 minutes (FIG. 22E).

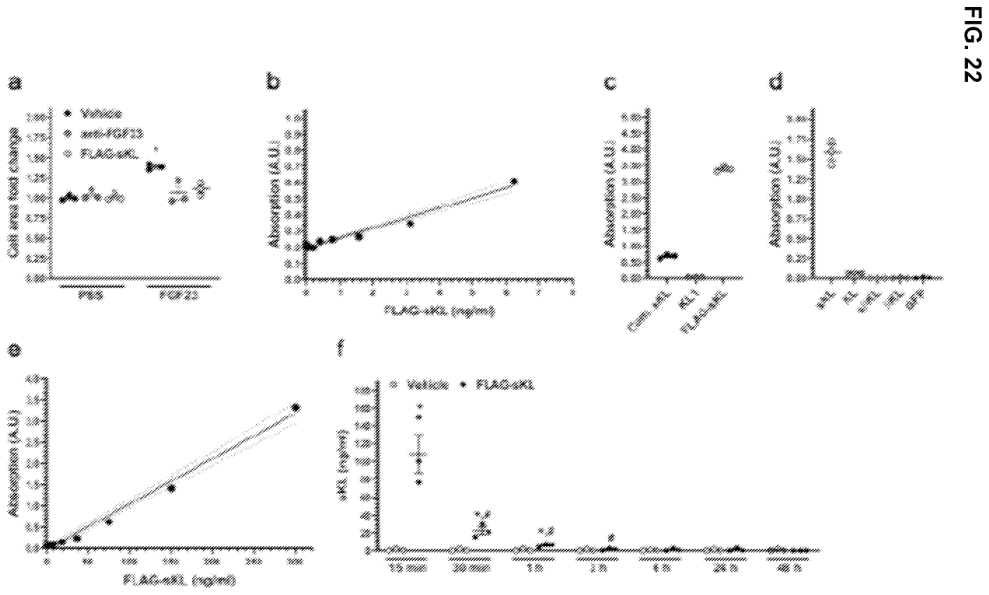

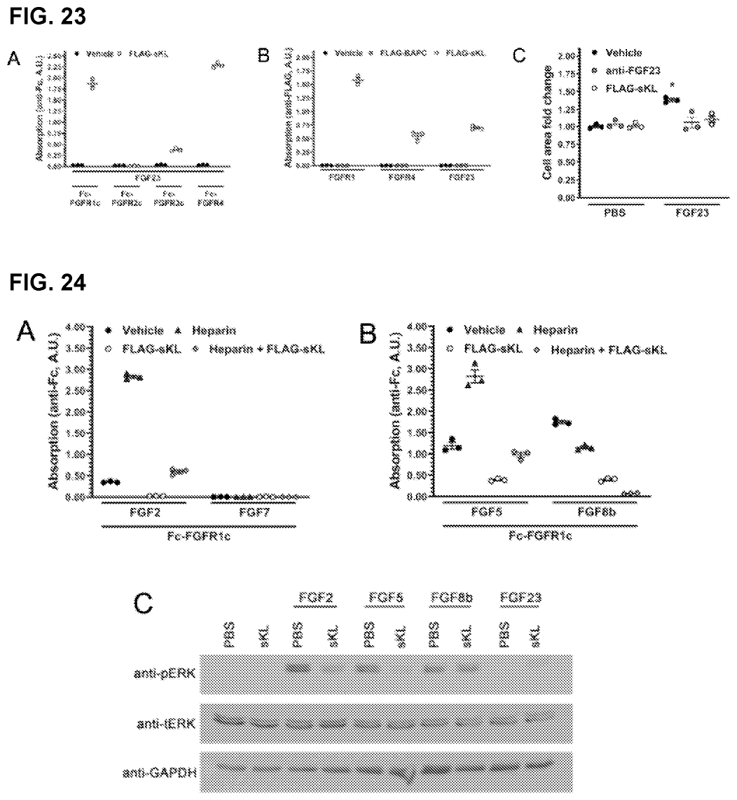

When the Fc-tagged FGFR isoforms that were added to the assay were varied, it was found that in the absence of sKL, FGF23 bound FGFR4 with low affinity but none of the other FGFR isoforms (FIG. 23A). By adding sKL, the affinities of FGFR1c and 4 for the complex were increased. It was then tested whether sKL can bind to FGF23 and/or the FGFRs separately. A modified version of the sKL activity assay was used, where wells are coated with either FGF23 or FGFRs. A FLAG-sKL purified from HEK293 cells was added, which was detected using anti-FLAG conjugated to HRP. It was found that sKL binds FGFR1 with the highest affinity, and FGF23 and FGFR4 to a lesser extent (FIG. 23B). The findings indicate that sKL increases FGFR binding affinity of FGF23 by both, first binding to FGFR and serving as a soluble FGFR co-receptor, or first binding to FGF23 serving as a circulating FGF23 binding partner. sKL mediates FGF23-FGFR binding in an FGFR isoforms-specific manner, with highest FGF23-sKL affinities for FGFR1c and FGFR4.

It was then determined whether sKL interferes with the established direct effects of FGF23/FGFR4 on cardiac myocytes. Isolated NRVMs were treated with FGF23 for 48 hours, which induced hypertrophic growth as determined by an increase in area of immunolabeled cells (FIG. 23C), as shown before. (See, for example, Grabner, A. et al., Activation of Cardiac Fibroblast Growth Factor Receptor 4 Causes Left Ventricular Hypertrophy. Cell Metab 22, 1020-1032, doi: 10.1016/j.cmet.2015.09.002 (2015); Faul, C. et al., FGF23 induces left ventricular hypertrophy. J Clin Invest 121, 4393-4408, doi: 10.1172/JC146122 (2011); and Grabner, A. et al FGF23/FGFR4-mediated left ventricular hypertrophy is reversible. Sci Rep 7, 1993, doi: 10.1038/s41598-017-02068-6 (2017). When NRVMs were co-treated with sKL, FGF23 did not induce hypertrophy, similar to the inhibitory actions of an anti-FGF23 blocking antibody. The finding is in line with a previous study showing that FGF23 treatment of isolated adult ventricular cardiac myocytes causes calcium mishandling and reduced contractility, which can be blocked by sKL. (See, for example, Navarro-Garcia, J. A. et al., Fibroblast growth factor-23 promotes rhythm alterations and contractile dysfunction in adult ventricular cardiomyocytes. Nephrol Dial Transplant, doi: 10.1093/ndt/gfy392 (2019)). Combined, both studies indicate that sKL can directly inhibit the pathologic actions of FGF23 on cardiac myocytes.

Wells were coated with FGF2 or FGF7, and sequentially incubated with Fc-FGFR1c that had been pre-incubated with sKL, followed by HRP-coupled anti-Fc, and HRP substrate. In the absence of SKL, FGFR1c bound FGF2, but not FGF7 (FIG. 24A), which is in line with studies showing that FGF7 interacts with b splice variants of FGFRs. (See, for example, Goetz, R. et al., Klotho coreceptors inhibit signaling by paracrine fibroblast growth factor 8 subfamily ligands. Mol Cell Biol 32, 1944-1954, doi: 10.1128/MCB.06603-11 (2012)). Adding heparin prior incubation with FGFR1c increased FGF2 binding to FGFR1c. In contrast, heparin did not induce binding between FGF7 and FGFR1c. In the presence of sKL, the binding of FGFR1c to FGF2 was reduced. Furthermore, sKL reduced the binding of FGFR1c to FGF5 and FGF8b (FIG. 24B). Next, HEK293 cells were co-incubated, which endogenously express various FGFR isoforms but lack klotho, with FGFs and sKL. In the absence of SKL, FGF2, FGF5, and FGF8b induced ERK1/2 phosphorylation, while endocrine FGF23 had no effect (FIG. 24C). Coincubation with sKL decreased phospho-ERK1/2 levels in cells treated with FGF2 and FGF5, but induced ERK1/2 phosphorylation when cells were incubated with FGF23.

### Example 7

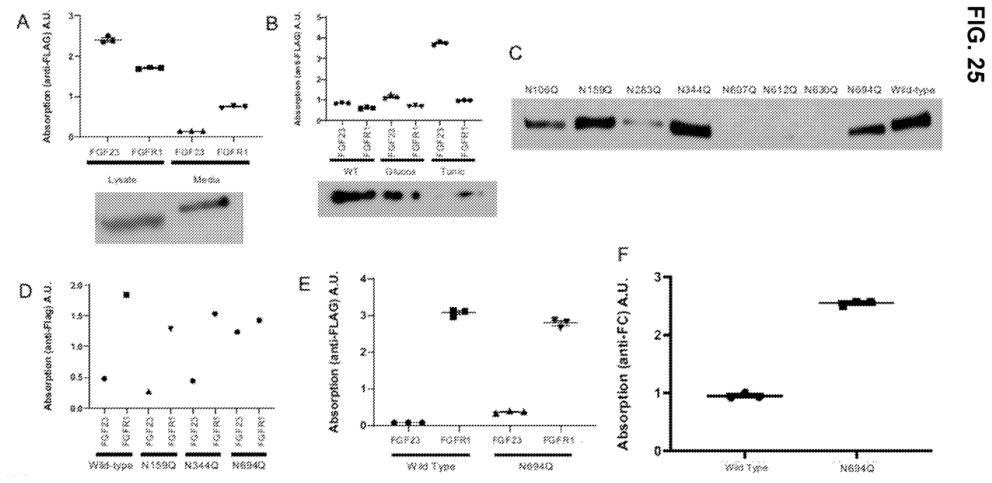

It was found that the FGF23 binding affinity of wildtype sKL is higher when the recombinant protein is derived from HEK293 lysates instead of supernatants (FIG. 25A). A smaller increase was detected for FGFR1c binding. Western blot analysis showed that the sKL protein in media has a higher molecular weight when compared to sKL from lysates, suggesting posttranslational modifications in secreted sKL (FIG. 25A). HEK293 cells were treated with two glycosylation inhibitors, glucosamine which prevents complex glycosylation, and tunicamycin which is an inhibitor of N-glycosylation. After 48 hours the media was tested in sKL binding assays. While tunicamycin treatment resulted in low expression levels of sKL, it was found that the small amount of sKL produced had much higher FGF23 binding affinity (FIG. 25B). Glucosmine also had an effect, suggesting that N-glycosylation inhibits FGF23 binding. Tunicamycin treatment did not affect the FGFR1c binding of sKL, suggesting that N-glycosylation of sKL does not affect FGFR1c binding. N-glycosylation occurs at a specific motif (Asn-X-Ser/Thr/Cys) where the Asn residue serves as the glycan acceptor site. The eight known N-glycosylation sites in sKL12 were mutated by substituting Asn with Gln. Given the structural similarities between Asn and Gln, this substitution removes glycosylation sites while maintaining structural integrity. Expression levels of the sKL mutants in HEK293 cells were determined, and strong expression of N159Q, N3440 and N6940 was detected (FIG. 25C). HEK293 cell media was then screened for FGF23 binding, and it was found that while N159Q and N3440 had similar affinity as wildtype sKL, variant N694Q showed increased FGF23 binding (FIG. 25D). Variant N6940 was further studied and the recombinant protein was purified from HEK293 cell supernatants. A 3-fold increase in FGF23 binding was detected when compared to the wildtype protein (FIG. 25E). These findings are in line with the recent crystal structure analysis of the FGF23/FGFR1/sKL complex, showing that Asn694 is at the interface of sKL's interaction with FGF23. (See, for example, Chen, G. et al., alpha-Klotho is a non-enzymatic molecular scaffold for FGF23 hormone signalling. Nature 553, 461-466, doi: 10.1038/nature25451 (2018)). It was studied whether this mutation affects the FGF23/FGFR1c/sKL complex. Similar to FGF23 binding alone, formation of the trimeric complex was increased by 3-fold when the sKL-N6940 was used instead of wildtype sKL (FIG. 25F). The findings indicate that by mutating Asn694, a sKL mimetic protein was generated with higher binding affinity for FGF23. While this mutation does not affect sKL's binding affinity for FGFR1c, it promotes FGF23/FGFR1c/sKL complex formation, which is known to mediate the physiologic actions of FGF23.

### REFERENCES

1. Quarles L D. Endocrine functions of bone in mineral metabolism regulation. J Clin Invest. 2008; 118:3820-3828.
2. Kuro-o M, Matsumura Y, Aizawa H, et al. Mutation of the mouse klotho gene leads to a syndrome resembling ageing. Nature. 1997; 390:45-51.
3. Urakawa I, Yamazaki Y, Shimada T, et al. Klotho converts canonical FGF receptor into a specific receptor for FGF23. Nature. 2006; 444:770-774.
4. Saito H, Maeda A, Ohtomo S, et al. Circulating FGF-23 is regulated by 1a, 25-dihydroxyvitamin D3 and phosphorus in vivo. J Biol Chem. 2005; 280:2543-2549.
5. Shimada T, Hasegawa H, Yamazaki Y, et al. FGF-23 is a potent regulator of vitamin D metabolism and phosphate homeostasis. J Bone Miner Res. 2004; 19:429-435.
6. Shimada T, Urakawa I, Yamazaki Y, et al. FGF-23 transgenic mice demonstrate hypophosphatemic rickets with reduced expression of sodium phosphate cotransporter type 11a. Biochem Biophys Res Commun. 2004; 314:409-414.
7. Saito H, Kusano K, Kinosaki M, et al. Human fibroblast growth factor-23 mutants suppress Na+-dependent phosphate co-transport activity and 10,25-dihydroxyvitamin D3 production. J Biol Chem. 2003; 278:2206-2211.
8. Isakova T, Gutierrez O M, Wolf M. A blueprint for randomized trials targeting phosphorus metabolism in chronic kidney disease. Kidney Int. 2009; 76:705-716.
9. Isakova T, Wahl P, Vargas G S, et al. Fibroblast growth factor 23 is elevated before parathyroid hormone and phosphate in chronic kidney disease. Kidney Int. 2011; 79:1370-1378.
10. Isakova T, Xie H, Yang W, et al. Fibroblast growth factor 23 and risks of mortality and end-stage renal disease in patients with chronic kidney disease. JAMA. 2011; 305:2432-2439.
11. Gutierrez O M, Mannstadt M, Isakova T, et al. Fibroblast growth factor 23 and mortality among patients undergoing hemodialysis. N Engl J Med. 2008; 359:584-592.
12. Richter B, Faul C. FGF23 actions on target tissues—with and without klotho. Front Endocrinol (Lausanne). 2018; 9:189.
13. Faul C, Amaral A P, Oskouei B, et al. FGF23 induces left ventricular hypertrophy. J Clin Invest. 2011; 121:4393-4408.
14. Touchberry C D, Green™, Tchikrizov V, et al. FGF23 is a novel regulator of intracellular calcium and cardiac contractility in addition to cardiac hypertrophy. Am J Physiol Endocrinol Metab. 2013; 304: E863-E873.
15. Singh S, Grabner A, Yanucil C, et al. Fibroblast growth factor 23 directly targets hepatocytes to promote inflammation in chronic kidney disease. Kidney Int. 2016; 90:985-996.
16. Grabner A, Amaral A P, Schramm K, et al. Activation of cardiac fibroblast growth factor receptor 4 causes left ventricular hypertrophy. Cell Metab. 2015; 22:1020-1032.
17. Grabner A, Schramm K, Silswal N, et al. FGF23/FGFR4-mediated left ventricular hypertrophy is reversible. Sci Rep. 2017; 7:1993.
18. Murphy D, McCulloch C E, Lin F, et al. Trends in prevalence of chronic kidney disease in the United States. Ann Intern Med. 2016; 165:473-481.
19. Tonelli M, Wiebe N, Culleton B, et al. Chronic kidney disease and mortality rislc a systematic review. J Am Soc Nephrol. 2006; 17:2034-2047.
20. Go A S, Chertow G M, Fan D, et al. Chronic kidney disease and the risks of death, cardiovascular events, and hospitalization. N Engl J Med. 2004; 351:1296-1305.
21. Zoccali C, Benedetto F A, Mallamaci F, et al. Prognostic value of echocardiographic indicators of left ventricular systolic function in asymptomatic dialysis patients. J Am Soc Nephrol. 2004; 15:1029-1037.
22. Foley R N, Parfrey P S, Harnett J D, et al. Clinical and echocardiographic disease in patients starting end-stage renal disease therapy. Kidney Int. 1995; 47:186-192.
23. London G M, Pannier B, Guerin A P, et al. Alterations of left ventricular hypertrophy in and survival of patients receiving hemodialysis: follow-up of an interventional study. J Am Soc Nephrol. 2001; 12:2759-2767.
24. Paoletti E, Bellino D, Cassottana P, et al. Left ventricular hypertrophy in nondiabetic predialysis CKD. Am J Kidney Dis. 2005; 46:320-327.
25. Appel U, Wright J T Jr, Greene T, et al. Intensive blood-pressure control in hypertensive chronic kidney disease. N Engl J Med. 2010; 363:918-929.
26. Baigent C, Landray M J, Reith C, et al. The effects of lowering LDL cholesterol with simvastatin plus ezetimibe in patients with chronic kidney disease (Study of Heart and Renal Protection): a randomised placebo-controlled trial. Lancet. 2011; 377:2181-2192.
27. Lowrie E G, Huang W H, Lew N L Death risk predictors among peritoneal dialysis and hemodialysis patients: a preliminary comparison. Am J Kidney Dis. 1995; 26:220-228.
28. Zager P G, Nikolic J, Brown R H, et al. Medical Directors of Dialysis Clinic, Inc. “U” curve association of blood pressure and mortality in hemodialysis patients. Kidney Int. 1998; 54:561-569.
29. Gross M L, Ritz E. Hypertrophy and fibrosis in the cardiomyopathy of uremia-beyond coronary heart disease. Semin Dial. 2008; 21:308-318.
30. Rambausek M, Ritz E, Mall G, et al. Myocardial hypertrophy in rats with renal insufficiency. Kidney Int. 1985; 28:775-782.
31. Siedlecki A M, Jin X, Muslin A J. Uremic cardiac hypertrophy is reversed by rapamycin but not by lowering of blood pressure. Kidney Int. 2009; 75:800-808.
32. Ritz E. Left ventricular hypertrophy in renal disease: beyond preload and afterload. Kidney Int. 2009; 75:771-773.
33. Ayus J C, Mizani M R, Achinger S G, et al. Effects of short daily versus conventional hemodialysis on left ventricular hypertrophy and inflammatory markers: a prospective, controlled study. J Am Soc Nephrol. 2005; 16:2778-2788.
34. Winchester J F, Audia P F. Extracorporeal strategies for the removal of middle molecules. Semin Dial. 2006; 19:110-114.
35. Itoh N, Ornitz D M. Evolution of the Fgf and Fgfr gene families. Trends Genet. 2004; 20:563-569.
36. Eswarakumar V P, Lax I, Schlessinger J. Cellular signaling by fibroblast growth factor receptors. Cytokine Growth Factor Rev. 2005; 16:139-149.
37. Schlessinger J, Plotnikov A N, Ibrahimi O A, et al. Crystal structure of a ternary FGF-FGFR-heparin complex reveals a dual role for heparin in FGFR binding and dimerization. Mol Cell. 2000; 6:743-750.
38. Ibrahimi O A, Yeh B K, Eliseenkova A V, et al. Analysis of mutations in fibroblast growth factor (FGF) and a pathogenic mutation in FGF receptor (FGFR) provides direct evidence for the symmetric two-end model for FGFR dimerization. Mol Cell Biol. 2005; 25:671-684.
39. Goetz R, Ohnishi M, Kir S, et al. Conversion of a paracrine fibroblast growth factor into an endocrine fibroblast growth factor. J Biol Chem. 2012; 287:29134-29146.
40. Asada M, Shinomiya M, Suzuki M, et al. Glycosaminoglycan affinity of the complete fibroblast growth factor family. Biochim Biophys Acta. 2009; 1790:40-48.
41. Goetz R, Beenken A, Ibrahimi O A, et al. Molecular insights into the klotho-dependent, endocrine mode of action of fibroblast growth factor 19 subfamily members. Mol Cell Biol 2007; 27:3417-3428.
42. Harmer N J, Pellegrini L, Chirgadze D, et al. The crystal structure of fibroblast growth factor (FGF) 19 reveals novel features of the FGF family and offers a structural basis for its unusual receptor affinity. Biochemistry. 2004; 43:629-640.
43. Kurosu H, Ogawa Y, Miyoshi M, et al. Regulation of fibroblast growth factor-23 signaling by klotho. J Biol Chem. 2006; 281:6120-6123.
44. Nakatani T, Ohnishi M, Razzaque M S. Inactivation of klotho function induces hyperphosphatemia even in presence of high serum fibroblast growth factor 23 levels in a genetically engineered hypophosphatemic (Hyp) mouse model. FASEB J. 2009; 23:3702-3711.
45. Chen C D, Podvin S, Gillespie E, et al. Insulin stimulates the cleavage and release of the extracellular domain of klotho by ADAM10 and ADAM17. Proc Natl Acad Sci USA. 2007; 104:19796-19801.
46. Chen C D, Tung T Y, Liang J, et al. Identification of cleavage sites leading to the shed form of the anti-aging protein klotho. Biochemistry. 2014; 53:5579-5587.
47. Lindberg K, Amin R, Moe O W, et al. The kidney is the principal organ mediating klotho effects. J Am Soc Nephrol. 2014; 25:2169-2175.
48. Hu M C, Shi M, Zhang J, et al. Renal production, uptake, and handling of circulating aKlotho. J Am Soc Nephrol. 2016; 27:79-90.
49. Imura A, Iwano A, Tohyama O, et al. Secreted Klotho protein in sera and CSF: implication for post-translational cleavage in release of Klotho protein from cell membrane. FEBS Lett. 2004; 565:143-147.
50. Kurosu H, Yamamoto M, Clark J D, et al. Suppression of aging in mice by the hormone Klotho. Science. 2005; 309:1829-1833.
51. Hu M C, Shiizaki K, Kuro-o M, Moe O W. Fibroblast growth factor 23 and Klotho: physiology and pathophysiology of an endocrine network of mineral metabolism. Annu Rev Physiol. 2013; 75:503-533.
52. Barker S L, Pastor J, Carranza D, et al. The demonstration of aKlotho deficiency in human chronic kidney disease with a novel synthetic antibody. Nephrol Dial Transplant. 2015; 30:223-233.
53. Sakan H, Nakatani K, Asai O, et al. Reduced renal α-Klotho expression in CKD patients and its effect on renal phosphate handling and vitamin D metabolism. PLOS One. 2014; 9: e86301.
54. Hu M C, Shi M, Cho Hi, et al. Klotho and phosphate are modulators of pathologic uremic cardiac remodeling. J Am Soc Nephrol. 2015; 26:1290-1302.
55. Hu M C, Shi M, Zhang J, et al. Klotho deficiency causes vascular calcification in chronic kidney disease. J Am Soc NephroL 2011; 22:124-136.
56. Yang K, Wang C, Nie L et al. Klotho protects against indoxyl sulphateinduced myocardial hypertrophy. J Am Soc Nephrol. 2015; 26:2434-2446.
57. Xie J, Cha S K, An S W, et al. Cardioprotection by Klotho through downregulation of TRPC6 channels in the mouse heart. Nat Commun. 2012; 3:1238.
58. Xie J, Yoon J, An S W, et al. Soluble klotho protects against uremic cardiomyopathy independently of fibroblast growth factor 23 and phosphate. J Am Soc NephroL 2015; 26:1150-1160.
59. Grabner A, Faul C. The role of fibroblast growth factor 23 and Klotho in uremic cardiomyopathy. Curr Opin Nephrol Hypertens. 2016; 25:314-324.
60. Hu M C, Shi M, Zhang J, et al. Klotho: a novel phosphaturic substance acting as an autocrine enzyme in the renal proximal tubule. FASEB J. 2010; 24:3438-3450.
61. Chang Q, Hoefs S, van der Kemp A W, et al. The (3-glucuronidase klotho hydrolyzes and activates the TRPV5 channel. Science. 2005; 310:490-493.
62. Cha S K, Ortega B, Kurosu H, et al. Removal of sialic acid involving Klotho causes cell-surface retention of TRPV5 channel via binding to galectin-1. Proc Natl Acad Sci USA. 2008; 105:9805-9810.
63. Dalton G, An SW, A1-Juboori S I, et al. Soluble klotho binds monosialoganglioside to regulate membrane microdomains and growth factor signaling. Proc Natl Acad Sci USA. 2017; 114:752-757.
64. Dalton G D, Xie J, An S W, Huang C L. New insights into the mechanism of action of soluble klotho. Front Endocrinol (Lausanne). 2017; 8:323.
65. Chen G, Liu Y, Goetz R, et al. α-Klotho is a non-enzymatic molecular scaffold for FGF23 hormone signalling. Nature. 2018; 553:461-466.
66. Ogawa Y, Kurosu H, Yamamoto M, et al. r3Klotho is required for metabolic activity of fibroblast growth factor 21. Proc Natl Acad Sci USA. 2007; 104:7432-7437.
67. Shi S Y, Lu Y W, Liu Z, et al. A biparatopic agonistic antibody that mimics fibroblast growth factor 21 ligand activity. J Biol Chem. 2018; 293:5909-5919.
68. Shalhoub V, Shatzen E M, Ward S C, et al. FGF23 neutralization improves chronic kidney disease-associated hyperparathyroidism yet increases mortality. J Clin Invest. 2012; 122:2543-2553.
69. Navarro-Garda J A, Delgado C, Fernandez-Velasco M, et al. Fibroblast growth factor-23 promotes rhythm alterations and contractile dysfunction in adult ventricular cardiomyocytes. Nephrol Dial Transplant. 2019; 34:1864-1875.
70. Wacker M J, Touchberry C D, Silswal N, et al. Skeletal muscle, but not cardiovascular function, is altered in a mouse model of autosomal recessive hypophosphatemic rickets. Front Physiol. 2016; 7:173.
71. Avin K G, Vallejo J A, Chen N X, et al. Fibroblast growth factor 23 does not directly influence skeletal muscle cell proliferation and differentiation or ex vivo muscle contractility. Am J Physiol Endocrinol Metab. 2018; 315: E594-E604.
72. Andrukhova O, Slavic S, Smorodchenko A, et al. FGF23 regulates renal sodium handling and blood pressure. EMBO Mol Med. 2014; 6:744-759.
73. Han X, Cai C, Xiao Z, Quarles L D. FGF23 induced left ventricular hypertrophy mediated by FGFR4 signaling in the myocardium is attenuated by soluble Klotho in mice. J Mol Cell Cardio!. 2019; 138:66-74.
74. Jia T, Olauson H, Lindberg K, et al. A novel model of adenine-induced tubulointerstitial nephropathy in mice. BMC NephroL 2013; 14:116.
75. Clinkenbeard E L, Noonan M L Thomas J C, et al. Increased FGF23 protects against detrimental cardio-renal consequences during elevated blood phosphate in CKD. JCI Insight. 2019; 4: e123817.
76. Diwan V, Small D, Kauter K, et al. Gender differences in adenine-induced chronic kidney disease and cardiovascular complications in rats. Am J Physiol Renal Physiol. 2014; 307: F1169-F1178.
77. Goetz R, Ohnishi M, Ding X, et al. Klotho coreceptors inhibit signaling by paracrine fibroblast growth factor 8 subfamily ligands. Mol Cell Biol 2012; 32:1944-1954.
78. Hu M C, Shi M, Gillings N, et al. Recombinant α-Klotho may be prophylactic and therapeutic for acute to chronic kidney disease progression and uremic cardiomyopathy. Kidney Int. 2017; 91:1104-1114.
79. Navarro-Garcia J A, Salguero-Bodes R, Gonzalez-Lafuente L, et al. The antiaging factor Klotho protects against acquired long QT syndrome induced by uremia and promoted by fibroblast growth factor 23. BMC Med. 2022; 20:14.
80. Xiao Z, King G, Mancarella S, et al. FGF23 expression is stimulated in transgenic α-Klotho longevity mouse model. JCI Insight. 2019; 4: e132820.
81. Wang Q Su W, Shen Z, Wang R. Correlation between soluble a-Klotho and renal function in patients with chronic kidney disease: a review and meta-analysis. Biomed Res Int. 2018; 2018:9481475.
82. Olauson H, Mencke R, Hillebrands J L, Larsson T E. Tissue expression and source of circulating aKlotho. Bone. 2017; 100:19-35.
83. Gao G, Goldfarb M. Heparin can activate a receptor tyrosine kinase. EMBO J. 1995; 14:2183-2190.
84. Loo B M, Kreuger J, Jallcanen M, et al. Binding of heparin/heparan sulfate to fibroblast growth factor receptor 4. J Biol Chem. 2001; 276:16868-16876.
85. Yu X, Ibrahimi O A, Goetz R, et al. Analysis of the biochemical mechanisms for the endocrine actions of fibroblast growth factor-23. Endocrinology. 2005; 146:4647-4656.
86. Slavic S, Ford K, Modert M, et al. Genetic ablation of Fgf23 or klotho does not modulate experimental heart hypertrophy induced by pressure overload. Sci Rep. 2017; 7:11298.
87. Han X, Rossi, Kolumam G, et al. Cardiovascular effects of renal distal tubule deletion of the FGF receptor 1 gene. JAm Soc NephroL 2018; 29:69-80.
88. Zhang X, Ibrahimi O A, Olsen S K, et al. Receptor specificity of the fibroblast growth factor family: the complete mammalian FGF family. J Biol Chem. 2006; 281:15694-15700.
89. Shalhoub V, Ward S C, Sun B, et al. Fibroblast growth factor 23 (FGF23) and α-klotho stimulate osteoblastic MC3T3.E1 cell proliferation and inhibit mineralization. Cakif Tissue Int. 2011; 89:140-150.
90. Wolf M. Forging forward with 10 burning questions on FGF23 in kidney disease. JAm Soc NephroL 2010; 21:1427-1435

### EXEMPLARY EMBODIMENTS

The following are non-limiting examples of specific embodiments of the subject matter disclosed above. This disclosure specifically But nonexclusively supports claims to these embodiments:

Emb. 1 A method of treating a disease associated with fibroblast growth factor 23 (FGF23) in a subject in need thereof comprising: administering a soluble α-klotho (sKL) compound to the subject in an amount effective to treat the disease.

Emb. 2 A method of preventing a disease associated with fibroblast growth factor 23 (FGF23) in a subject in need thereof comprising: administering a soluble α-klotho (SKL) compound to the subject in an amount effective to prevent the disease.

Emb. 3 A method of treating a disease associated with fibroblast growth factor 23 (FGF23) in a subject in need thereof comprising: increasing the expression of a soluble α-klotho (sKL) compound in the subject to an extent effective to treat the disease.

Emb. 4 A method of preventing a disease associated with fibroblast growth factor 23 (FGF23) in a subject in need thereof comprising: increasing the expression of a soluble α-klotho (sKL) compound in the subject to an extent effective to prevent the disease.

Emb. 5 A medicament for the treatment and/or prevention of a disease associated with fibroblast growth factor 23 (FGF23) in a subject comprising: a therapeutically effective amount of a soluble α-klotho (sKL) compound.

Emb. 6 A soluble α-klotho (sKL) compound for use in the treatment or prevention of a disease associated with fibroblast growth factor 23 (FGF23).

Emb. 7 A substance for treating or preventing a disease associated with fibroblast growth factor 23 (FGF23) comprising a soluble α-klotho (sKL) compound as a main ingredient.

Emb. 8 A use of a soluble α-klotho (sKL) compound for the manufacture of a medicament for the treatment of a disease associated with fibroblast growth factor 23 (FGF23).

Emb. 9 Any one of the embodiments above, wherein the disease associated with FGF23 is at least one of the following: kidney disease, chronic kidney disease, hyperphosphatemia, cardiovascular disease, vascular calcification, cardiovascular fibrosis, stress-induced cardiac injury, apoptosis, oxidative stress, cellular senescence, normal aging, tissue fibrosis, and inflammation.

Emb. 10 Any one of the embodiments above, wherein the sKL compound is administered in a dosage selected from about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 μg of the sKL compound per kg of the subject.

Emb. 11 Any one of the embodiments above, wherein the sKL compound is administered in a dosage selected from at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 μg of the sKL compound per kg of the subject.

Emb. 12 Any one of the embodiments above, wherein the sKL compound is administered in a dosage selected from at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 μg of the sKL compound per kg of the subject.

Emb. 13 Any one of embodiments 1-Emb. 2, Emb. 5, and Emb. 10-Emb. 12, wherein the sKL compound is administered at a frequency selected from more than once per day, once per day, once per week, once per month, and once per year.

Emb. 14 Any one of the embodiments above, wherein the sKL compound is administered in a dosage selected from about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 μg of the sKL compound per dosage.

Emb. 15 Any one of the embodiments above, wherein the sKL compound is administered in a dosage selected from at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 μg of the sKL compound per dosage.

Emb. 16 Any one of the embodiments above, wherein the sKL compound is administered in a dosage selected from at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 μg of the sKL compound per dosage.

Emb. 17 The medicament of any one of embodiments Emb. 5 and 8, further comprising a pharmaceutically acceptable base addition salt selected from sodium, potassium, calcium, ammonium, organic amino, magnesium salt, and combinations thereof.

Emb. 18 The medicament of any one of embodiments Emb. 5, 8 and Emb. 17, further comprising a pharmaceutically acceptable acid addition salt derived from an acid selected from hydrochloric, hydrobromic, nitric, carbonic, monohydrogencarbonic, phosphoric, monohydrogenphosphoric, dihydrogenphosphoric, sulfuric, monohydrogensulfuric, hydriodic, phosphorous acids, acetic, propionic, isobutylric, oxalic, maleic, malonic, benzoic, succinic, suberic, fumaric, mandelic, phthalic, benzensulfonic, p-tolylsulfonic, citric, tartaric, methanesulfonic, arginate, glucuronic, galactunoric, and combinations thereof.

Emb. 19 The medicament of any one of embodiments Emb. 5, 8, Emb. 17, and Emb. 18, further comprising a pharmaceutically acceptable carrier selected from vehicles, adjuvants, surfactants, suspending agents, emulsifying agents, inert fillers, diluents, excipients, wettings agents, binders, lubricants, buffering agents, disintegrating agents, accessory agents, polymers, polymer matrices, and combinations thereof.

Emb. 20 The medicament of any one of embodiments Emb. 5, 8, Emb. 17-Emb. 19, further comprising a binder selected from starch gelatin, natural sugars, glucose, beta-lactose, corn sweeteners, natural gums, synthetic gums, acacia, tragacanth, sodium alginate, carboxymethylcellulose, polyethylene glycol, waxes, and combinations thereof.

Emb. 21 The medicament of any one of embodiments Emb. 5, 8 and Emb. 17-Emb. 20, further comprising a lubricant selected from sodium oleate, sodium stearate, magnesium stearate, sodium benzoate, sodium acetate, and combinations thereof.

Emb. 22 The medicament of any one of embodiments Emb. 5, 8 and Emb. 17-Emb. 21, further comprising a disintegrator selected from starch, methyl cellulose, agar, bentonite, xanthum gum, and combinations thereof.

Emb. 23 The medicament of any one of embodiments Emb. 5, 8 and Emb. 17-Emb. 22, further comprising an excipient selected from lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and combinations thereof.

Emb. 24 The medicament of any one of embodiments Emb. 5, 8 and Emb. 17-Emb. 23, further comprising a compound selected from sucrose, acacia, tragacanth, gelatin, glycertin, and combinations thereof.

Emb. 25 The medicament of any one of embodiments Emb. 5, 8 and Emb. 17-24, further comprising a diluent comprising water, saline, alcohol, and combinations thereof.

Emb. 26 The medicament of any one of embodiments Emb. 5, Emb. 17-Emb. 25, further comprising a dispersing agent selected from synthetic gum, natural gum, tragacanth, acacia, methylcellulose, glycerin, and combinations thereof.

Emb. 27 The medicament of any one of embodiments, Emb. 5, 8, Emb. 17-Emb. 26 further comprising a coloring agent.

Emb. 28 The medicament of any one of embodiments Emb. 5, 8, Emb. 17-Emb. 27, wherein the medicament comprises an oral solid form selected from a tablet, a capsule, a sachet, a lozenge, a troche, a pill, a powder, a granule, and combinations thereof.

Emb. 29 The medicament of any one of embodiments 5, 9, and 17-28 wherein the medicament comprises an oral liquid form selected from a tincture, solution, suspension, elixir, syrup, and combinations thereof.

Emb. 30 The medicament of any one of embodiments 5, 9, and 17-29, comprising a solution selected from an aqueous solution, a non-aqueous solution, an isotonic sterile injection solution, an anti-oxidant solution, a buffer solution, a bacteriostatic solution, a solution that renders the medicament isotonic with blood of the subject, and combinations thereof.

Emb. 31 The medicament of any one of embodiments 5, 9, and 17-30, comprising a suspension selected from a suspending agent, a solubilizer, a thickening agent, a stabilizer, a preservative, and combinations thereof.

Emb. 32 The medicament of any one of embodiments 5, 9, and 17-31, comprising a diluent selected from a sterile liquid, a mixture of liquids, water, saline, a sugar solution, aqueous dextrose, an alcohol, ethanol, isopropanol, hexadecyl alcohol, glycols, propylene glycol, polyethylene glycol, poly(ethyleneglycol) 400, glycerol ketal, 2,2-dimethyl-1,3-dioxolane-4-methanol, an ether, an oil, a fatty acid, a fatty acid ester, a fatty acid glyceride, an acetylated fatty acid glyceride, a surfactant, a soap, a detergent, a suspending agent, pectin, carbomer, methylcellulose, hydroxypropylmethylcellulose, carboxymethylcellulose, an emulsifying agent, and combinations thereof.

Emb. 33 The medicament of any one of embodiments any one of embodiments 5, 9, and 17-32, comprising an oil selected from a petroleum oil, an animal oil, a vegetable oil, a synthetic oil, and combinations thereof.

Emb. 34 The medicament of any one of embodiments 5, 9, and 17-33, comprising an oil selected from peanut oil, soybean oil, sesame oil, cottonseed oil, corn oil, petrolatum oil, mineral oil, and combinations thereof.

Emb. 35 The medicament of any one of embodiments 5, 9, and 17-34, comprising a fatty acid selected from polyethylene sorbitan, fatty acid esters, sorbitan monooleate, high molecular weight adducts of ethylene oxide with a hydrophobic base, oleic acid, stearic acid, isostearic acid, and combinations thereof.

Emb. 36 The medicament of any one of embodiments 5, 9, and 17-35, comprising a fatty acid ester is selected from ethyl oleate, isopropyl myristate, and combinations thereof.

Emb. 37 The medicament of any one of embodiments 5, 9, and 17-36, comprising a soap selected from fatty alkali metal, ammonium, triethanolamine salts, and combinations thereof.

Emb. 38 The medicament of any one of embodiments 5, 9, and 17-37, comprising a detergent selected from a cationic detergent, a anionic detergent, a nonionic detergent, a amphoteric detergent, and combinations thereof.

Emb. 39 The medicament of any one of embodiments 5, 9, and 17-38, comprising a cationic detergent is selected from dimethyldiakylammonium halides, alkylpyridinium halides, and combinations thereof. Emb. 40 any one of embodiments 5, 9, and 17-39, comprising an anionic detergent selected from alkyl sulfonate, aryl sulfonate, olefin sulfonate, alkyl sulfate, olefin sulfate, ether sulfate, monoglyceride sulfate, sulfosuccinates, and combinations thereof.

Emb. 41 The medicament of any one of embodiments 5, 9, and 17-40, comprising a nonionic detergent selected from fatty amine oxide, fatty acid alkanolamide, polyoxyethylene polypropylene copolymer, and combinations thereof.

Emb. 42 The medicament of any one of embodiments 5, 9, and 17-41, comprising an amphoteric detergent is selected from alkylbetaaminopropionate, 2-alkylimidazoline quaternary ammonium salt, and combinations thereof.

Emb. 43 The medicament of any one of embodiments 5, 9, and 17-42, comprising a surfactant that comprises one or more nonionic surfactants having a hydrophile-lipophile balance (HLB) of from about 12 to about 17.

Emb. 44 The medicament of any of the preceding embodiments, wherein the medicament comprises a topical form selected from an ointment, a cream, a paste, an emulsion, and combinations thereof.

Emb. 45 The medicament of any of the preceding embodiments, wherein the medicament is admixed with a carrier material selected from an alcohol, aloe vera gel, allantoin, glycerine, vitamin A, oil, vitamin E oil, mineral oil, PPG2 myristyl propionate or combinations thereof to form a solution selected from an alcoholic solution, a topical cleaner, a cleansing cream, a skin gel, a skin lotion, a shampoo cream, a shampoo gel, and combinations thereof.

Emb. 46 The medicament of embodimentany of the preceding embodiments, comprising a skin exfoliant, a dermal abrasive, or combinations thereof.

Emb. 47 The medicament of any of the preceding embodiments, comprising a liposome delivery system selected from a small unilamellar vesicle, a large unilamellar vesicle, multilamellar vesicles, and combinations thereof.

Emb. 48 The medicament of any of the preceding embodiments, comprising a liposome delivery system, wherein the liposome delivery system comprises a phospholipid selected from cholesterol, stearylamine, phosphatidylcholine, and combinations thereof.

Emb. 49 The medicament of any of any of the preceding embodiments, comprising a monoclonal antibody.

Emb. 50 The medicament of any of the preceding embodiments, comprising a polymer selected from polyvinyl-pyrrolidone, pyran copolymer, polyhydroxypropylmethacryl-amidephenol, polyhydroxyethylaspartamidephenol, polyethyleneoxidepolylysine substituted with palmitoyl residue, and combinations thereof.

Emb. 51 The medicament of any of the preceding embodiments, comprising a biodegradable polymer selected from polylactic acid, polyepsilon caprolactone, polyhydroxy butyric acid, polyorthoester, polyacetals, polydihydro-pyrans, polycyanoacrylates, cross-linked or amphipathic block copolymers of hydrogels, and combinations thereof.

Emb. 52 A method of modulating the function of fibroblast growth factor 23 (FGF23) in a subject, comprising: administering a soluble α-klotho (sKL) compound to the subject.

Emb. 53 The method of any one of embodiments 1-2 and 52, wherein the sKL compound is administered at a concentration selected from about 50, 60, 70, 75, 80, 85, 90, 95, 100, 110, 120, 130, 140, 150, 160 170, 180, 190, 200, 210, 220, 230, 240, 250, 260, 270, 280, 290, 300, 310, 320, 330, 340, 350, 360, 370, 380, 390, 400, 500, 600, 700, 800, 900, and 1000 nM.

Emb. 54 The method of any one of embodiments 1-2 and 52-53, wherein the sKL compound is administered at a concentration selected from at least about 50, 60, 70, 75, 80, 85, 90, 95, 100, 110, 120, 130, 140, 150, 160 170, 180, 190, 200, 210, 220, 230, 240, 250, 260, 270, 280, 290, 300, 310, 320, 330, 340, 350, 360, 370, 380, 390, 400, 500, 600, 700, 800, 900, and 1000 nM.

Emb. 55 The method of any one of embodiments 1-2 and 54, wherein the sKL compound is administered at a concentration selected from at most about 50, 60, 70, 75, 80, 85, 90, 95, 100, 110, 120, 130, 140, 150, 160 170, 180, 190, 200, 210, 220, 230, 240, 250, 260, 270, 280, 290, 300, 310, 320, 330, 340, 350, 360, 370, 380, 390, 400, 500, 600, 700, 800, 900, and 1000 nM.

Emb. 56 A method of reducing or eliminating hypertrophy in a cardiac myocyte associated with fibroblast growth factor 23 (FGF23), the method comprising exposing the cardiac myocyte to an effective amount of a soluble α-klotho (sKL) compound.

Emb. 57 The method of embodiment 56, wherein the effective amount of the sKL compound comprises a concentration selected from about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 ng/ml.

Emb. 58 The method of any one of embodiments 1, 2, and 56-57, wherein the effective amount of the sKL compound comprises a concentration selected from at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 ng/mL.

Emb. 59 The method of any one of embodiments 1, 2, and 56-58, wherein the effective amount of the sKL compound comprises a concentration selected from at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, and 1000 ng/ml.

Emb. 60 A soluble α-klotho (sKL) compound comprising an amino acid sequence having less than 100% sequence identity compared to a wild-type sKL in a region corresponding to positions 34-981 of human klotho or positions 35-982 of mouse klotho.

Emb. 61 A soluble α-klotho (sKL) compound comprising an amino acid sequence having a sequence identity compared to a wild-type sKL in a region corresponding to positions 34-981 of human klotho or positions 35-982 of mouse klotho selected from about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%.

Emb. 62 Any one of the embodiments above, in which the sKL compound has reduced glycosylation compared to wild type.

Emb. 63 Any one of the embodiments above, in which the sKL compound comprises an amino acid sequence having less than 100% sequence identity compared to a wild-type sKL in a region corresponding to positions 34-981 of human klotho or positions 35-982 of mouse klotho.

Emb. 64 Any one of the embodiments above, in which the sKL compound comprises an amino acid sequence having at least 70% sequence identity compared to a wild-type sKL in a region corresponding to positions 34-981 of human klotho or positions 35-982 of mouse klotho.

Emb. 65 Any one of the embodiments above, in which the sKL compound comprises an amino acid sequence having a sequence identity compared to a wild-type sKL in a region corresponding to positions 34-981 of human klotho or positions 35-982 of mouse klotho, wherein said sequence identity level is at least: 75, 80, 85, 90, 95, 96, 97, 99, and 100%.

Emb. 66 Any one of the embodiments above, in which the sKL compound comprises an amino acid sequence having a sequence identity compared to a wild-type sKL in a region corresponding to positions 34-981 of human klotho or positions 35-982 of mouse klotho, wherein the wild-type sKL is selected from Pan troglodytes, Macaca mulatta, Canis lupus familiaris, Bos taurus, Rattus norvegicus, Gallus gallus, Danio rerio, and Xenopus tropicalis.

Emb. 67 Any one of the embodiments above, in which the sKL compound comprises an amino acid sequence having a sequence identity compared to a wild-type sKL in a region corresponding to positions 34-981 of human klotho or positions 35-982 of mouse klotho, wherein the region of the sKL is from one of SEQ ID NO: 1-10.

Emb. 68 Any one of embodiments above, in which the sKL compound comprises a mutation that reduces the glycosylation of the sKL compound compared to wild type.

Emb. 69 Any one of the embodiments above, in which the sKL compound comprises a mutation that reduces the glycosylation of the sKL compound compared to wild type, and in which the mutation is a substitution at a position that is normally glycosylated.

Emb. 70 Any one of the embodiments above, in which the sKL compound comprises a mutation that reduces the glycosylation of the sKL compound compared to wild type, and in which the mutation is a substitution at an N residue that is normally glycosylated.

Emb. 71 Any one of the embodiments above, in which the sKL compound comprises a mutation that reduces the glycosylation of the sKL compound compared to wild type, and in which the mutation is an N->Q substitution at a residue that is normally glycosylated.

Emb. 72 Any one of embodiment the embodiments above, in which the sKL compound comprises a mutation that increases the binding affinity of the sKL compound to FGF23 compared to wild type.

Emb. 73 Any one of embodiment the embodiments above, in which the sKL compound comprises a mutation that increases stability of the sKL compound to FGF23 compared to wild type.

Emb. 74 Any one of the embodiments above, wherein the sKL compound comprises a substitution compared to the wild-type at one or more amino acids corresponding to the following positions in human or mouse sKL: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, and 982.

Emb. 75 Any one of the embodiments above, wherein the sKL compound comprises a substitution compared to the wild-type at one or more amino acids corresponding to positions 34-982 in human or mouse sKL, and wherein the wild-type sKL is selected from Pan troglodytes, Macaca mulatta, Canis lupus familiaris, Bos taurus, Rattus norvegicus, Gallus gallus, Danio rerio, and Xenopus tropicalis.

Emb. 76 Any one of the embodiments above, wherein the sKL compound comprises a substitution compared to the wild-type at one or more amino acids corresponding to positions 34-982 in human or mouse sKL, wherein the wild-type sKL comprises at least one of SEQ ID NO: 1-10.

Emb. 77 Any one of the embodiments above, in which the sKL compound comprises a mutation that is a substitution at one or more positions corresponding to positions 106, 159, 283, 344, 607, 612, 630, and 694 of human sKL.

Emb. 78 Any one of the embodiments above, in which the sKL compound comprises a mutation that is a substitution at one or more of positions 106, 159, 283, 344, 607, 612, 630, and 694 of SEQ ID NO: 1.

Emb. 79 Any one of the embodiments above, in which the sKL compound comprises a mutation that is a substitution at one or more of positions that correspond to positions 106, 159, 283, 344, 607, 612, 630, and 694 of SEQ ID NO: 1.

Emb. 80 Any one of the embodiments above, in which the sKL compound comprises a mutation that is an N→Q substitution at one or more positions corresponding to positions 106, 159, 283, 344, 607, 612, 630, and 694 of human sKL.

Emb. 81 Any one of the embodiments above, in which the sKL compound comprises a mutation that is an N→Q substitution at one or more of positions 106, 159, 283, 344, 607, 612, 630, and 694 of SEQ ID NO: 1.

Emb. 82 Any one of the embodiments above, in which the sKL compound comprises a mutation that reduces the glycosylation of the sKL compound compared to wild type.

Emb. 83 Any one of the embodiments above, in which the sKL compound comprises a mutation that increases the binding affinity of the sKL compound to FGF23 compared to wild type.

Emb. 84 Any one of the embodiments above, in which the sKL compound comprises a mutation that increases stability of the sKL compound to FGF23 compared to wild type.

Emb. 85 Any one of the embodiments above, in which the sKL compound comprises a mutation that is a substitution at one or more of the following positions: 106, 159, 283, 344, 607, 612, 630, and 694. Emb. 86 Any one of the embodiments above, in which the sKL compound comprises one or more N→Q substitutions at one or more of the following positions: 106, 159, 283, 344, 607, 612, 630, and 694.

Emb. 87 A nucleic acid that encodes any one of the sKL compounds listed above.

Emb. 88 A nucleic acid that is complementary to one that encodes any one of the sKL compounds listed above.

Emb. 89 A nucleic acid that hybridizes under stringent conditions with a nucleic acid that encodes any one of the sKL compounds listed above.

Emb. 90 The nucleic acid of any one of embodiments 87-89, wherein the nucleic acid is selected from RNA, DNA, LNA, BNA, copolymers of any of the foregoing, and analogs thereof.

Emb. 91 A genetically modified cell comprising a nucleic acid of any one of embodiments 87-90.

Emb. 92 A genetically modified cell comprising a nucleic acid of any one of embodiments 87-90, wherein the genetically modified cell is selected from an animal cell, yeast cell, fungal cell, bacterial cell, protist cell, and archaeal cell.

Emb. 93 The genetically modified cells of embodiment 92, wherein the genetically modified cell is selected from a Homo sapiens cell, a Pan troglodytes cell, a Macaca mulatta cell, a Canis lupus familiaris cell, a Bos taurus cell, a Mus musculus cell, a Rattus norvegicus cell, a Gallus gallus cell, a Danio rerio cell, a Xenopus tropicalis cell, an Escherichia coli cell, a Salmonella typhimurium cell, a Pseudomonas fluorescens cell, a Bacillus subtilis cell, a Mycoplasma genitalium cell, a Synechocystis sp. cell, a Dictyostelium discoideum cell, a Tetrahymena thermophila cell, an Emiliania huxleyi cell, a Thalassiosira pseudonana cell, an Aspergillus sp. cell, a Neurospora crassa cell, a Saccharomyces cerevisiae cell, and a Schizosaccharomyces pombe cell.

Emb. 94 A genetically modified animal comprising a nucleic acid of any one of embodiments 87-90.

Emb. 95 A genetically modified animal comprising a nucleic acid of any one of embodiments 87-90, wherein the animal is a non-human animal.

Emb. 96 A genetically modified animal comprising a nucleic acid of any one of embodiments 87-90, wherein the animal is a mouse, rat, other rodent, rabbit, dog, cat, swine, cattle, sheep, horse, or primate.

Emb. 97 A method of making an sKL compound, comprising expressing a nucleic acid of any one of embodiments 87-90.

Emb. 98 A method of making an sKL compound, comprising expressing a nucleic acid of any one of embodiments 87-90 in a microorganism.

Emb. 99 A method of making an sKL compound, comprising expressing a nucleic of any one of embodiments 33-Emb. 90 in a microorganism selected from yeasts, fungi, bacteria, protist, and archaea.

Emb. 100 A method of making an sKL compound, comprising expressing a nucleic acid of any one of embodiments 33-Emb. 90 in a microorganism selected from Escherichia coli, Salmonella typhimurium, Pseudomonas fluorescens, Bacillus subtilis, Mycoplasma genitalium, Synechocystis sp., Dictyostelium discoideum, Tetrahymena thermophila, Emiliania huxleyi, Thalassiosira pseudonana, Aspergillus sp., Neurospora crassa, Saccharomyces cerevisiae, and Schizosaccharomyces pombe.

### CONCLUSIONS

It is to be understood that any given elements of the disclosed embodiments of the invention may be embodied in a single structure, a single step, a single substance, or the like. Similarly, a given element of the disclosed embodiment may be embodied in multiple structures, steps, substances, or the like. The foregoing description and accompanying drawings illustrate and describe certain processes, machines, manufactures, and compositions of matter, some of which embody the invention(s). Such descriptions or illustrations are not intended to limit the scope of what can be claimed, and are provided as aids in understanding the claims, enabling the making and use of what is claimed, and teaching the best mode of use of the invention(s). If this description and accompanying drawings are interpreted to disclose only a certain embodiment or embodiments, it shall not be construed to limit what can be claimed to that embodiment or embodiments. Any examples or embodiments of the invention described herein are not intended to indicate that what is claimed must be coextensive with such examples or embodiments. Where it is stated that the invention(s) or embodiments thereof achieve one or more objectives, it is not intended to limit what can be claimed to versions capable of achieving any or all such objectives. Any statements in this description criticizing the prior art are not intended to limit what is claimed to exclude any aspects of the prior art. Additionally, the disclosure shows and describes certain embodiments of the processes, machines, manufactures, compositions of matter, and other teachings disclosed, but it is to be understood that the teachings of the present disclosure are capable of use in various other combinations, modifications, and environments and is capable of changes or modifications within the scope of the teachings as expressed herein. Any section headings herein are provided only for consistency with the suggestions of 37 C.F.R. § 1.77 or otherwise to provide organizational queues. These headings shall not limit or characterize the invention(s) set forth herein.

TABLE 5
ALIGNMENT OF CANONICAL KL PROTEINS

| | | | |
|---|---|---|---|
| NP_004786.2 | 1 | MPASAPPRRPR---PPPPSLSL--L-LVLLGLGGRRLRA-EPGDGAQTWA | 43 |
| XP_522655.2 | 1 | MPASAPPRRPR---PPPPSLSL--L-LVLLALGGRRLRA-EPGDGAQTWA | 43 |
| XP_001101127.1 | 1 | MPASAPPRRLR---PPPPSLSLSLL-LVLLALGGRRLRA-EPGDGAQTWA | 45 |
| XP_003433357.1 | 1 | MATCILQMR-------------------FLRLGKILFHS-SP-------- | 22 |
| NP_001178124.1 | 1 | MPARAPPRRLRAFPPPPPPL----W-LLLLALGGRPLRA-EPGDGAQTWA | 44 |
| NP_038851.2 | 1 | MLARAPPRRP----PRLVLLRLLLLHLLLLALRARCLSA-EPGQGAQTWA | 45 |
| NP_112626.1 | 1 | MPARAPPRRL----PRLLLLRLLSLHLLLLTLRARCLSA-EPGQGAQTWA | 45 |
| XP_004938814.1 | 1 | ---MAP--------PPPPVLPVLPVLLVLLGLG-RPLLG-APGQGAGTWA | 37 |
| XP_690797.4 | 1 | -------------------MKVTWIPLPLVLFCQFQGTASDPGAGQHTWD | 31 |
| XP_002934067.1 | 1 | --------------MRPGALALLWAAAWGCALCALCGNA-EK----NVWS | 31 |
| NP_004786.2 | 44 | RFSRPPAPEAAGLFQGTFPDGFLWAVGSAAYQTEGGWQQHGKGASIWDTF | 93 |
| XP_522655.2 | 44 | RFSRPPAPEAAGLFQGTFPDGFLWAVGSAAYQTEGGWQQHGKGASIWDTF | 93 |
| XP_001101127.1 | 16 | RFARPPAPEAAGLFQGTFPDGFLWAVGSAAYQTEGGWQQHGKGASIWDTF | 95 |
| XP_003433357.1 | 23 | --------------------------------QSTGG------------- | 27 |
| NP_001178124.1 | 45 | RFARPPTPEAAGLLHDTFPDGFLWAVGSAAYQTEGGWQQHGKGASIWDTF | 94 |
| NP_038851.2 | 46 | RFARAPAPEAAGLLHDTFPDGFLWAVGSAAYQTEGGWRQHGKGASIWDTF | 95 |
| NP_112626.1 | 46 | RFARPPVPEASGLLHDTFPDGFLWAVGSAAYQTEGGWRQHGKGASIWDTF | 95 |
| XP_004938814.1 | 38 | RFAHLPYPQDQLFLHDTFPDGFLWGAGSAAYQTEGGWRQGGKGASIWDTF | 8 |
| XP_690797.4 | 32 | TFSKLPYPDDKAFLYDTFPDKFMWAVGTAAYSVEGAWEKDGKGKSIWDTF | 81 |
| XP_002934067.1 | 32 | RFAQLPFPQDNLFLYGTFPPNFMWSVGTAAYQVEGGWEQNGKAPSIWDTF | 81 |
| NP_004786.2 | 94 | THHPLAPPGDSRNASLPLGAPSPLQPATGDVASDSYNNVERDTEALRELG | 143 |
| XP_522655.2 | 94 | THHPLAPPGDSRNASLPSGAPSPLQPATGDVASDSYNNVFRDTEALRELG | 143 |
| XP_001101127.1 | 96 | THHPLAPPGDSRIANVPSGAPSPLQPATGDVASDSYNNVFRDTEALRELG | 145 |
| XP_003433357.1 | 28 | ----------------SGGTRGPRAPAQ-----------------LRT-- | 42 |
| NP_001178124.1 | 95 | THRPPAPPGDPSAAGWPSGAPSPPPPATGDVASDGYNNVERDTEGLRELG | 144 |
| NP_038851.2 | 96 | THHSGAAPSDSPIVVAPSGAPSPPLSSTGDVASDSYNNVYRDTEGLRELG | 145 |
| NP_112626.1 | 96 | THHPRAIPEDSPIVMAPSGAPLPPLPSTGDVASDSYNNVYRDTEGLRELG | 145 |
| XP_004938814.1 | 88 | THRPTT----------PAGSILP-GPTGGDVASDSYNNIFRDIEGLRHLG | 126 |
| XP_690797.4 | 82 | TRGGTR-------------------VSRGDVGSDSYHNIPGDLRALQQLG | 112 |
| XP_002934067.1 | 82 | CHKSGQ------------------LDATGDVASDSYNNLFRDTEALKSLG | 113 |
| NP_004786.2 | 144 | VTHYRFSISWARVLPNGSAGVPNREGLRYYRRLLERLRELGVQPVVTLYH | 193 |
| XP_522655.2 | 144 | VTHYRFSISWARVLPNGSAGVPNREGLRYYRRLLERLRELGVQPVVTLYH | 193 |
| XP_001101127.1 | 146 | VTHYRESISWARVLPNGSAGVPNREGLRYYRRLLERLRELGVQPVVTLYH | 195 |
| XP_003433357.1 | 43 | --------------QRGTDKLVAKSELK---------------------- | 56 |
| NP_001178124.1 | 145 | VTHYRFSISWARVLPNGSASAPNREGLRYYRRLLERLRELGVQPVVTLYH | 194 |
| NP_038851.2 | 146 | VTHYRESISWARVLPNGTAGTPNREGLRYYRRLLERLRELGVQPVVTLYH | 195 |
| NP_112626.1 | 146 | VTHYRFSISWARVLPNGTAGTPNREGLRYYRRLLERLRELGVQPVVTLYH | 195 |
| XP_004938814.1 | 127 | VSHYRFSLAWTRLMPNGTAPV-NPVGLAHYGQVLSRLRELGIEPIVTLYH | 175 |
| XP_690797.4 | 113 | VSHYRFSLSWPRIFSNGTKESYNDKGVEYYKNLIRGLKDIKVQPVVTLYH | 162 |
| XP_002934067.1 | 114 | VTHYRFSISWPRLFPNGTESAPNEAGLSYYRNLILRLKELRIEPVVTLYH | 163 |
| NP_004786.2 | 194 | WDLPQRLQDAYGGWANRALADHFRDYAELCFRHFGGQVKYWITIDNPYVV | 243 |
| XP_522655.2 | 194 | WDLPQRLQDAYGGWANRALADHFRDYAELCFRHFGGQVKYWITIDNPYVV | 243 |
| XP_001101127.1 | 196 | WDLPQRLQDAYGGWANRALADHFRDYAELCFRHFGGQVKYWITIDNPYVV | 245 |
| XP_003433357.1 | 57 | -----------AKTAHRALADHFRDYAELCFRHFCGQVKYWITIDNPYVV | 95 |
| NP_001178124.1 | 195 | WDLPQRLQDAYGGWANRALADHFRDYAELCFRHFGGQVKYWITIDNPYVV | 244 |
| NP_038851.2 | 196 | WDLPQRLQDTYGGWANRALADHFRDYAELCFRHFGGQVKYWITIDNPYVV | 245 |
| NP_112626.1 | 196 | WDLPQRLQDTYGGWANRALADHFRDYAELCFRHFGGQVKYWITIDNPYVV | 245 |
| XP_004938814.1 | 176 | WDLPQGLQDAFGGWASPVLPNLFHDYAELCFRHFGGQVRYWLTMDNPYVV | 225 |
| XP_690797.4 | 163 | WDLPDSLQTLFGGWSNSVMVELFRDYADFCFKTFGSDVKFWITIDNPFVV | 212 |
| XP_002934067.1 | 164 | WDLPQRLQDVYGGWVSESMVGIFRDYAEACFRLLGDEVKYWITIDNPYVV | 213 |
| NP_004786.2 | 244 | AWHGYATGRLAPGIRGSPRLGYLVAHNLLL-------------------- | 273 |
| XP_522655.2 | 244 | AWHGYATGRLAPGIRGSPRLGYLVAHNLLL-------------------- | 273 |
| XP_001101127.1 | 246 | AWHGYATGRLAPGIRGSPRLGYLVAHNLLL-------------------- | 275 |
| XP_003433357.1 | 96 | AWHGYATGRLAPGVRGSPRLGYLVAHNLLL-------------------- | 125 |
| NP_001178124.1 | 245 | AWHGYATGRLAPGVRGSPRLGYLVAHNLLL-------------------- | 274 |
| NP_038851.2 | 246 | AWHGYATGRLAPGVRGSSRLGYLVAHNLLL-------------------- | 275 |
| NP_112626.1 | 246 | AWHGYATGRLAPGVRGSSRLGYLVAHNLLL-------------------- | 275 |
| XP_004938814.1 | 226 | AWHGYGTGRLPPGVQGGPSLGYRAAHHLLQSRCTAVGALFGTLCIVTLKA | 275 |
| XP_690797.4 | 213 | AWHGYGTGVVAPGIKNDSDLPFRVGHNLLK-------------------- | 242 |
| XP_002934067.2 | 214 | AWQGYATGKVPPGIKGDKVMGYRAGHNIIK-------------------- | 243 |
| NP_004786.2 | 274 | ------AHAKVWHLYNTSFRPTQGGQVSIALSSHWINPRRMTDHSIKECQ | 317 |
| XP_522655.2 | 274 | ------AHAKVWHLYNTSFRPTQGGQVSIALSSHWINPRRMTEHSIKECQ | 317 |
| XP_001101127.1 | 276 | ------AHAKVWHLYNTSFRPTQGGQVSIALSSHWINPRRMTDHSIKECQ | 319 |
| XP_003433357.1 | 126 | ------AHAKIWHLYNTSFRPTQGGQVSIALSSHWINPRRMTDHSIKECQ | 169 |
| NP_001178124.1 | 275 | ------AHAKIWHLYDTSFRPTQGGQVSIALSSHWISPRRMTEHSIQECQ | 318 |
| NP_038851.2 | 276 | ------AHAKVWHLYNTSFRPTQGGRVSIALSSHWINPRRMTDYNIRECQ | 319 |
| NP_112626.1 | 276 | ------AHAKVWRLYNTSFRPTQGGRVSIALGSHWITPRRMTDYHIRECQ | 319 |
| XP_004938814.1 | 276 | VGLHTRAHAKVWHLYNDHFRPTQKGKVSIALSSHWIKPQHMTEKNIKECQ | 325 |
| XP_690797.4 | 243 | ------AHAAAWHLYDERYRAAQGGRVSMALGSHWIKPSRTRQESRKACQ | 286 |
| XP_002934067.1 | 244 | ------AHAAVWHLYDKKFRPQQGGQISIALASHWINPVNMTSHDIGDCQ | 287 |
| NP_004786.2 | 318 | KSLDFVLGWFAKPVFIDGDYPESMKNNLSSILPDFTESEKKFIKGTADFF | 367 |
| XP_522655.2 | 318 | KSLDFVLGWFAKPVFIDGDYPESMKNNLSSILPDFTESEKKFIKGTADFF | 367 |
| XP_001101127.1 | 320 | KSLDFVLGWFAKPIFIDGDYPESMKNNLSSLLPDFTESEKKFIKGTADFF | 369 |
| XP_003433357.1 | 170 | KSLDFVLGWFAKPIFIDGDYPESMKNNLSSLLPVFTESEKKFIKGTADFF | 219 |
| NP_001178124.1 | 319 | KSLDFVLGWFAKPIFIDGDYPESMKNNLSSLLPDFTESEKKFIKGTADFF | 368 |
| NP_038851.2 | 320 | KSLDFVLGWFAKPIFIDGDYPESMKNNLSSLLPDFTESEKRLIRGTADFF | 369 |
| NP_112626.1 | 320 | KSLDFVLGWFAKPIFIDGDYPKSMKNNLSSLLPDFTESEKRFIRGTADFF | 369 |
| XP_004938814.1 | 326 | KSLDFVLGWFAKPIFINGDYPESMRSNLSSLLPEFSEEDKKYIKGTADFF | 375 |
| XP_690797.4 | 287 | RSLNFVLGWFARPLFVDGDYPPCMKDNLTHRLPSFTEAESAYVNGTADFF | 336 |
| XP_002934067.1 | 288 | KSLDFALGWFAKPIFIDGDYPQTMKNNLSSLLPEFSDQEKKLNKGTADFF | 337 |
| NP_004786.2 | 368 | ALCFGPTLSFQLLDPHMKFRQLESPNLRQLLSWIDLEFNHPQIFIVENGW | 417 |
| XP_522655.2 | 368 | ALSFGPTLSFQLLDPHMKFRQLESPNLRQLLSWIDLEFNHPQIFIVENGW | 417 |
| XP_001101127.1 | 370 | ALSFGPTLSFQLLDPHMKFRQLESPSLRQLLSWIDLEYNHPQIFIVENGW | 419 |
| XP_003433357.1 | 220 | ALSFGPTLSFQLLDPHMKFHQLESPSLRQLLSWIDLEYNHPQIFIVENGW | 269 |
| NP_001178124.1 | 369 | ALSFGPTLSFQLLDPQMKFRQLESPSLRQLLSWIDLEYNHPQIFIVENGW | 418 |
| NP_038851.2 | 370 | ALSFGPTLSFQLLDPNMKFRQLESPNLRQLLSWIDLEYNHPPIFIVENGW | 419 |
| NP_112626.1 | 370 | ALSFGPTLSFQLLDPSMKFRQLESPSLRQLLSWIDLEYNHPQIFIVENGW | 419 |
| XP_004938814.1 | 376 | ALSFGATLSFQLLDSHMKFQQLESISLRQLLYWISTEYNNPPVFIVENSW | 425 |
| XP_690797.4 | 337 | ALSHGPALSFQLINDSLRFGQTEDLGLRMLLYWVRAEYNNPPIFVVESGW | 386 |
| XP_002934067.1 | 338 | SLSFGPNLSFQMLDPDMKFRQVESTSLRKILYWINEQYNKPRIFIVENSW | 387 |
| NP_004786.2 | 418 | FVSGTTKRDDAKYMYYLKKFIMETLKAIKLDGVDVIGYTAWSLMDGFEWH | 467 |
| XP_522655.2 | 418 | FVSGTTKRDDAKYMYYLKKFIMETLKAIKLDGVDVIGYTAWSLMDGFEWH | 467 |
| XP_001101127.1 | 420 | FVSGTTKRDDAKYMYYLKKFIMETLKAIKLDGVDVIGYTAWSLMDGFEWH | 469 |
| XP_003433357.1 | 270 | FVSGTTKRDDAKYMYYLKKFIMETLKAIRLDGVDVIGYTAWSLMDGFEWH | 319 |
| NP_001178124.1 | 419 | FVSGTTKRDDAKYMYYLKKFIMETLKAIRLDGVDVIGYTAWSLMDGFEWH | 468 |
| NP_038851.2 | 420 | FVSGTTKRDDAKYMYYLKKFIMETLKAIRLDGVDVIGYTAWSLMDGFEWH | 469 |
| NP_112626.1 | 420 | FVSGTTRRDDAKYMYYLKKFIMESLKAIRLDGVDVIGYTAWSLMDGFEWH | 469 |
| XP_004938814.1 | 426 | FVSGSTKRDDAKYIYYLKKFIMETLKAIRYDGVNVFGYTVWSLLDGFEWH | 475 |
| XP_690797.4 | 387 | YGSGNTKTKDAKHMYYLKRFIMETLKAIHVDRVNVIGYTAWSLLDGYEWY | 436 |
| XP_002934067.1 | 388 | FLSGNTKREDAKYMYYLKKFVMETLKAIKYDGVKVIGYTAWSLMDGFEWL | 437 |
| NP_004786.2 | 468 | RGYSIRRGLFYVDFLSQDKMLLPKSSALFYQKLIEKNGFPPLPENQPLEG | 517 |
| XP_522655.2 | 468 | RGYSIRRGLFYVDFLSQDKMLLPKSSALFYQKLIEKNGFPPLPENQPLEG | 517 |
| XP_001101127.1 | 470 | RGYSIRRGLFYVDFLSQEKTLLPKSSALFYQKLIEKNGFPPLPENQPLEG | 519 |
| XP_003433357.1 | 320 | RGYSIRRGLFYVDFLSQDKKLLPKSSALFYQKLIEKNGFPPLPENQPLEG | 369 |
| NP_001178124.1 | 469 | RGYSIRRGLFYVDFLSQDKKLLPKSSALFYQKLIENNGFPPLPENQPLEG | 518 |
| NP_038851.2 | 470 | RGYSIRRGLFYVDFLSQDKELLPKSSALFYQKLIEDNGFPPLPENQPLEG | 519 |
| NP_112626.1 | 470 | RGYSIRRGLFYVDFLSQDKELLPKSSALFYQKLIENNGFPPLPENQPLEG | 519 |
| XP_004938814.1 | 476 | RGYSIRRGLFYVDFQSHDKKLIPKSSVLFYQKLIEKNGFPPLPENQPIAG | 525 |
| XP_690797.4 | 437 | REYAIRRGLFYVDENTPDLKREPKASATFYSKLIEKNGFPQLPENRPAQG | 486 |
| XP_002934067.1 | 438 | REYTIRRGLFYVDFTSHNKKLMPKSSALFYQQLIKMNGFPPLPENQPVEG | 487 |
| NP_004786.2 | 518 | TFPCDFAWGVVDNYIQVDTTLSQFTDLNVYLWDVHHSKRLIKVDGVVTK- | 566 |
| XP_522655.2 | 518 | TFPCDFAWGVVDNYIQVDTTLSQFTDLNVYLWDVHHSKRLIKVDGVVTK- | 566 |
| XP_001101127.1 | 520 | TFPCDFAWGIVDNYIQVDTTLSQFTDLNVYLWDVHHSKRLIKVDGVVTK- | 568 |
| XP_003433357.1 | 370 | TFPCDFAWGIVDNYIQVDTTLSQFTDPNVYLWDVHHSKRLIKVDGLRAK- | 418 |
| NP_001178124.1 | 519 | TFPCDFAWGVVDNCIQVDTTLSQFIDPNVYLWDVHRSKRLIKVDGVLTK- | 567 |
| NP_038851.2 | 520 | TFPCDFAWGVVDNYVQVDTTLSQFTDPNVYLWDVHHSKRLIKVDGVVAK- | 568 |
| NP_112626.1 | 520 | TFPCDFAWGVVDNYIQVDPTLSQFTDPNVYLWDVHHSKRLIKVDGVVAK- | 568 |
| XP_004938814.1 | 526 | VFPCGFAWGIVDNYIQVDTTPAQFLDPNVYVWDVHQTKKLIKVDGVFTS- | 574 |
| XP_690797.4 | 487 | AFPCDFAWGVAANSIQVDTTPTQFTDTNVYVWNISGNGELKKLPGLQAPH | 536 |
| XP_002934067.1 | 488 | TFPCDFAWGTSAYRIHIDTTPSQFNDPNVYVWDMNKTKGLTKVEGITVP- | 536 |
| NP_004786.2 | 567 | -KRKSYCVDFAAIQPQIALLQEMHVTHFRFSLDWALILPLGNQSQVNHTI | 615 |
| XP_522655.2 | 567 | -KRKSYCVDFAAIQPQIALLQEMHITHFRFSLDWALILPLGNQSQVNHTI | 615 |
| XP_001101127.1 | 569 | -KRKSYCVDFAAIQPQITLLQEMHVTHFRFSLDWALILPLGNQSQVNHTI | 617 |
| XP_003433357.1 | 419 | -KRKPYCVDFAAIGPQVALLQEMHVSHFHFSLDWALLLPLGNQSRVNHAA | 467 |
| NP_001178124.1 | 568 | -TRKSYCVDFAAIRPQIALLQEMHVTHFHFSLDWALILPLGNRSQVNRTV | 616 |
| NP_038851.2 | 569 | -KRKPYCVDFSAIRPQITLLREMRVTHFRFSLDWALILPLGNQTQVNHTV | 617 |
| NP_112626.1 | 569 | -KRKPYCVDFSAIRPQITLLREMRVTHFRFSLDWALILPLGNQTQVNRTV | 617 |
| XP_004938814.1 | 575 | -QRKHHCVDFAAIRLQISLLQEMHVTHFHFSLKWSSVLPLGNLSLINHTL | 623 |
| XP_690797.4 | 537 | LRRTPHCADYGSIRQQVSDLLRMQVSHFHFSLNWSSIVPTGHVSDANETL | 586 |
| XP_002934067.1 | 537 | -KRKTQCVDFASIRQQISMLREIHITHFYFALKWAAILPLGNLSLIHHKV | 585 |
| NP_004786.2 | 616 | LQYYRCMASELVRVNITPVVALWQPMAPNQGLPRLLARQGAWENPYTALA | 665 |
| XP_522655.2 | 616 | LQYYRCMASELVRVNITPVVALWQPMAPNQGLPRLLARQGAWENPYTALA | 665 |
| XP_001101127.1 | 618 | LQYYRCMVSELVRVNITPVVALWQPVAPNQGLPRLLARQGAWENPYTALA | 667 |
| XP_003433357.1 | 468 | LHYYGCVASELLRANITPVVALWRPAAAHQGLPGPLAQRGAWENPRTALA | 517 |
| NP_001178124.1 | 617 | LGFYRCVASELVRANITPVVALWRPAAPHQGLPAPLARHGAWENPHTALA | 666 |
| NP_038851.2 | 618 | LHFYRCMISELVHANITPVVALWQPAAPHQGLPHALAKHGAWENPHTALA | 667 |
| NP_112626.1 | 618 | LHFYRCMVSELVHANITPVVALWQPATPHQGLPHALAKHGAWENPHTALA | 667 |
| XP_004938814.1 | 624 | VHYYQCFASELLRVNITPVVALWQPMAENQELPTSLAKFGAWENSETVQA | 673 |
| XP_690797.4 | 587 | LRYYYCFVSELQKVNITPVVTLWHHTGKLSSLPAPMEASDGWQSEKTVQA | 636 |
| XP_002934067.1 | 586 | LHYYQCFVSELVRVNITPVVALWQPLAENQGLPIDLAKNGGWVNYHTVSA | 635 |
| NP_004786.2 | 666 | FAEYARLCFQELGHHVKLWITMNEPYTRNMTYSAGHNLLKAHALAWHVYN | 715 |
| XP_522655.2 | 666 | FAEYARLCFQALGHHVKLWITMNEPYTRNMTYSAGHNLLKAHALAWHVYN | 715 |
| XP_001101127.1 | 668 | FAEYARLCFQELGHHVKLWITMNEPYTRNMTYSAGHNLLKAHALAWHVYN | 717 |
| XP_003433357.1 | 518 | FAEYARLCFRALGRHVKVWITLREPPTRNLTLRAGHNLLRAHALAWRVYD | 567 |
| NP_001178124.1 | 667 | FAEYASLCFQDLGRHVKFWITMHEPSTRNMTYSAGHNLLKAHALAWRTYD | 716 |
| NP_038851.2 | 668 | FADYANLCFKELGHWVNLWITMNEPNTRNMTYRAGHHLLRAHALAWHLYD | 717 |
| NP_112626.1 | 668 | FADYANLCFEELGHWVKFWITINEPNSRNMTYRAGHHLLKAHALAWHLYD | 717 |
| XP_004938814.1 | 674 | FVEYAKFCFASLGDHVKFWITMNEPSVKNLTYTAGHNLLRAHAKAWHLYD | 723 |
| XP_690797.4 | 637 | FVDYARLCFQRLGAHVKLWITLNEPNDEDLEYTVGHQLLRAHALAWHVYD | 686 |
| XP_002934067.1 | 636 | FVEYARLCFKELGNYVGMWITMNEPSMRNLTYAAGHNLLKAHALAWHLYD | 685 |
| NP_004786.2 | 716 | EKFRHAQNGKISIALQADWIEPACPFSQKDKEVAERVLEFDIGWLAEPIF | 765 |
| XP_522655.2 | 716 | EKFRHAQNGKISIALQADWIEPACPFSQKDKEVAERVLEFDIGWLAEPIF | 765 |
| XP_001101127.1 | 718 | EKFRHAQNGKISIALQADWIEPACPFSQKDKEVAERVLEFDIGWLAEPIF | 767 |
| XP_003433357.1 | 568 | EQFRGSQQGKVSIALQADWVEPACPSSQKDREVAERVLEFDVGWLAEPIF | 617 |
| NP_001178124.1 | 717 | ERFRRSQKGKISIALQADWIEPACPFSPEDREVAERVLEFDIGWLAEPIF | 766 |
| NP_038851.2 | 718 | DKFRAAQKGKISIALQADWIEPACPFSQNDKEVAERVLEFDIGWLAEPIF | 767 |
| NP_112626.1 | 718 | DKFRAAQKGKISIALQVDWIEPACPFSQKDKEVAERVLEFDVGWLAEPIF | 767 |
| XP_004938814.1 | 724 | KEFRRSQKGKISIALQADWVEPACPFSRNDQEVADRILEFDIGWLAEPIF | 773 |
| XP_690797.4 | 687 | REFRKAQGGKASLVLHMDWVEPAFSFNREDVAPADRVLDFRVGWFAEPIF | 736 |
| XP_002934067.1 | 686 | RDFRKAQKGQISIAVQADWVEPASPFSKNDKETSRRILEFEIGRLSDPIF | 735 |
| NP_004786.2 | 766 | GSGDYPWVMRDWLNQRN-----NFLLPYFTEDEKKLIQGTFDFLALSHYT | 810 |
| XP_522655.2 | 766 | GSGDYPWVMRDWLNQRN-----NFLLPYFTEDEKKLIQGTFDFLALSHYT | 810 |
| XP_001101127.1 | 768 | GSGDYPWVMRDWLNQRN-----NFLLPYFTEDEKKLIQGTFDFLALSHYT | 812 |
| XP_003433357.1 | 618 | GSGDYPRLMRDWLTRRD-----HSLLPYFTDEEKRLIRGSFDFLALSHYT | 662 |
| NP_001178124.1 | 767 | GSGDYPRVMRDWLNQRN-----NFLLPYFTDEEKKLIRGSFDFLALSHYT | 811 |
| NP_038851.2 | 768 | GSGDYPRVMRDWLNQKN-----NFLLPYFTEDEKKLVRGSFDFLAVSHYT | 812 |
| NP_112626.1 | 768 | GSGDYPHVMREWLNQKN-----NFLLPYFTEDEKKLIRGSFDFLALSHYT | 812 |
| XP_004938814.1 | 774 | GSGDYPHMMRAWLHQRNSVDLYNFHLPSFSEDEKKLIQGSFDFFALSHYT | 823 |
| XP_690797.4 | 737 | GKGDYPAVMRSWLQQRNTIDLFNYHLPTFSEEDRLLVKGTYDFFAISHFT | 786 |
| XP_002934067.1 | 736 | LSGDYPKVMRDWLAPRNNLDVFEFYLPSFTEEEKNLIQGTFDFFALSHFT | 785 |
| NP_004786.2 | 811 | TILVDSEKEDPIKYNDYLEVQEMTDITWINSPSQVA-VVPWGLRKVLNWL | 859 |
| XP_522655.2 | 811 | TILVDSEKEDPIKYNDYLEVQEMTDITWINSPSQVA-VVPWGLRKVLNWL | 859 |
| XP_001101127.1 | 813 | TILVDSEKEDPIKYNDYLEVQEMTDITWINSPSQVA-VVPWGLRKVLNWL | 861 |
| XP_003433357.1 | 663 | TILVDWEKEDPVKYNDYLEVQEMTDITWINSPSQVA-VVPWGLRKVLNWL | 711 |
| NP_001178124.1 | 812 | TILVDWEKEDPIKYNDYLAVQEMTDITWINSPSQVA-VVPWGLRKVLNWL | 860 |
| NP_038851.2 | 813 | TILVDWEKEDPMKYNDYLEVQEMTDITWLNSPSQVA-VVPWGLRKVLNWL | 861 |
| NP_112626.1 | 813 | TILVDWEKEDPIKYNDYLEVQEMTDITWLNSPNQVA-VVPWGLRKALNWL | 861 |
| XP_004938814.1 | 824 | TILVGWEKEDALKYDHYLEVQMISDITWLHSPSRAA-VVPWGLRKVLRWV | 872 |
| XP_690797.4 | 787 | TSMVYDGVEDKYTFKDKLQVQLISDVTWIMSPRRNSPVVPWGLRKALNWV | 836 |
| XP_002934067.1 | 786 | TELVQWEKEDVAKYDHRLEVQFITDSTWVHSPNKYA-VVPWGLRKVLNWV | 834 |
| NP_004786.2 | 860 | KFKYGDLPMYIISNGIDDGLHAEDDQLRVYYMQNYINEALKAHILDGINL | 909 |
| XP_522655.2 | 860 | KFKYGDLPMYIISNGIDDGLHAEDDQLRVYYMQNYINEALKAQILDGINL | 909 |
| XP_001101127.1 | 862 | KFKYGDLPMYIISNGIDDGLHAEDDQLRVYYMQNYINEALKAHILDGINL | 911 |
| XP_003433357.1 | 712 | KFKYGDLPMYIVSNGIDDDPRAAQDSLRVYYMQNYVNEALKAYVLDGINL | 761 |
| NP_001178124.1 | 861 | KAKYGDLPMYIISNGIDDDPHAAQDNLRVYYMQTYVNEALKAYILDGINL | 910 |
| NP_038851.2 | 862 | RFKYGDLPMYVTANGIDDDPHAEQDSLRIYYIKNYVNEALKAYVLDDINL | 911 |
| NP_112626.1 | 862 | RFKYGDLPMFVTANGIDDDPHAEQDSLRMYYIKNYVNEALKAYVLDGINL | 911 |
| XP_004938814.1 | 873 | KSKYGDVPVYVMANGIDDDQNMVHDKLRVYYIQNYINEALKAYALDNVNL | 922 |
| XP_690797.4 | 837 | NSRYKGVPIYVMANGVQEDTARFRDSLRSYYLYNYVNEALKAYMLDAVNL | 886 |
| XP_002934067.1 | 835 | KSKYGDVPIYILANGIDDAHSPMQDKLRVYYLQNYINEALKAILHDGVNL | 884 |
| NP_004786.2 | 910 | CGYFAYSENDRTAPRFGLYRYAADQFEPKASMKHYRKIIDSNGFPGPETL | 959 |
| XP_522655.2 | 910 | CGYFAYSENDRTAPRFGLYRYAADQFEPKASMKHYRKIIDSNGFPGPETL | 959 |
| XP_001101127.1 | 912 | CGYFAYSFNDRTAPRFGLYRFAADQFEPKPSMKHYRKIIDSNGFPGPETL | 961 |
| XP_003433357.1 | 762 | CGYFAYSFNDRTAPKFGLYHYAANQFEPKPSVKHYRKIIDNNGFPGPETL | 811 |
| NP_001178124.1 | 911 | CGYFAYSFNDRTAPKFGLYRYAANQFEPKPSMKHYRKIIDNNGFPGPETL | 960 |
| NP_038851.2 | 912 | CGYFAYSLSDRSAPKSGFYRYAANQFEPKPSMKHYRKIIDSNGFLGSGTL | 961 |
| NP_112626.1 | 912 | CGYFAYSLSDRSVPKSGFYRYAANQFEPKPSIKHYRKIIDNNGFLGSGTL | 961 |
| XP_004938814.1 | 923 | QGYFVYSFNDKTAPKYGLYSYAANQYEPKPSMKHYREIIDNNGFPGPDTA | 972 |
| XP_690797.4 | 887 | KGYFAYAFSDQRDPGFGMYGHVQEEVISKSSLGHYKNIIRHNGFPAPSTS | 936 |
| XP_002934067.1 | 885 | RGYFAYSFNDRMDPRYGLYAYAANRFAPKLSMKHYQEIIDNNGFPNPEMP | 934 |
| NP_004786.2 | 960 | ERFCPEEFTVCTECSFFHTRKSLLAFIAFLFFASIISLSLIFYYSKKGRR | 1009 |
| XP_522655.2 | 960 | ERFCPEEFTVCTECSFFHTRKSLLAFIAFLFFAFIISLSLIFYYSKKGRR | 1009 |
| XP_001101127.1 | 962 | EKFCPEEFTVCTECSFFHTRKPLVAFIAFLFFAFIVSLSLIFYYSKKGRR | 1011 |
| XP_003433357.1 | 812 | GRFCPEEFTLCTECSFFHTRKSLLAFIAFLLFAFIISLSLIFYYSRKGRR | 861 |
| NP_001178124.1 | 961 | GRFCPEEFTLCTECSFFHTRKBLLAFIAFLLFAFVISLSLIFYYSKKGRR | 1010 |
| NP_038851.2 | 962 | GRFCPEEYTVCTECGFFQTRKSLLVFISFLVFTFIISLALIFHYSKKGQR | 1011 |
| NP_112626.1 | 962 | GRFCPEEYTVCTGCGFFQTRKSLLAFISFLVFAFVTSLALIYYYSKKGRR | 1011 |
| XP_004938814.1 | 973 | EVLCPEEAAMCPECHFFRTRKSLFAFISFIFVAFIVTIFCIMYYSKRAER | 1022 |
| XP_690797.4 | 937 | QHQCPHAPAQGSG-RYVLTKKPVVGFLSLVSSCMLITMCLVIYYAFKRHK | 985 |
| XP_002934067.1 | 985 | AVSCPVELVPCSDCHFFQTRKYLLAFVAFIFIVLIVSVEMITYYSRKGKR | 984 |
| NP_004786.2 | 1010 | --SYK | 1012 |
| XP_522655.2 | 1010 | --SYK | 1012 |
| XP_001101127.1 | 1012 | --RYK | 1014 |
| XP_003433357.1 | 862 | --SYK | 864 |
| NP_001178124.1 | 1011 | --GYK | 1013 |
| NP_038851.2 | 1012 | --SYK | 1014 |
| NP_112626.1 | 1012 | --RYK | 1014 |
| XP_004938814.1 | 1023 | --RYK | 1025 |
| XP_690797.4 | 986 | LTTKK | 990 |
| XP_002934067.1 | 985 | --RYK | 987 |

| Protein Acc. | Gene | Organism |
|---|---|---|
| NP_004786.2 | KL | H. sapiens |
| XP_522655.2 | KI | P. troglodytes |
| XP_001101127.1 | LOC714042 | M. mulatta |
| XP_003433357.1 | KL | C. lupus |
| NP_001178124.1 | KL | B. taurus |
| NP_038851.2 | Kl | M. musculus |
| NP_112626.1 | Kl | R. norvegicus |
| XP_004938814.1 | KL | G. gallus |
| XP_690797.4 | kl | D. rerio |
| XP_002934067.1 | kl | X. tropicalis |

The following is claimed:

1. A method of treating a disease associated with fibroblast growth factor 23 (FGF23) in a subject in need thereof comprising: administering a soluble α-klotho compound to the subject in an amount effective to treat the disease.

2. A method of preventing a disease associated with fibroblast growth factor 23 (FGF23) in a subject in need thereof comprising: administering a soluble α-klotho compound to the subject in an amount effective to prevent the disease.

3. A method of treating a disease associated with fibroblast growth factor 23 (FGF23) in a subject in need thereof comprising: increasing the expression of a soluble α-klotho compound in the subject to an extent effective to treat the disease.

4. A method of preventing a disease associated with fibroblast growth factor 23 (FGF23) in a subject in need thereof comprising: increasing the expression of a soluble α-klotho compound in the subject to an extent effective to prevent the disease.

5. A medicament for the treatment and/or prevention of a disease associated with fibroblast growth factor 23 (FGF23) comprising: a therapeutically effective amount of a soluble α-klotho compound.

6. A soluble α-klotho compound for use in the treatment or prevention of a disease associated with fibroblast growth factor 23 (FGF23).

7. A substance for treating or preventing a disease associated with fibroblast growth factor 23 (FGF23) comprising a soluble α-klotho compound as a main ingredient.

8. A use of a soluble α-klotho compound for the manufacture of a medicament for the treatment of a disease associated with fibroblast growth factor 23 (FGF23).

9. Any one of claims 1-8, wherein the disease associated with FGF23 is at least one of the following: kidney disease, chronic kidney disease, hyperphosphatemia, cardiovascular disease, vascular calcification, cardiovascular fibrosis, stress-induced cardiac injury, apoptosis, oxidative stress, cellular senescence, normal aging, tissue fibrosis, and inflammation.

10. A method of modulating the function of fibroblast growth factor 23 (FGF23) in a subject comprising administering a soluble α-klotho compound to the subject.

11. A method of reducing or eliminating hypertrophy in a cardiac myocyte associated with fibroblast growth factor 23 (FGF23), the method comprising exposing the cardiac myocyte to an effective amount of a soluble α-klotho compound.

12. A soluble α-klotho (sKL) compound comprising an amino acid sequence having less than 100% sequence identity with positions 34-981 of human klotho or positions 35-982 of mouse klotho.

13. Any one of claims 1-8 and 10-12, in which the soluble α-klotho compound comprises an amino acid sequence having at least 70% sequence identity with positions 34-981 of human klotho or positions 35-982 of mouse klotho.

14. Any one of claims 1-8 and 10-12, in which the soluble α-klotho compound comprises an amino acid sequence having at least 80% sequence identity with positions 34-981 of human klotho or positions 35-982 of mouse klotho.

15. Any one of claims 1-8 and 10-12, in which the soluble α-klotho compound comprises an amino acid sequence having at least 90% sequence identity with positions 34-981 of human klotho or positions 35-982 of mouse klotho.

16. Any one of claims 1-8 and 10-12, in which the soluble α-klotho compound comprises an amino acid sequence having at least 90% sequence identity with positions 34-981 of human klotho or positions 35-982 of mouse klotho.

17. Any one of claims 1-8 and 10-12, in which the soluble α-klotho compound comprises an amino acid sequence having at least 95% sequence identity with positions 34-981 of human klotho or positions 35-982 of mouse klotho.

18. Any one of claims 1-8 and 10-12, in which the soluble α-klotho compound has reduced glycosylation compared to wild type.

19. Any one of claims 1-8 and 10-12, in which the soluble α-klotho compound comprises a mutation that reduces the glycosylation of the soluble α-klotho compound compared to wild type.

20. Any one of claims 1-8 and 10-12, in which the soluble α-klotho compound comprises a mutation that increases the binding affinity of the soluble α-klotho compound to FGF23 compared to wild type.

21. Any one of claims 1-8 and 10-12, in which the soluble α-klotho compound comprises a mutation that increases stability of the soluble α-klotho compound to FGF23 compared to wild type.

22. Any one of claims 1-8 and 10-12, in which the soluble α-klotho compound comprises a mutation that is a substitution at one or more of positions 106, 159, 283, 344, 607, 612, 630, and 694.

23. Any one of claims 1-8 and 10-12, in which the soluble α-klotho compound comprises a mutation that is an N→Q substitution at one or more of positions 106, 159, 283, 344, 607, 612, 630, and 694.

24. Any one of claims 1-8 and 10-12, in which the soluble α-klotho compound comprises a substitution at a position corresponding to position 694 of human sKL.

25. Any one of claims 1-8 and 10-12, in which the soluble α-klotho compound comprises a mutation that is an N→Q substitution at a position corresponding to position 694 of human sKL.

26. The sKL compound of claim 12, in which the soluble α-klotho compound comprises a mutation that reduces the glycosylation of the soluble α-klotho compound compared to wild type.

27. The sKL compound of claim 12, in which the soluble α-klotho compound comprises a mutation that increases the binding affinity of the soluble α-klotho compound to FGF23 compared to wild type.

28. The sKL compound of claim 12, in which the soluble α-klotho compound comprises a mutation that increases stability of the soluble α-klotho compound to FGF23 compared to wild type.

29. The sKL compound of claim 12, in which the soluble α-klotho compound comprises a mutation that is a substitution at one or more of the following positions: 106, 159, 283, 344, 607, 612, 630, and 694.

30. The sKL compound of claim 12, in which the soluble α-klotho compound comprises one or more N→Q substitutions at one or more of the following positions: 106, 159, 283, 344, 607, 612, 630, and 694.

31. The sKL compound of claim 12, in which the soluble α-klotho compound comprises a substitution at a position corresponding to position 694 of human sKL.

32. The sKL compound of claim 12, in which the soluble α-klotho compound comprises a mutation that is an N→Q substitution at a position corresponding to position 694 of human sKL.

33. A nucleic acid that encodes the sKL compound of any one of claims 12 and 24-32.

34. A genetically modified cell comprising a nucleic acid that encodes the sKL compound of any one of claims 12 and 24-32.

35. A genetically modified animal comprising a nucleic acid that encodes the sKL compound of any one of claims 12 and 24-32.

36. The genetically modified animal comprising a nucleic acid that encodes the sKL compound of any one of claims 12 and 24-32, wherein the animal is a non-human animal.

37. A method of making an sKL compound, comprising expressing a nucleic acid that encodes the sKL compound of any one of claims 12 and 24-32.


SEQUENCE LISTING (10 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - protein, 1012 aa, Pan troglodytes
MPASAPPRRPRPPPPSLSLLLVLLALGGRRLRAEPGDGAQTWARFSRPPAPEAAGLFQGTFPDGFLWAVGSAAYQTEGGWQQHGKGASIWDTFTHHPLAPPGDSRNASLPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTEHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHITHFRFSLDWALILPLGNQSQVNHTILQYYRCMASELVRVNITPVVALWQPMAPNQGLPRLLARQGAWENPYTALAFAEYARLCFQALGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAQILDGINLCGYFAYSFNDRTAPRFGLYRYAADQFEPKASMKHYRKIIDSNGFPGPETLERFCPEEFTVCTECSFFHTRKSLLAFIAFLFFAFIISLSLIFYYSKKGRRSYK

SEQ ID NO: 3 - protein, 1014 aa, Macaca mulatta
MPASAPPRRLRPPPPSLSLSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 4 - protein, 864 aa, Canis lupus
MATCILQMRFLRLGKILFHSSPQSTGGSGGTRGPRAPAQLRTQRGTDKLVAKSELKAKTAHRALADHFRDYAELCFRHFCGQVKYWITIDNPYVVAWHGYATGRLAPGVRGSPRLGYLVAHNLLLAHAKIWHLYNTSFRPTQGGQVSIALSSHWINPRRMTDHSIKECQKSLDFVLGWFAKPIFIDGDYPESMKNNLSSLLPVFTESEKKFIKGTADFFALSFGPTLSFQLLDPHMKFHQLESPSLRQLLSWIDLEYNHPQIFIVENGWFVSGTTKRDDAKYMYYLKKFIMETLKAIRLDGVDVIGYTAWSLMDGFEWHRGYSIRRGLFYVDFLSQDKKLLPKSSALFYQKLIEKNGFPPLPENQPLEGTFPCDFAWGIVDNYIQVDTTLSQFTDPNVYLWDVHHSKRLIKVDGLRAKKRKPYCVDFAAIGPQVALLQEMHVSHFHFSLDWALLLPLGNQSRVNHAALHYYGCVASELLRANITPVVALWRPAAAHQGLPGPLAQRGAWENPRTALAFAEYARLCFRALGRHVKVWITLREPPTRNLTLRAGHNLLRAHALAWRVYDEQFRGSQQGKVSIALQADWVEPACPSSQKDREVAERVLEFDVGWLAEPIFGSGDYPRLMRDWLTRRDHSLLPYFTDEEKRLIRGSFDFLALSHYTTILVDWEKEDPVKYNDYLEVQEMTDITWLNSPSQVAVVPWGLRKVLNWLKFKYGDLPMYIVSNGIDDDPRAAQDSLRVYYMQNYVNEALKAYVLDGINLCGYFAYSFNDRTAPKFGLYHYAANQFEPKPSVKHYRKIIDNNGFPGPETLGRFCPEEFTLCTECSFFHTRKSLLAFIAFLLFAFIISLSLIFYYSRKGRRSYK

SEQ ID NO: 5 - protein, 1013 aa, Bos taurus
MPARAPPRRLRAFPPPPPPLWLLLLALGGRPLRAEPGDGAQTWARFARPPTPEAAGLLHDTFPDGFLWAVGSAAYQTEGGWQQHGKGASIWDTFTHRPPAPPGDPSAAGWPSGAPSPPPPATGDVASDGYNNVFRDTEGLRELGVTHYRFSISWARVLPNGSASAPNREGLRYYRRLLERLRELGVQPVVTLYHWDLPQRLQDAYGGWANRALADHFRDYAELCFRHFGGQVKYWITIDNPYVVAWHGYATGRLAPGVRGSPRLGYLVAHNLLLAHAKIWHLYDTSFRPTQGGQVSIALSSHWISPRRMTEHSIQECQKSLDFVLGWFAKPIFIDGDYPESMKNNLSSLLPDFTESEKKFIKGTADFFALSFGPTLSFQLLDPQMKFRQLESPSLRQLLSWIDLEYNHPQIFIVENGWFVSGTTKRDDAKYMYYLKKFIMETLKAIRLDGVDVIGYTAWSLMDGFEWHRGYSIRRGLFYVDFLSQDKKLLPKSSALFYQKLIENNGFPPLPENQPLEGTFPCDFAWGVVDNCIQVDTTLSQFIDPNVYLWDVHRSKRLIKVDGVLTKTRKSYCVDFAAIRPQIALLQEMHVTHFHFSLDWALILPLGNRSQVNRTVLGFYRCVASELVRANITPVVALWRPAAPHQGLPAPLARHGAWENPHTALAFAEYASLCFQDLGRHVKFWITMHEPSTRNMTYSAGHNLLKAHALAWRTYDERFRRSQKGKISIALQADWIEPACPFSPEDREVAERVLEFDIGWLAEPIFGSGDYPRVMRDWLNQRNNFLLPYFTDEEKKLIRGSFDFLALSHYTTILVDWEKEDPIKYNDYLAVQEMTDITWLNSPSQVAVVPWGLRKVLNWLKAKYGDLPMYIISNGIDDDPHAAQDNLRVYYMQTYVNEALKAYILDGINLCGYFAYSFNDRTAPKFGLYRYAANQFEPKPSMKHYRKIIDNNGFPGPETLGRFCPEEFTLCTECSFFHTRKSLLAFIAFLLFAFVISLSLIFYYSKKGRRGYK

SEQ ID NO: 6 - protein, 1014 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVDTTLSQFTDPNVYLWDVHHSKRLIKVDGVVAKKRKPYCVDFSAIRPQITLLREMRVTHFRFSLDWALILPLGNQTQVNHTVLHFYRCMISELVHANITPVVALWQPAAPHQGLPHALAKHGAWENPHTALAFADYANLCFKELGHWVNLWITMNEPNTRNMTYRAGHHLLRAHALAWHLYDDKFRAAQKGKISIALQADWIEPACPFSQNDKEVAERVLEFDIGWLAEPIFGSGDYPRVMRDWLNQKNNFLLPYFTEDEKKLVRGSFDFLAVSHYTTILVDWEKEDPMKYNDYLEVQEMTDITWLNSPSQVAVVPWGLRKVLNWLRFKYGDLPMYVTANGIDDDPHAEQDSLRIYYIKNYVNEALKAYVLDDINLCGYFAYSLSDRSAPKSGFYRYAANQFEPKPSMKHYRKIIDSNGFLGSGTLGRFCPEEYTVCTECGFFQTRKSLLVFISFLVFTFIISLALIFHYSKKGQRSYK

SEQ ID NO: 7 - protein, 1014 aa, Rattus norvegicus
MPARAPPRRLPRLLLLRLLSLHLLLLTLRARCLSAEPGQGAQTWARFARPPVPEASGLLHDTFPDGFLWAVGSAAYQTEGGWRQHGKGASIWDTFTHHPRAIPEDSPIVMAPSGAPLPPLPSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWRLYNTSFRPTQGGRVSIALGSHWITPRRMTDYHIRECQKSLDFVLGWFAKPIFIDGDYPKSMKNNLSSLLPDFTESEKRFIRGTADFFALSFGPTLSFQLLDPSMKFRQLESPSLRQLLSWIDLEYNHPQIFIVENGWFVSGTTRRDDAKYMYYLKKFIMESLKAIRLDGVDVIGYTAWSLMDGFEWHRGYSIRRGLFYVDFLSQDKELLPKSSALFYQKLIENNGFPPLPENQPLEGTFPCDFAWGVVDNYIQVDPTLSQFTDPNVYLWDVHHSKRLIKVDGVVAKKRKPYCVDFSAIRPQITLLREMRVTHFRFSLDWALILPLGNQTQVNRTVLHFYRCMVSELVHANITPVVALWQPATPHQGLPHALAKHGAWENPHTALAFADYANLCFEELGHWVKFWITINEPNSRNMTYRAGHHLLKAHALAWHLYDDKFRAAQKGKISIALQVDWIEPACPFSQKDKEVAERVLEFDVGWLAEPIFGSGDYPHVMREWLNQKNNFLLPYFTEDEKKLIRGSFDFLALSHYTTILVDWEKEDPIKYNDYLEVQEMTDITWLNSPNQVAVVPWGLRKALNWLRFKYGDLPMFVTANGIDDDPHAEQDSLRMYYIKNYVNEALKAYVLDGINLCGYFAYSLSDRSVPKSGFYRYAANQFEPKPSIKHYRKIIDNNGFLGSGTLGRFCPEEYTVCTGCGFFQTRKSLLAFISFLVFAFVTSLALIYYYSKKGRRRYK

SEQ ID NO: 8 - protein, 1025 aa, Gallus gallus
MAPPPPPVLPVLPVLLVLLGLGRPLLGAPGQGAGTWARFAHLPYPQDQLFLHDTFPDGFLWGAGSAAYQTEGGWRQGGKGASIWDTFTHRPTTPAGSILPGPTGGDVASDSYNNIFRDIEGLRHLGVSHYRFSLAWTRLMPNGTAPVNPVGLAHYGQVLSRLRELGIEPIVTLYHWDLPQGLQDAFGGWASPVLPNLFHDYAELCFRHFGGQVRYWLTMDNPYVVAWHGYGTGRLPPGVQGGPSLGYRAAHHLLQSRCTAVGALFGTLCIVTLKAVGLHTRAHAKVWHLYNDHFRPTQKGKVSIALSSHWIKPQHMTEKNIKECQKSLDFVLGWFAKPIFINGDYPESMRSNLSSLLPEFSEEDKKYIKGTADFFALSFGATLSFQLLDSHMKFQQLESISLRQLLYWISTEYNNPPVFIVENSWFVSGSTKRDDAKYIYYLKKFIMETLKAIRYDGVNVFGYTVWSLLDGFEWHRGYSIRRGLFYVDFQSHDKKLIPKSSVLFYQKLIEKNGFPPLPENQPIAGVFPCGFAWGIVDNYIQVDTTPAQFLDPNVYVWDVHQTKKLIKVDGVFTSQRKHHCVDFAAIRLQISLLQEMHVTHFHFSLKWSSVLPLGNLSLINHTLVHYYQCFASELLRVNITPVVALWQPMAENQELPTSLAKFGAWENSETVQAFVEYAKFCFASLGDHVKFWITMNEPSVKNLTYTAGHNLLRAHAKAWHLYDKEFRRSQKGKISIALQADWVEPACPFSRNDQEVADRILEFDIGWLAEPIFGSGDYPHMMRAWLHQRNSVDLYNFHLPSFSEDEKKLIQGSFDFFALSHYTTILVGWEKEDALKYDHYLEVQMISDITWLHSPSRAAVVPWGLRKVLRWVKSKYGDVPVYVMANGIDDDQNMVHDKLRVYYIQNYINEALKAYALDNVNLQGYFVYSFNDKTAPKYGLYSYAANQYEPKPSMKHYREIIDNNGFPGPDTAEVLCPEEAAMCPECHFFRTRKSLFAFISFIFVAFIVTIFCIMYYSKRAERRYK

SEQ ID NO: 9 - protein, 990 aa, Danio rerio
MKVTWIPLPLVLFCQFQGTASDPGAGQHTWDTFSKLPYPDDKAFLYDTFPDKFMWAVGTAAYSVEGAWEKDGKGKSIWDTFTRGGTRVSRGDVGSDSYHNIPGDLRALQQLGVSHYRFSLSWPRIFSNGTKESYNDKGVEYYKNLIRGLKDIKVQPVVTLYHWDLPDSLQTLFGGWSNSVMVELFRDYADFCFKTFGSDVKFWITIDNPFVVAWHGYGTGVVAPGIKNDSDLPFRVGHNLLKAHAAAWHLYDERYRAAQGGRVSMALGSHWIKPSRTRQESRKACQRSLNFVLGWFARPLFVDGDYPPCMKDNLTHRLPSFTEAESAYVNGTADFFALSHGPALSFQLINDSLRFGQTEDLGLRMLLYWVRAEYNNPPIFVVESGWYGSGNTKTKDAKHMYYLKRFIMETLKAIHVDRVNVIGYTAWSLLDGYEWYREYAIRRGLFYVDFNTPDLKREPKASATFYSKLIEKNGFPQLPENRPAQGAFPCDFAWGVAANSIQVDTTPTQFTDTNVYVWNISGNGELKKLPGLQAPHLRRTPHCADYGSIRQQVSDLLRMQVSHFHFSLNWSSIVPTGHVSDANETLLRYYYCFVSELQKVNITPVVTLWHHTGKLSSLPAPMEASDGWQSEKTVQAFVDYARLCFQRLGAHVKLWITLNEPNDEDLEYTVGHQLLRAHALAWHVYDREFRKAQGGKASLVLHMDWVEPAFSFNREDVAPADRVLDFRVGWFAEPIFGKGDYPAVMRSWLQQRNTIDLFNYHLPTFSEEDRLLVKGTYDFFAISHFTTSMVYDGVEDKYTFKDKLQVQLISDVTWIMSPRRNSPVVPWGLRKALNWVNSRYKGVPIYVMANGVQEDTARFRDSLRSYYLYNYVNEALKAYMLDAVNLKGYFAYAFSDQRDPGFGMYGHVQEEVISKSSLGHYKNIIRHNGFPAPSTSQHQCPHAPAQGSGRYVLTKKPVVGFLSLVSSCMLITMCLVIYYAFKRHKLTTKK

SEQ ID NO: 10 - protein, 987 aa, Xenopus tropicalis
MRPGALALLWAAAWGCALCALCGNAEKNVWSRFAQLPFPQDNLFLYGTFPPNFMWSVGTAAYQVEGGWEQNGKAPSIWDTFCHKSGQLDATGDVASDSYNNLFRDTEALKSLGVTHYRFSISWPRLFPNGTESAPNEAGLSYYRNLILRLKELRIEPVVTLYHWDLPQRLQDVYGGWVSESMVGIFRDYAEACFRLLGDEVKYWITIDNPYVVAWQGYATGKVPPGIKGDKVMGYRAGHNIIKAHAAVWHLYDKKFRPQQGGQISIALASHWINPVNMTSHDIGDCQKSLDFALGWFAKPIFIDGDYPQTMKNNLSSLLPEFSDQEKKLNKGTADFFSLSFGPNLSFQMLDPDMKFRQVESTSLRKILYWINEQYNKPRIFIVENSWFLSGNTKREDAKYMYYLKKFVMETLKAIKYDGVKVIGYTAWSLMDGFEWLREYTIRRGLFYVDFTSHNKKLMPKSSALFYQQLIKMNGFPPLPENQPVEGTFPCDFAWGTSAYRIHIDTTPSQFNDPNVYVWDMNKTKGLTKVEGITVPKRKTQCVDFASIRQQISMLREIHITHFYFALKWAAILPLGNLSLIHHKVLHYYQCFVSELVRVNITPVVALWQPLAENQGLPIDLAKNGGWVNYHTVSAFVEYARLCFKELGNYVGMWITMNEPSMRNLTYAAGHNLLKAHALAWHLYDRDFRKAQKGQISIAVQADWVEPASPFSKNDKETSRRILEFEIGRLSDPIFLSGDYPKVMRDWLAPRNNLDVFEFYLPSFTEEEKNLIQGTFDFFALSHFTTELVQWEKEDVAKYDHRLEVQFITDSTWVHSPNKYAVVPWGLRKVLNWVKSKYGDVPIYILANGIDDAHSPMQDKLRVYYLQNYINEALKAILHDGVNLRGYFAYSFNDRMDPRYGLYAYAANRFAPKLSMKHYQEIIDNNGFPNPEMPAVSCPVELVPCSDCHFFQTRKYLLAFVAFIFIVLIVSVFMITYYSRKGKRRYK